(19) United States
(12) Patent Application Publication
(10) Pub. No.: US 2009/0066898 A1
(43) Pub. Date: Mar. 12, 2009

(54) OPTICALLY-COMPENSATORY SHEET, ELLIPSOIDAL POLARIZING PLATE AND LIQUID CRYSTAL DISPLAY DEVICE

(75) Inventors: Tomonori Ichinose, Kanagawa (JP); Yohei Takahashi, Kanagawa (JP); Tokuju Oikawa, Kanagawa (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/281,674

(22) PCT Filed: Mar. 13, 2007
(86) PCT No.: PCT/JP2007/055587

§ 371 Date: Sep. 4, 2008

(30) Foreign Application Priority Data
Mar. 13, 2006 (JP) 2006-067587
Mar. 22, 2006 (JP) 2006-078519

(51) Int. Cl. G02F 1/1335; C09K 19/54; C07C 43/21

(52) U.S. Cl. 349/117; 252/299.5; 568/609

(57) ABSTRACT

An optically-compensatory sheet comprises an optically anisotropic layer that comprises a liquid crystal compound, wherein the liquid crystal compound in the optically anisotropic layer has Sₘₐₓ of 0.4 or more, in which Sₘₐₓ is a maximum value among S₁, S₂ and S₃, S₁ is an alignment order parameter in a vicinity of an interface between the optically anisotropic layer and air; S₂ is an alignment order parameter in a vicinity of an interface on a side opposite to the interface between the optically anisotropic layer and air; and S₃ is an alignment order parameter in an interior of the optically anisotropic layer.

### TECHNICAL FIELD

The present invention relates to an optically-compensatory sheet comprising an optically anisotropic layer having liquid crystal molecules fixed aligned therein, an ellipsoidal polarizing plate and a liquid crystal display device comprising the optically-compensatory sheet.

### BACKGROUND ART

An optically-compensatory sheet is used to extinguish image color or enhance the viewing angle in various liquid crystal display devices, As such an optically-compensatory sheet there has heretofore been used a stretched birefringent film. In recent years, it has been proposed that an optically-compensatory sheet having an optically anisotropic layer formed on a transparent support by a discotic liquid crystal compound be used instead of the stretched birefringent film. This optically anisotropic layer is normally formed by spreading a discotic liquid crystal composition containing a discotic liquid crystal compound over an alignment film, heating the coated material at a temperature higher than the alignment temperature so that the discotic liquid crystal compound molecules are aligned, and then fixing the discotic liquid crystal compound molecules aligned. In general, a discotic liquid crystal compound has a great birefringence and a variety of alignment configurations. The use of such a discotic liquid crystal compound made it possible to realize optical properties that would never be obtained by the related art stretched birefringent films.

On the other hand, since a discotic liquid crystal compound has a variety of alignment configurations, it is necessary that the alignment of the discotic liquid crystal compound in the optically anisotropic layer be properly controlled to develop the desired optical properties. As a method for controlling the discotic liquid crystal compound aligned horizontally at an average tilt angle of less than 5° there is proposed a method which comprises adding a cellulose lower aliphatic acid ester, a fluorine-containing surface active agent or a compound having 1,3,5-triazine ring to a discotic liquid crystal compound (see, e.g., JP-A-11-352328). Further, a method is proposed which comprises adding a compound having a fluorine-substituted alkyl group and a hydroxyl group (having a sulfo group connected to benzene ring via linkage) to an optically anisotropic layer to control the tilt angle of the discotic liquid crystal compound (see, e.g., JP-A-2001-330725). Moreover, a method is proposed which comprises using a hydrophobicity-excluding volume effect compound and a liquid crystal compound in combination to control the alignment of the liquid crystal compound (see, e.g., JP-A-2002-20363). However, these patent references have no reference to the effect and use of a compound which accelerates effectively the hybrid alignment of liquid crystal compound. Therefore, in order to obtain desired hybrid alignment or optical compensation effect, it is necessary that the thickness of the optically anisotropic layer be raised. As a result, defects can easily occur at the step of curing the optically anisotropic layer, causing the rise of haze or requiring the use of a large amount of coating solution for the formation of the optically anisotropic layer. Therefore, these methods are disadvantageous in that the productivity is lowered.

### DISCLOSURE OF THE INVENTION

A object of the invention is to provide an optically-compensatory sheet which exhibits an excellent optical compensation effect to contribute to the enhancement of the viewing angle properties of liquid crystal display devices, particularly an optically-compensatory sheet which can exhibit the same optical compensation effect as the related art optically-compensatory sheet even when it is thinner than the related art optically-compensatory sheet, can be produced at a high productivity and exhibits a good haze. Another object of the invention is to provide an ellipsoidal polarizing plate which contributes to the enhancement of the viewing angle of liquid crystal display devices and a liquid crystal display device having a high display fidelity.

The aforementioned objects can be achieved by the following constitutions.

[1] An optically-compensatory sheet comprising:

an optically anisotropic layer that comprises a liquid crystal compound,

wherein the liquid crystal compound in the optically anisotropic layer has Sₘₐₓ of 0.4 or more,

in which Sₘₐₓ is a maximum value among S₁, S₂ and S₃,

S₁ is an alignment order parameter in a vicinity of an interface between the optically anisotropic layer and air,

S₂ is an alignment order parameter in a vicinity of an interface on a side opposite to the interface between the optically anisotropic layer and air; and

S₃ is an alignment order parameter in an interior of the optically anisotropic layer.

[2] The optically-compensatory sheet as described in above [1],

wherein S₃ is Sₘₐₓ.

[3] The optically-compensatory sheet as described in above [1],

wherein S₁ is substantially same as Sₘₐₓ.

[4] The optically-compensatory sheet as described in above [1],

wherein S₂ is substantially same as Sₘₐₓ.

[5] The optically-compensatory sheet as described in above [1],

wherein S₁, S₂ and S₃ are substantially same.

[6] The optically-compensatory sheet as described in above [1],

wherein θ1 is greater than θ2,

in which θ1 is an angle of inclination of the liquid crystal compound in the vicinity of the interface between the optically anisotropic layer and air; and

θ2 is an angle of inclination of the liquid crystal compound in the vicinity of the interface on the side opposite to the interface between the optically anisotropic layer and air.

[7] The optically-compensatory sheet as described in above [6],

wherein θ1 is 40 degrees or more.

[8] The optically-compensatory sheet as described in above [1],

wherein Sₘₐₓ is 0.5 or more.

[9] An optically-compensatory sheet comprising:

an optically anisotropic layer that comprises a liquid crystal compound,

wherein the liquid crystal compound in the optically anisotropic layer has θ1 of 55 degrees or more and Si of from 0.35 to 0.7,

in which θ1 is an angle of inclination of the liquid crystal compound in a vicinity of an interface between the optically anisotropic layer and air; and

S₁ is an alignment order parameter of the liquid crystal compound in the vicinity of the interface between the optically anisotropic layer and air

[10] The optically-compensatory sheet as described in above [1] or [9],

wherein the optically anisotropic layer further comprises at least two fluoroaliphatic group-containing polymers, each of which comprises a repeating unit derived from a monomer represented by formula (1):

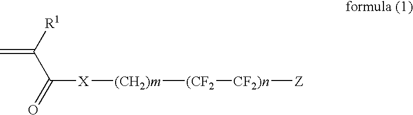

wherein R¹ represents a hydrogen atom or methyl group;

X represents an oxygen atom, sulfur atom or N(R²)— in which R² represents a hydrogen atom or C₁-C₄ alkyl group;

Z represents a hydrogen atom or fluorine atom;

m represents an integer of from 1 to 6; and

n represents an integer of from 2 to 4

[11] The optically-compensatory sheet as described in above [10], wherein Z represents a hydrogen atom.

[12] The optically-compensatory sheet as described in above [10],

wherein at least one fluoroaliphatic group-containing polymer of the at least two fluoroaliphatic group-containing polymers further comprises a repeating unit derived from a monomer represented by formula (2):

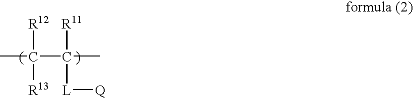

wherein R¹¹, R¹² and R¹³ each independently represents a hydrogen atom or a substituent;

L represents a divalent connecting group which is a combination of one or more connecting groups selected from a group consisting of single bond; —O—; —CO—; —NR⁴— in which R⁴ represents a hydrogen atom, alkyl group, aryl group or aralkyl group; —S—; —SO₂—; —P(═O)(OR⁵)— in which R⁵ represents an alkyl group, aryl group or aralkyl group; alkylene group and arylene group; and

Q represents a carboxyl group (—COOH), salt thereof, sulfo group (—SO₃H), salt thereof, phosphonoxy {—OP(═O)(OH)₂}, salt thereof or alkylene group containing poly(alkyleneoxy) group.

[13] The optically-compensatory sheet as described in above [1] or [9],

wherein the liquid crystal compound is a discotic compound.

[14] An ellipsoidal polarizing plate comprising:

a polarizing film; and

an optically-compensatory sheet as described in above [1] or [9].

[15] A liquid crystal display device comprising an optically-compensatory sheet as described in above [1] or [9].

### BEST MODE FOR CARRYING OUT THE INVENTION

The invention will be further described hereinafter. In the present specification, the expression “from A to B” is used to mean that the numerals A and B are included as the lower limit and the upper limit. Further, the term “on the support (or alignment film)” as used herein is meant to include both the case where something is provided directly on the surface of the support (or alignment film) and the case where something is provided on the support (or alignment film) with some layer (film) provided interposed therebetween.

In the invention, Re (λ) and Rth (λ) represent the in-plane retardation and thickness-direction retardation at a wavelength of λ, respectively. Re (λ) can be measured by the incidence of light having a wavelength λ nm in the direction normal to the film using a Type KOBRA 21ADH birefringence meter (produced by Ouji Scientific Instruments Co. Ltd.). Rth (λ) can be calculated by an automatic birefringence meter on the basis of retardation values measured in the total three directions, i.e., Re(λ), retardation value measured at 11 points by the incidence of light having a wavelength λ nm in the direction inclined at an angle of from −50° to +50° every 10° from the direction normal to the film with the in-plane slow axis (judged from “KOBRA 21ADH”) as an inclined axis (rotary axis), the hypothetical average refractive index and the film thickness inputted. As the hypothetical average refractive index there may be used one disclosed in “Polymer Handbook”, John Wiley & Sons, Inc. and various catalogues of optical films. For the cellulose acylate films having an unknown average refractive index, an Abbe refractometer may be used. The average refractive index of main optical films are exemplified below: Cellulose acylate (1.48), cycloolefin polymer (1.52), polycarbonate (1.59), polymethylene methacrylate (1.49), polystyrene (1.59). By inputting the hypothetic average refractive indexes and film thicknesses, KOBRA 21ADH calculates nₓ, n_{y} and n_{z}.

The optically-compensatory sheet of the invention comprises at least one optically anisotropic layer having liquid crystal molecules fixed aligned therein, wherein the requirement is satisfied that the maximum value Sₘₐₓ of the alignment order parameter S of the molecules in the optically anisotropic layer be not smaller than 0.4.

### (Alignment Order Parameter)

The alignment order parameter (hereinafter occasionally referred to as “S”) is used as an indication showing the alignment degree of polymer film or liquid crystal and is defined to fall within the range 0≦S≦1. When S is 0, it indicates a fully random state such as liquid, When S is 1, it indicates a fully unidirectionlly aligned state free of molecular fluctuation such as crystal. In general, the alignment order parameter of the crystalline polymer film is measured by X-ray diffraction pattern. However, for the measurement of a nematic liquid crystal film, this method gives a low sensitivity and thus is undesirable as a measuring method The term “alignment order parameter” as used herein is meant to indicate a value obtained by measuring a sample using “Nanofinder” (produced by TOKYO INSTRUMENTS, INC.) at an excitation laser wavelength of 532 nm with an excitation laser output of about 400 μW at the sample portion with a depolarizer mounted before the spectroscope. As the sample to be measured there is used one obtained by cutting the film to be measured obliquely at an angle of about 1° to 2° with respect to the surface of the film.

The aforementioned sample is irradiated with laser polarization to measure the polarized Raman of the optically anisotropic layer in the vicinity of air interface. The measurement is effected while the sample is being rotated such that the angle of the orientation of the surface of the sample on which laser polarization is incident with the direction of electric field of incident laser polarization is varied to give various measurement angles. The polarization component (I-parallel) parallel to the electric field of incident laser polarization and the polarization component (I-perpendicular) perpendicular to the electric field of incident laser polarization among the components of scattered light are spectrally detected using a detector. The band having a peak derived from the skeleton of molecules contained in the layer is subjected to fitting analysis based on minimum square method with alignment order parameters P2, P4 as a parameter according to a theoretically derived formula to obtain an alignment order parameter S.

In the invention, the maximum value Sax of alignment order parameter of molecules in the aforementioned optically anisotropic layer is preferably from not smaller than 0.4 to not greater than 1.0. However, since the optically anisotropic layer is not in crystalline state, the upper limit of the maximum vale Sₘₐₓ is normally from 0.7 to 0.8. The maximum value Sₘₐₓ of alignment order parameter is preferably not smaller than 0.5, more preferably not smaller than 0.6. Further, when the alignment order parameter falls within the above defined range, the thickness of the optically anisotropic layer can be reduced from that of the related art optically-compensatory sheet having the same display properties, making it possible to eliminate the occurrence of defects caused by the rise of the film thickness and hence provide an optically-compensatory sheet at an enhanced productivity.

The alignment order parameter S is substantially the same when it falls within the range of ±0.05 from measurement precision.

When the alignment order parameter is measured by the aforementioned measurement method, alignment order parameter is obtained for each of depth direction divisions, i.e., area in the vicinity of air interface, interior of the optically anisotropic layer and area in the vicinity of interface on the other side (e.g., alignment film interface). The alignment order parameter S obtained in area in the vicinity of air interface is occasionally referred to as “S₁”, the alignment order parameter S obtained in area in the vicinity of interface on the other side (e.g., alignment film interface) is occasionally referred to as “S₂” and the alignment order parameter S obtained in interior of the optically anisotropic layer is occasionally referred to as “S₃.” The alignment order parameter S obtained for these divisions may be the same or different. In the optically-compensatory sheet of the invention, the alignment order parameter measured for interior of the aforementioned optically anisotropic layer preferably shows a maximum value Sₘₐₓ. Further, in the case where the aforementioned optically anisotropic layer has an air interface, it is preferred that the alignment order parameter of area in the vicinity of the air interface be substantially the same as Sₘₐₓ and the alignment order parameter of area in the vicinity of the interface on the side of the aforementioned optically anisotropic layer opposite the air interface be substantially the same as Sₘₐₓ. It is more desirable that the alignment order parameter S measured for all these divisions be the same and the alignment order parameter be substantially constant all over the entire optically anisotropic layer. It is even more desirable that the alignment order parameter measured for all these divisions be 0.4 or more, particularly preferably 0.5 or more.

The alignment order parameter of the area in the vicinity of the air interface of the optically anisotropic layer and the area in the vicinity of the interface on the side of the optically anisotropic layer opposite the air interface each indicates the average of alignment order parameter measured from the respective interface to a depth of from 200 nm to 300 nm. The interior of the optically anisotropic layer is the area except the area in the vicinity of the aforementioned air interface and the area in the vicinity of the interface on the side opposite the air interface. In some detail, the interior of the optically anisotropic layer is the inner area except the area extending from the two interfaces of the optically anisotropic layer to a depth of from 200 nm to 300 nm.

The alignment state of the molecules in the aforementioned optically anisotropic layer is not specifically limited so far as the aforementioned requirements are satisfied. An embodiment of implementation of the invention concerns an optically-compensatory sheet wherein the aforementioned optically anisotropic layer has at least an air interface and the tilt angle θ1 of molecules in the vicinity of the air interface is greater than the tilt angle θ2 of molecules in the vicinity of the interface on the side opposite the air interface. The preferable average tilt angle of molecules in the vicinity of the air interface depends on the thickness of the optically anisotropic layer, and generally 40° or more is preferable, 60° or more is more preferable and even 80° or more is even more preferable.

The alignment order parameter of the optically anisotropic layer in the optically-compensatory sheet of the invention in the vicinity of the air interface is from not smaller than 0.35 to not greater than 0.7, preferably from not smaller than 0.4 to not greater than 0.65. The optically anisotropic layer having an alignment order parameter falling within the above defined range wherein the liquid crystal compound exhibits a tilt angle of from 55° to 75° at the air interface can be reduced in its thickness from that of the related art optically-compensatory sheets having the same display properties, making it possible to provide an optically-compensatory sheet which can be produced at a higher productivity and exhibits a good haze. In some detail, the thickness of the related art optically-compensatory sheets, which ranges from 1.5 μm to 1.6 μm, can be reduced to a range of from 1.25 μm to 1.35 μm in the invention, Further, the haze of the related art optically-compensatory sheets, which ranges from 0.5% to 1.2%, can be reduced to a range of from 0.3% to 0.4% in the invention.

Various materials to be used in the production of the optically-compensatory sheet of the invention will be described hereinafter.

### (Optically Anisotropic Layer)

The aforementioned optically anisotropic layer contained in the optically-compensatory sheet of the invention can be formed by a liquid crystal composition containing at least one liquid crystal compound. In particular, a liquid crystal composition containing at least one liquid crystal compound and at least two fluoroaliphatic group-containing polymers is preferably used.

When a fluoroaliphatic group-containing polymer which effectively accelerates the hybrid alignment of liquid crystal compounds is used, the amount of related art hybrid alignment accelerators causing the deterioration of alignment order parameter or haze can be reduced, making it possible to attain the aforementioned effect of the invention.

### (Fluoroaliphatic Group-Containing Polymer)

The fluoroaliphatic group-containing polymer (hereinafter occasionally abbreviated as “fluorine-based polymer”) which can be used to form the aforementioned optically anisotropic layer is preferably a polymer containing a repeating unit derived from the fluoroaliphatic group-containing monomer represented by formula (1) shown later. The aforementioned fluorine-based polymer is more preferably a copolymer containing a repeating unit derived from the monomer represented by formula (1) shown later. As the aforementioned fluorine-based polymer there is preferably used an acrylic resin or methacrylic resin having a fluoroaliphatic group. Further, an acrylic resin or methacrylic resin obtained by the copolymerization of the monomer represented by formula (1) with a vinyl-based monomer copolymerizable therewith is preferably used.

One of the fluoroaliphatic groups in the aforementioned fluorine-based polymer is one derived from fluoroaliphatic compounds produced by telomerization method (referred to also as “telomer method”) or oligomerization method (referred to also as “oligomer method”). For the details of method for the production of these fluoroaliphatic group compounds, reference can be made to “Fusso Kagobutsu no Gosei to Kinou (Synthesis and Function of Fluorine Compounds)”, compiled by Nobuo Ishigawa, issued by CMC, 1987, pp. 117-118, and Milos Hudlicky and Attila E. Pavlath, eds., “Chemistry of Organic Fluorine Compounds II”, Monograph 187, American Chemical Society, 1995, pp. 747-752. Telomerization method is a method which comprises subjecting a fluorine-containing vinyl compound such as tetrafluoroethylene to radical polymerization with an alkyl halide having a great chain transfer constant (e.g., iodide) as a telogen to synthesize a telomer (This method is exemplified in Scheme-1).

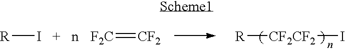

The iodine-terminated telomer thus obtained is normally chemically modified at the terminal thereof as shown in [Scheme-2] to form fluoroaliphatic compounds. These compounds are then optionally converted to desired monomer structures which are then used to produce fluoroaliphatic group-containing polymers.

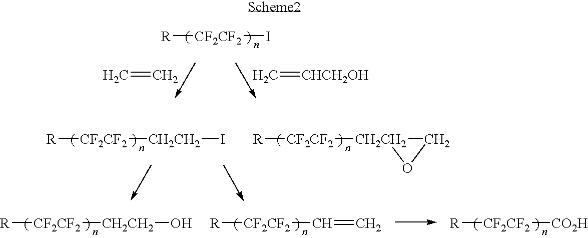

The aforementioned fluorine-based polymer contains repeating units derived from the fluoroaliphatic group-containing monomer represented by formula (1).

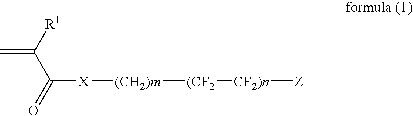

In formula (1), R¹ represents a hydrogen atom or methyl group; X represents an oxygen atom, sulfur atom or —N(R²)— in which R² represents a hydrogen atom or C₁-C₄ alkyl group, preferably a hydrogen atom or methyl group, Z represents a hydrogen atom or fluorine atom; m represents an integer of from not smaller than 1 to not greater than 6; and n represents an integer of from 2 to 4.

X is preferably an oxygen atom, Z is preferably a hydrogen atom, m is preferably 1 or 2, and n is preferably 3 or 4. These different repeating units may be used in admixture.

Specific examples of the monomer which can be used in the production of the fluorine-based polymer employable herein will be given, but the invention is not limited thereto.

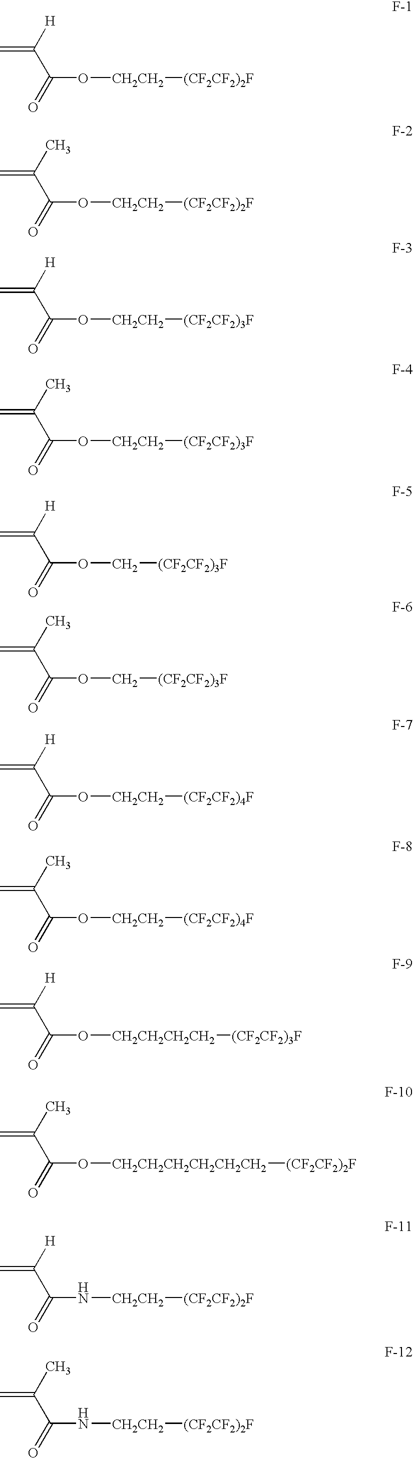

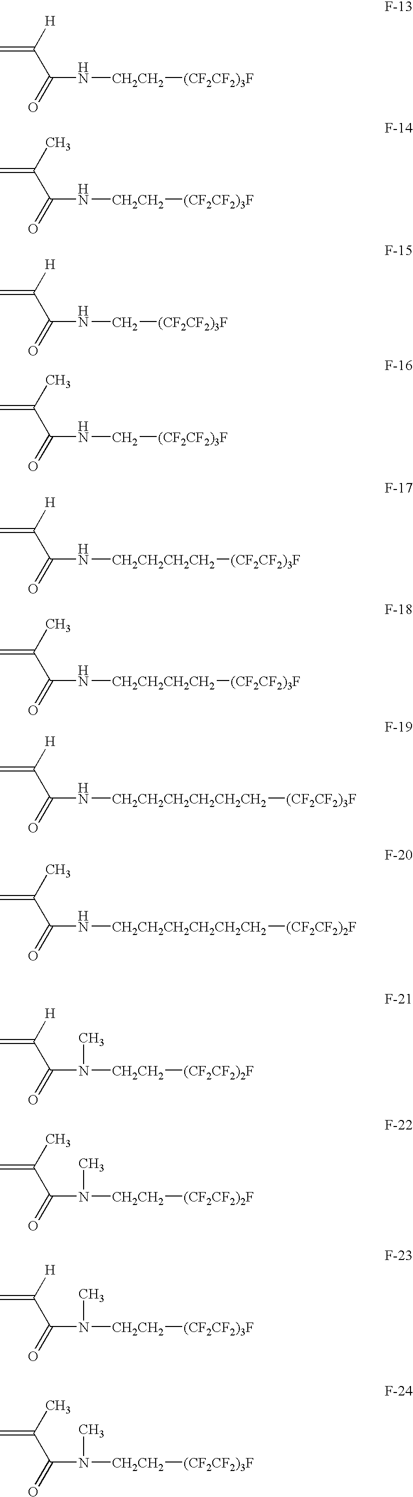

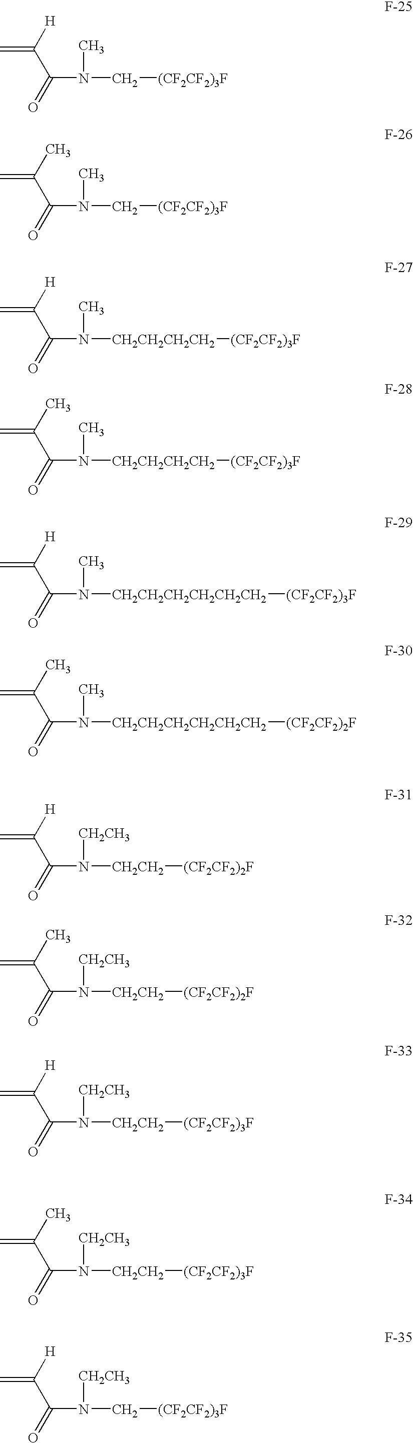

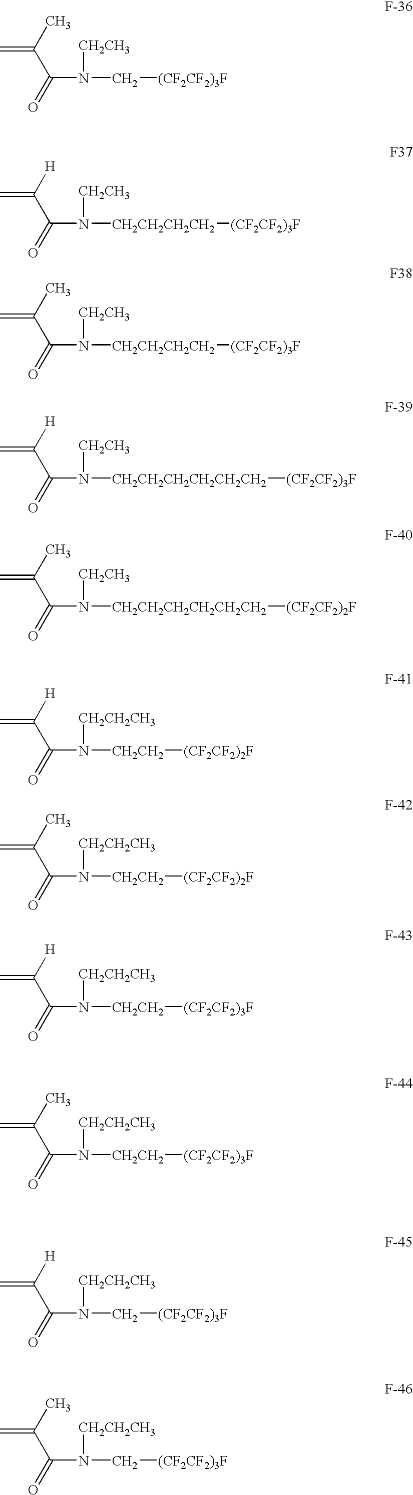

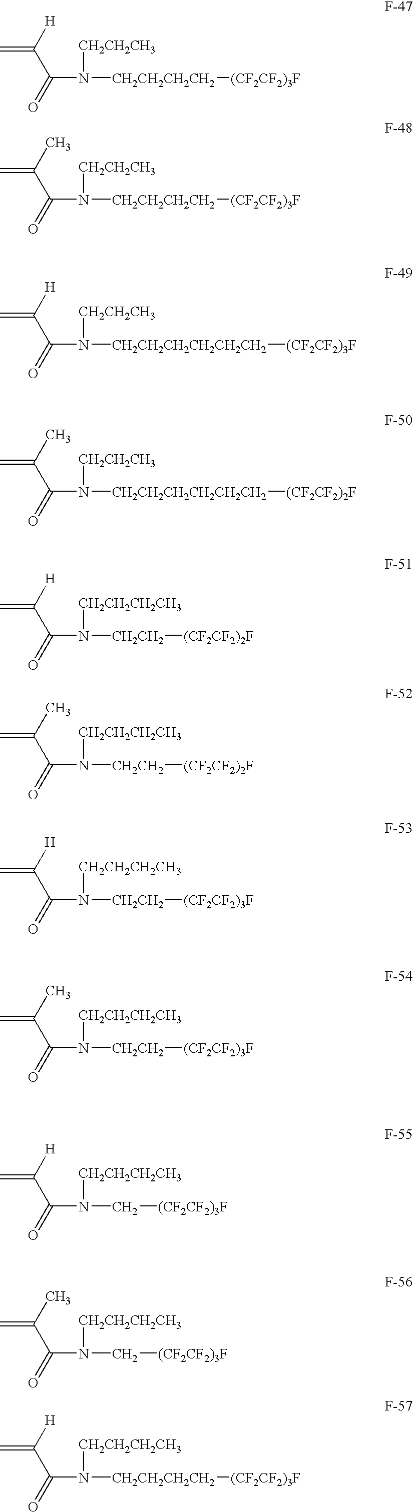

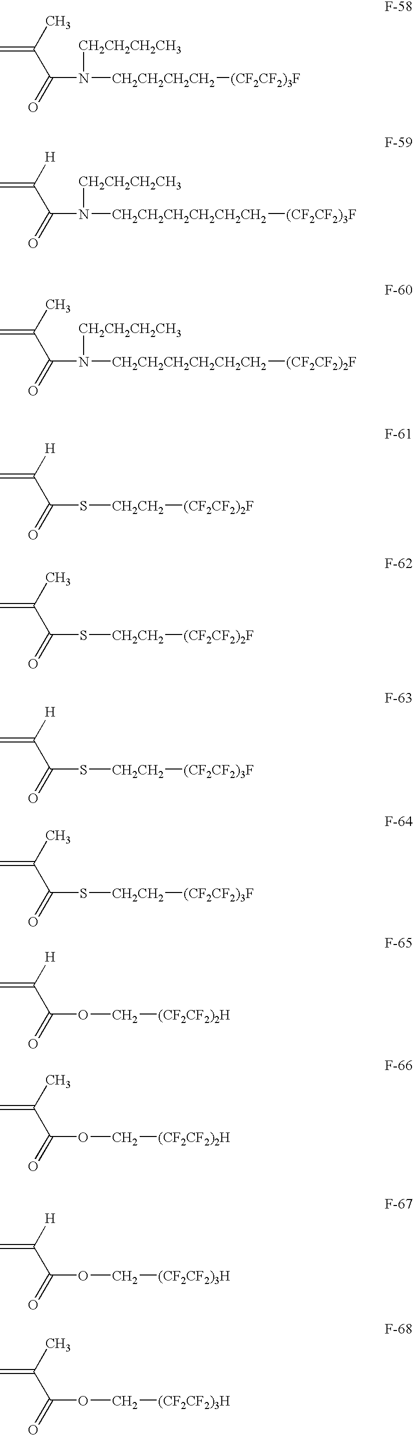

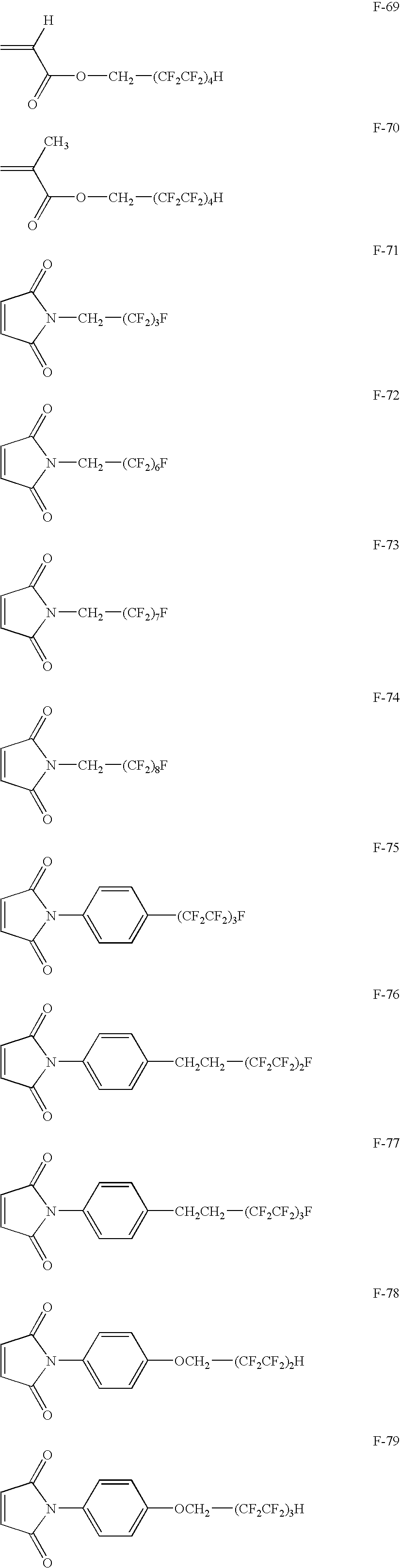

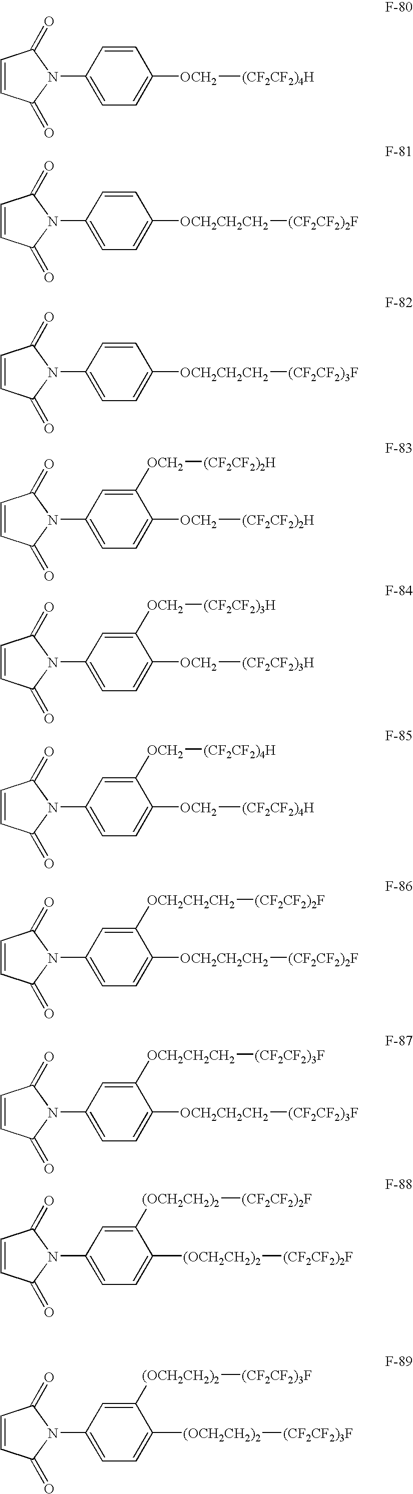

An embodiment of the fluorine-based polymer which can be used in the invention is a copolymer comprising a repeating unit derived from fluoroaliphatic group-containing monomer and a repeating unit containing a hydroxyl group represented by formula (2):

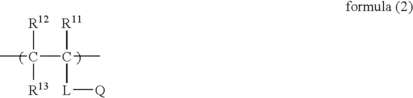

In formula (2), R¹¹, R¹² and R¹³ each independently represents a hydrogen atom or a substituent. Q represents a carboxyl group (—COOH), salt thereof, sulfo group (—SO₃H), salt thereof, phosphonoxy {—OP(═O)(OH)₂}, salt thereof or alkylene group containing poly(alkyleneoxy) group. L represents a divalent connecting group selected from the group consisting of the following connecting groups or a divalent connecting group formed by two or more of the following connecting groups in combination:

### (Group of Connecting Groups)

Single bond, —O—, —CO—, —NR⁴— (in which R⁴ represents a hydrogen atom, alkyl group, aryl group or aralkyl group), —S—, —SO₂—, —P(═O)(OR⁵)— (in which R⁵ represents an alkyl group, aryl group or aralkyl group), alkylene group and arylene group.

In formula (2), R¹¹, R¹² and R¹³ each independently represents a hydrogen atom or substituent. The substituent is preferably one selected from the group consisting of substituents exemplified below.

### (Group of Substituents)

Alkyl group (alkyl group preferably having from 1 to 20 carbon atoms, more preferably from 1 to 12 carbon atoms, particularly preferably from 1 to 8 carbon atoms, e.g., methyl group, ethyl group, isopropyl group, tert-butyl group, n-octyl group, n-decyl group, n-hexadecyl group, cyclopropyl group, cyclopentyl group, cyclohexyl group), alkenyl group (alkenyl group preferably having from 2 to 20 carbon atoms, more preferably from 2 to 12 carbon atoms, particularly preferably from 2 to 8 carbon atoms, e.g., vinyl group, aryl group, 2-butenyl group, 3-pentenyl group), alkinyl group (alkinyl group preferably having from 2 to 20 carbon atoms, more preferably from 2 to 12 carbon atoms, particularly preferably from 2 to 8 carbon atoms, e.g., propargyl group, 3-pentinyl group), aryl group (aryl group preferably having from 6 to 30 carbon atoms, more preferably from 6 to 20 carbon atoms, particularly preferably from 6 to 12 carbon atoms, e.g., phenyl group, p-methylphenyl group, naphthyl group), aralkyl group (aralkyl group preferably having from 7 to 30 carbon atoms, more preferably from 7 to 20 carbon atoms, particularly preferably from 7 to 12 carbon atoms, e.g., benzyl group, phenethyl, 3-phenylpropyl group), substituted or unsubstituted amino group (substituted or unsubstituted amino group preferably having from 0 to 20 carbon atoms, more preferably from 0 to 10 carbon atoms, particularly preferably from 0 to 6 carbon atoms, e.g., unsubstituted amino group, methylamino group, dimethylamino group, diethylamino group, anilino group).

Alkoxy group (alkoxy group preferably having from 1 to 20 carbon atoms, more preferably from 1 to 16 carbon atoms, particularly preferably from 1 to 10 carbon atoms, e.g., methoxy group, ethoxy group, butoxy group), alkoxycarbonyl group (alkoxycarbonyl group preferably having from 2 to 20 carbon atoms, more preferably from 2 to 16 carbon atoms, particularly preferably from 2 to 10 carbon atoms, e.g., methoxycarbonyl group, ethoxycarbonyl group), acyloxy group (acyloxy group preferably having from 2 to 20 carbon atoms, more preferably from 2 to 16 carbon atoms, particularly preferably from 2 to 10 carbon atoms, e.g., acetoxy group, benzoyloxy group), acylamino group (acylamino group preferably having from 2 to 20 carbon atoms, more preferably from 2 to 16 carbon atoms, particularly preferably from 2 to 10 carbon atoms, e.g., acetylamino group, benzoylamino group), alkoxycarbonylamino group (alkoxycarbonylamino group preferably having from 2 to 20 carbon atoms, more preferably from 2 to 16 carbon atoms, particularly preferably from 2 to 12 carbon atoms, e.g., methoxycarbonylamino group), aryloxycarbonylamino group (aryloxycarbonylamino group preferably having 7 to 20 carbon atoms, more preferably 7 to 16 carbon atoms, particularly preferably 7 to 12 carbon atoms, e.g., phenyloxycarbonylamino group), sulfonylamino group (sulfonylamino group preferably having from 1 to 20 carbon atoms, more preferably from 1 to 16 carbon atoms, particularly preferably from 1 to 12 carbon atoms, e.g., methanesulfonylamino group, benzenesulfonylamino group), sulfamoyl group (sulfamoyl group preferably having from 0 to 20 carbon atoms, more preferably from 0 to 16 carbon atoms, particularly preferably from 0 to 12 carbon atoms, e.g., sulfamoyl group, methylsulfamoyl group, dimethylsulfamoyl group, phenylsulfamoyl group), carbamoyl group (carbamoyl group preferably having from 1 to 20 carbon atoms, more preferably from 1 to 16 carbon atoms, particularly preferably from 1 to 12 carbon atoms, e.g., unsubstituted carbamoyl group, methylcarbamoyl group, diethylcarbamoyl group, phenylcarbamoyl group).

Alkylthio group (alkylthio group preferably having from 1 to 20 carbon atoms, more preferably from 1 to 16 carbon atoms, particularly preferably from 1 to 12 carbon atoms, e.g., methylthio group, ethylthio group), arylthio group (arylthio group preferably having from 6 to 20 carbon atoms, more preferably from 6 to 16 carbon atoms, particularly preferably from 6 to 12 carbon atoms, e.g., phenylthio group), sulfonyl group (sulfonyl group preferably having from 1 to 20 carbon atoms, more preferably from 1 to 16 carbon atoms, particularly preferably from 1 to 12 carbon atoms, e.g., mesyl group, tosyl group), sulfinyl group (sulfinyl group preferably having from 1 to 20 carbon atoms, more preferably from 1 to 16 carbon atoms, particularly preferably from 1 to 12 carbon atoms, e.g., methanesulfinyl group, benzenesulfinyl group), ureido group (ureido group preferably having from 1 to 20 carbon atoms, more preferably from 1 to 16 carbon atoms, particularly preferably from 1 to 12 carbon atoms, e.g., unsubstituted ureido group, methylureido group, phenylureido group), phosphoric acid amide group (phosphoric acid amide group preferably having from 1 to 20 carbon atoms, more preferably from 1 to 16 carbon atoms, particularly preferably from 1 to 12 carbon atoms, e.g., diethylphosphoric acid amide group, phenylphosphoric acid amide group), hydroxyl group, mercapto group, halogen atom (e.g., fluorine atom, chlorine atom, bromine atom, iodine atom), cyano group, sulfo group, heterocyclic group (heterocyclic group preferably having from 1 to 30 carbon atoms, more preferably from 1 to 12 carbon atoms, such as heterocyclic group having hetero atoms such as nitrogen atom, oxygen atom and sulfur atom, e.g., imidazolyl group, pyridyl group, quinolyl group, furyl group, piperidyl group, morpholino group, benzooxazolyl group, benzimidazolyl group, benzthiazolyl group), silyl group (silyl group preferably having from 3 to 40 carbon atoms, more preferably from 3 to 30 carbon atoms, particularly preferably from 3 to 24 carbon atoms, e.g., trimethylsilyl group, triphenylsilyl group). These substituents may be further substituted by these substituents. Two or more of these substituents, if used, may be the same or different. If possible, these substituents may be connected to each other to form rings.

R¹¹, R¹² and R¹³ each are preferably a hydrogen atom, an alkyl group, a halogen atom (e.g., fluorine atom, chlorine atom, bromine atom, iodine atom) or a group represented by -L-Q shown below, more preferably a hydrogen atom, a C₁-C₆ alkyl group, a chlorine atom or a group represented by -L-Q, even more preferably a hydrogen atom or a C₁-C₄ alkyl group, particularly preferably a hydrogen atom or a C₁-C₂ alkyl group. Most preferably, R² and R³ each are a hydrogen atom and R¹ is a hydrogen atom or methyl group. Specific examples of the alkyl group employable herein include methyl group, ethyl group, n-propyl group, n-butyl group, and sec-butyl group. The alkyl group may have proper substituents. Examples of the substituents employable herein inchlde halogen atoms, aryl groups, heterocyclic groups, alkoxyl groups, aryloxy groups, alkylthio groups, arylthio groups, acyl groups, hydroxyl groups, acyloxy groups, amino groups, alkoxycarbonyl groups, acylamino groups, oxycarbonyl groups, carbamoyl groups, sulfonyl groups, sulfamoyl groups, sulfonamide groups, sulfolyl groups, and carboxyl groups. The number of carbon atoms in the alkyl group doesn't include the number of carbon atoms in the substituent hereinafter.

L represents a divalent connecting group selected from the group consisting of the aforementioned group of connecting groups or a divalent connecting group formed by combining two or more of these divalent connecting groups. In the aforementioned group of connecting groups, R⁴ in —NR⁴— represents a hydrogen atom, an alkyl group, an aryl group or an aralkyl group, preferably a hydrogen atom or an alkyl group. Further, R⁵ in —PO(OR⁵) represents an alkyl group, an aryl group or an aralkyl group, an aryl group or an aralkyl group, preferably an alkyl group. The number of carbon atoms in the alkyl group, aryl group or aralkyl group represented by R⁴ and R⁵ is the same as described above with reference to “group of substituents”. L preferably includes a single bond, —O—, —CO—, —NR⁴—, —S—, —SO₂—, alkylene group or aryl group, particularly preferably a single bond, —CO—, —O—, —NR⁴—, alkylene group or arylene group, most preferably a single bond. In the case where L include an alkylene group, the number of carbon atoms in the alkylene group is preferably from 1 to 10, more preferably from 1 to 8, particularly preferably from 1 to 6. Specific examples of the alkylene group employable herein include methylene group, ethylene group, trimethylene group, tetrabutylene group, and hexamethylene group. In the case where L represents an arylene group, the number of carbon atoms in the arylene group is preferably from 6 to 24, more preferably from 6 to 18, particularly preferably from 6 to 12. Particularly preferred examples of the arylene group include phenylene group, and naphthalene group. In the case where L is a divalent connecting group obtained by combining an alkylene group and an arylene group, the number of carbon atoms in the aralkylene group is preferably from 7 to 34, more preferably from 7 to 26, particularly preferably from 7 to 16. Specific examples of particularly desirable aralkylene group include phenylene methylene group, phenylene ethylene group, and methylene phenylene group. The groups exemplified as L may have proper substituents. Examples of these substituents include those exemplified above as substituents on R¹¹ to R¹³.

Specific configurations of L will be exemplified below, the invention is not limited thereto.

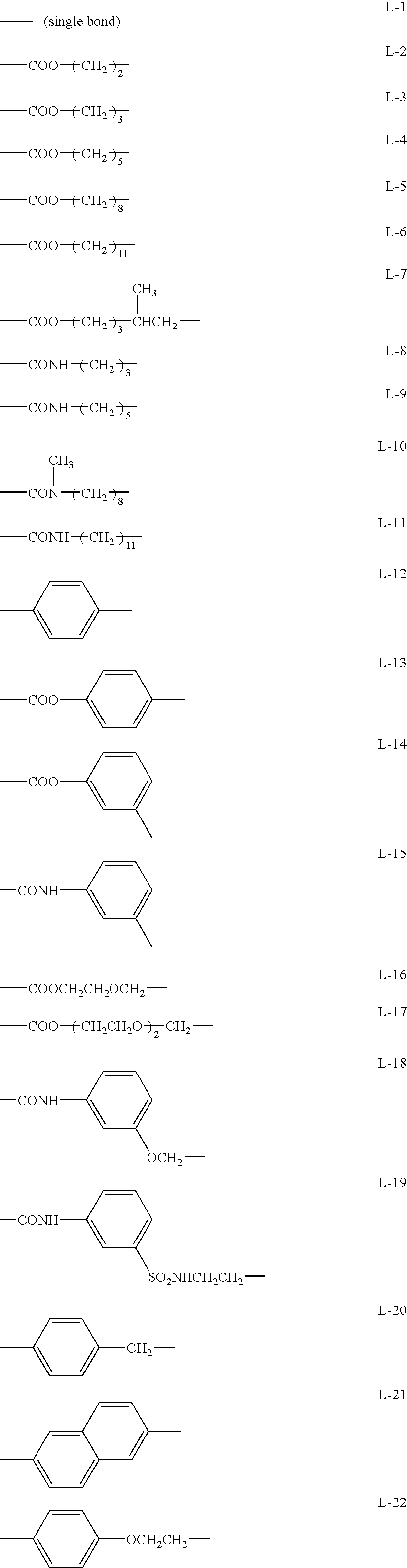

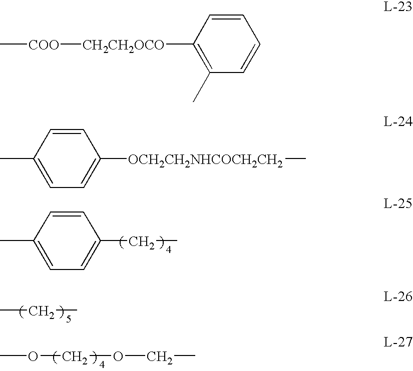

In formula (2), Q represents a carboxyl group, carboxyl group salt (e.g., lithium salt, sodium salt, potassium salt, ammonium salt (e.g., ammonium, tetramethyl ammonium, trimethyl-2-hydroxyethyl ammonium, tetrabutyl ammonium, trimethylbenzyl ammonium, dimethylphenyl ammonium), pyridinium salt), sulfo group, sulfo group salt (Examples of the cation which forms the salt include those described above with reference to carboxyl group), phosphonoxy group, phosphonoxy group salt (Examples of the cation which forms the salt include those described above with reference to carboxyl group) or alkyl group containing a poly(alkyleneoxy) group. The poly(alkyleneoxy) group is represented by (OR)ₓ in which R is preferably a C₂-C₄ alkylene group such as —CH₂CH₂—, —CH₂CH₂CH₂—, —CH(CH₃)CH₂— or —CH(CH₃)CH(CH₃)—.

The oxyalkylene unit in the aforementioned poly (oxyalkylene) group may be the same as that in the poly (oxypropylene) or may have two or more different oxyalkylenes irregularly distributed therein or may be a straight-chain or branched oxypropylene or oxyethylene unit or may be in the form of block of straight-chain or branched oxypropylene units or block of oxyethylene units.

This poly(oxyalkylene) chain may include those connected with one or more connecting groups (e.g., —CONH-Ph-NHCO—, —S—, etc. (in which Ph represents a phenylene group)). In the case where the connecting group has a valency of 3 or more, a branched oxyalkylene unit can be obtained.

Further, in the case where a copolymer containing a polymerizable unit having a poly(oxyalkylene) group is used in the invention, the molecular weight of the poly(oxyalkylene) group is preferably from 250 to 3,000.

The poly(oxyalkylene)acrylate and methacrylate can be produced by reacting a commercially available hydroxypoly(oxyalkylene) material, e.g., Pluronic (produced by ADEKA CORPORATION), Adeka Polyether (produced by ADEKA CORPORATION), Carbowax (produced by Carbowax (produced by Glyco Products Co., Ltd.), Toriton (produced by Rohm & Haas Inc.), P. E. G (produced by DAI-ICHI KOCGYO SEIYAKU CO., LTD.) with an acrylic acid, methacrylic acid, acryl chloride, methacryl chloride, acryl anhydride or the like by any known method. Alternatively, a poly(oxyalkylene)diacrylate or the like produced by any known method may be used.

Specific examples of the monomer corresponding to formula (2) which can be used in the production of the fluorine-based polymer to be used in the invention will be given below, but the invention is not limited thereto. A poly(alkyleneoxy) often occurs in the form of those having different polymerization degrees X. In the compounds exemplified as specific examples below, too, the polymerization degree is represented by an integer close to the averaged polymerization degree.

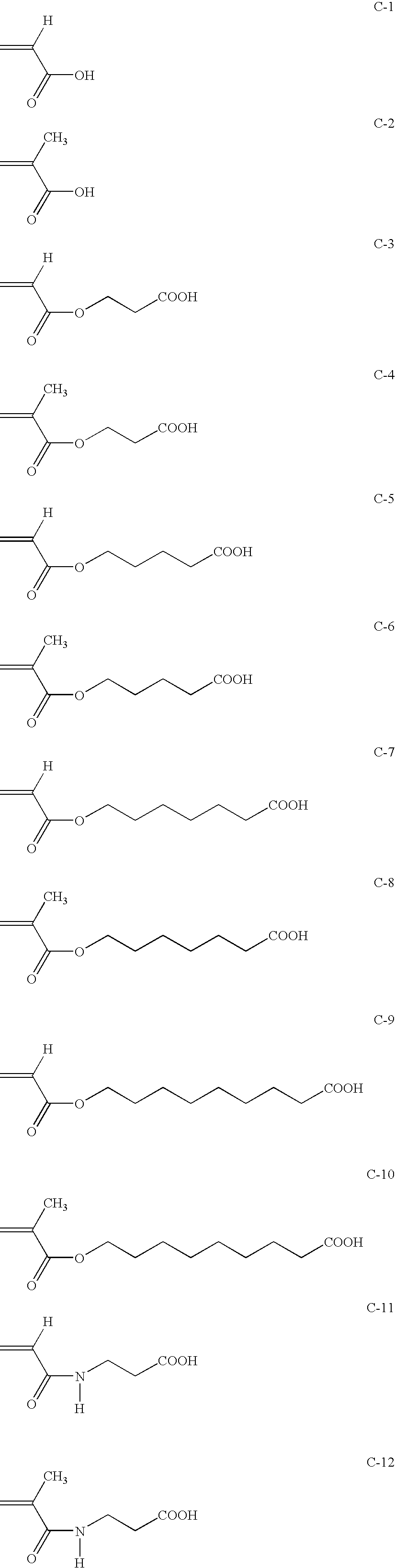

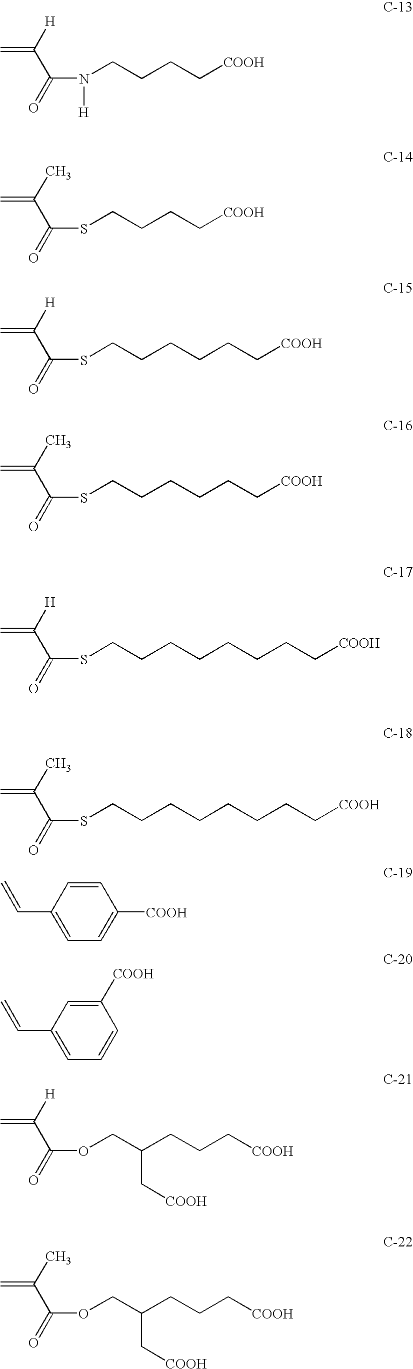

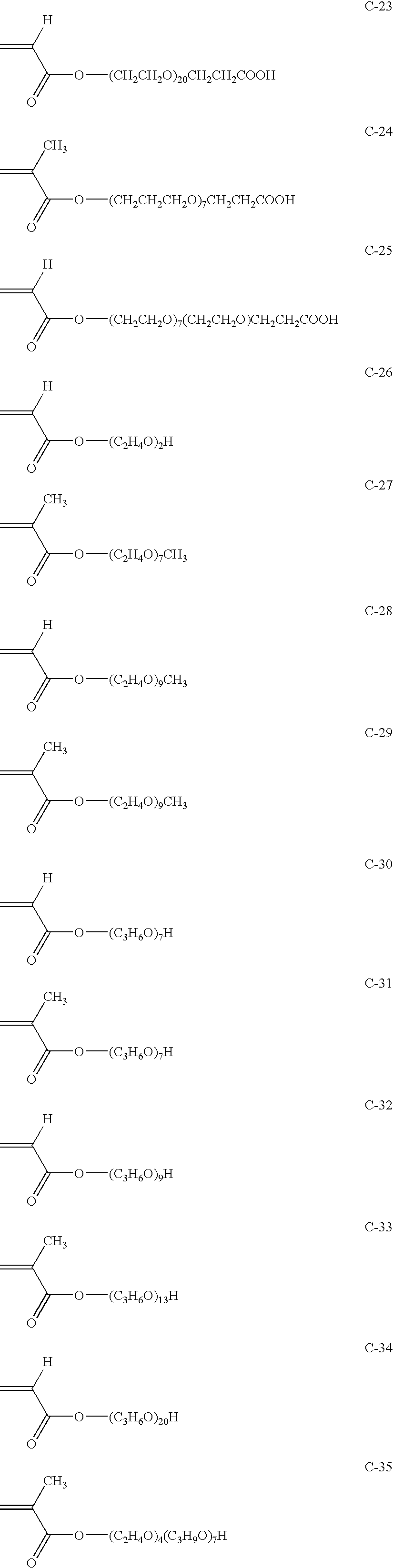

The aforementioned fluorine-based polymer may contain one or more of the repeating units represented by formula (2). The aforementioned fluorine-based polymer may contain one or more of repeating units other than the aforementioned repeating units The content of the repeating units represented by formula (2) or the repeating units other than the repeating units represented by formula (2) in the fluorine-based polymer is preferably 95% by mass or less, more preferably 90% by mass or less, even more preferably 70% by mass or less based on the total amount of monomers constituting the polymer. The aforementioned other repeating units are not specifically limited. Preferred examples of the other repeating units include those derived from ordinary radical-polymerizable monomers. Specific examples of the monomers from which the other repeating units are derived will be given below. The aforementioned fluorine-based polymer may contain repeating units derived from one or more monomers selected from the group consisting of the following monomer groups.

### Group of Monomers

### (1) Alkenes

Ethylene, propylene, 1-butene, isobutene, 1-hexene, 1-dodecene, 1-octadecene, 1-eicocene, hexafluoropropene, vinylidene fluoride, chlorotrifluoroethylene, 3,3,3-trifluoropropylene, tetrafluoroethylene, vinyl chloride, vinylidene chloride, etc.,

### (2) Dienes

1,3-Butadiene, isoprene, 1,3-pentadiene, 2-ethyl-1,3-butadiene, 2-n-propyl-1,3-butadiene, 2,3-dimethyl-1,3-butadiene, 2-methyl-1,3-pentadiene, 1-phenyl-1,3-butadiene, 1-α-naphthyl-1,3-butadiene, 1-β-naphthyl-1,3-butadiene, 2-chloro-1,3-butadiene, 1-bromo-1,3-butadiene, 1-chlorobutadiene, 2-fluoro-1,3-butadiene, 2,3-dichloro-1,3-butadiene, 1,1,2-trichloro-1,3-butadiene, and 2-cyano-1,3-butadiene, 1,4-divinyl cyclohexane, etc.;

(3) Derivatives of α,β-unsaturated carboxylic acids

### (3a) Alkyl Acrylates

Methyl acrylate, ethyl acrylate, n-propyl acrylate, isopropyl acrylate, n-butyl acrylate, isobutyl acrylate, sec-butyl acrylate, tert-butyl acrylate, amyl acrylate, n-hexyl acrylate, cyclohexyl acrylate, 2-ethylhexyl acrylate, n-octyl acrylate, tert-octyl acrylate, dodecyl acrylate, phenyl acrylate, benzyl acrylate, benzyl acrylate, 2-chloroethyl acrylate, 2-bromoethyl acrylate, 4-chlorobutyl acrylate, 2-cyanoethyl acrylate, 2-acetoxyethyl acrylate, methoxybenzyl acrylate, 2-chlorocyclohexyl acrylate, furfiuryl acrylate, tetrahydrofurfuryl acrylate, 2-methoxyethyl acrylate, ω-methoxypolyethylene glycol acrylate (number of added moles in polyoxyethylene: n=2 to 100), 3-methoxybutyl acrylate, 2-ethoxyethyl acrylate, 2-butoxyethyl acrylate, 2-(2-butoxyethoxy)ethyl acrylate, 1-bromo-2-methoxyethyl acrylate, 1,1-dichloro-2-ethoxyethyl acrylate, glycidyl acrylate, etc.);

### (3b) Alkyl Methacrylates

Methyl methacrylate, ethyl methacrylate, n-propyl methacrylate, isopropyl methacrylate, n-butyl methacrylate, isobutyl methacrylate, sec-butyl methacrylate, tert-butyl methacrylate, amyl methacrylate, n-hexyl methacrylate, cyclohexyl methacrylate, 2-ethylhexyl methacrylate, n-octyl methacrylate, stearyl methacrylate, benzyl methacrylate, phenyl methacrylate, allyl methacrylate, furfuryl methacrylate, tetrahydrofurfuryl methacrylate, cresyl methacrylate, naphthyl methacrylate, 2-methoxyethyl methacrylate, 3-methoxybutyl methacrylate, ω-methoxy polyethylene glycol methacrylate (number of added moles in polyoxyethylene: n=2 to 100), 2-acetoxyethyl methacrylate, 2-ethoxyethyl methacrylate, 2-butoxyethyl methacrylate, 2-(2-butoxyethoxy)ethyl methacrylate, glycidyl methacrylate, 3-trimethoxysilylpropyl methacrylate, allyl methacrylate, 2-isochyanatoethyl methacrylate, etc.;

### (3c) Unsaturated Polyvalent Carboxyl Acid Diesters

Dimethyl maleate, dibutyl maleate, dimethyl itaconate, dibutyl itaconate, dibutyl crotonate, dihexyl crotonate, diethyl fumarate, dimethyl fumarate, etc.;

(3e) α,β-Unsaturated carboxylic acid amides

N,N-dimethyl acrylamide, N,N-diethyl acrylamide, N-n-propyl acrylamide, N-tert-butyl arylamide, N-tert-octyl methacrylamide, N-cyclohexyl acrylamide, N-phenyl acrylamide, N-(2-acetoacetoxyethyl)acrylamide, N-benzyl acrylamide, N-acryloyl morpholine, diacetone acrylamide, N-methyl maleimide, etc.;

### (4) Unsaturated Nitriles

Acrylonitrile, methacrylonitrile, etc.;

### (5) Styrenes and Derivatives Thereof.

Styrene, vinyl toluene, ethyl styrene, p-tert-butyl styrene, methyl p-vinylbenzoate, α-methyl styrene, p-chloromethyl styrene, vinyl naphthalene, p-methoxy styrene, p-hydroxymethyl styrene, p-acetoxystyrene, etc.;

### (6) Vinyl Esters

Vinyl acetate, vinyl propionate, vinyl butyrate, vinyl isobutyrate, vinyl salicylate, vinyl chloroacetate, vinyl methoxyacetate, vinyl phenylacetate, etc.;

### (7) Vinyl Ethers

Methyl vinyl ether, ethyl vinyl ether, n-propylvinyl ether, isopropylvinyl ether, tert-butyl vinyl ether, n-pentylvinyl ether, n-hexylvinyl ether, n-octylvinyl ether, n-dodecylvinyl ether, n-eicosilvinyl ether, 2-ethylhexylvinyl ether, cyclohexylvinyl ether, fluorobutylvinyl ether, fluorobutoxyethylvinyl ether, etc.; and

### (8) Other Polymerizable Monomers

N-vinylpyrrolidone, methyl vinyl ketone, phenyl vinyl ketone, methoxyethyl vinyl ketone, 2-vinyloxazoline, 2-isopropenyloxazoline, etc.

As the monomer from which the other repeating units are derived there is preferably used one represented by formula (3).

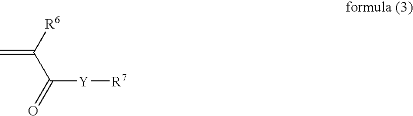

In formula (3), R⁶ represents a hydrogen atom or methyl group, Y represents a divalent connecting group, and R⁷ represents a C₁-C₂₀ straight-chain, branched or cyclic alkyl group which may have substituents. The divalent connecting group represented by Y is preferably an oxygen atom, sulfur atom or —N(R⁵)— in which R⁵ is preferably a hydrogen atom or a C₁-C₄ alkyl group such as methyl, ethyl, propyl and butyl. R⁵ is more preferably a hydrogen atom or methyl Y is particularly preferably an oxygen atom, —NH— or —N(CH₃)—.

As the C₁-C₂₀ straight-chain, branched or cyclic alkyl group represented by R⁷ there is preferably used a methyl group, ethyl group, propyl group, butyl group, pentyl group, hexyl group, heptyl group, octyl group, nonyl group, decyl group, undecyl group, dodecyl group, tridecyl group, tetradecyl group, heptadecyl group, octadecyl group or eicosanyl group which may be straight-chain or branched, monocyclic cycloalkyl group such as cyclohexyl group and cycloheptyl group or polycyclic cycloalkyl group such as bicycloheptyl group, bicycloheptyl group, tricycloundecyl group, tetracyclododecyl group, adamanthyl group, norbornyl group and tetracyclodecyl group.

Examples of the substituents on the alkyl group represented by R⁷ include hydroxyl groups, alkylcarbonyl groups, arylcarbonyl groups, alkylcarbonyloxy groups, carboxyl groups, alkylether groups, arylether groups, halogen atoms such as fluorine atom, chlorine atom and bromine atom, nitro groups, cyano groups, and amino groups, but the invention is not limited thereto.

Particularly preferred among the monomers represented by formula (3) are alkyl (meth)acrylates or poly(alkyleneoxy) (meth)acrylates.

Specific examples of the monomer represented by formula (3) will be given below, the invention is not limited thereto.

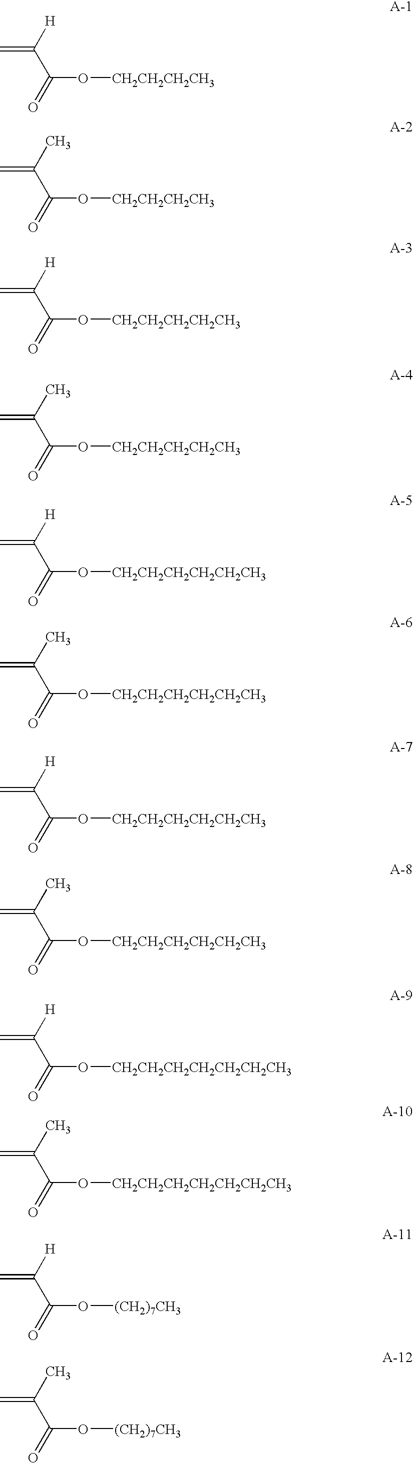

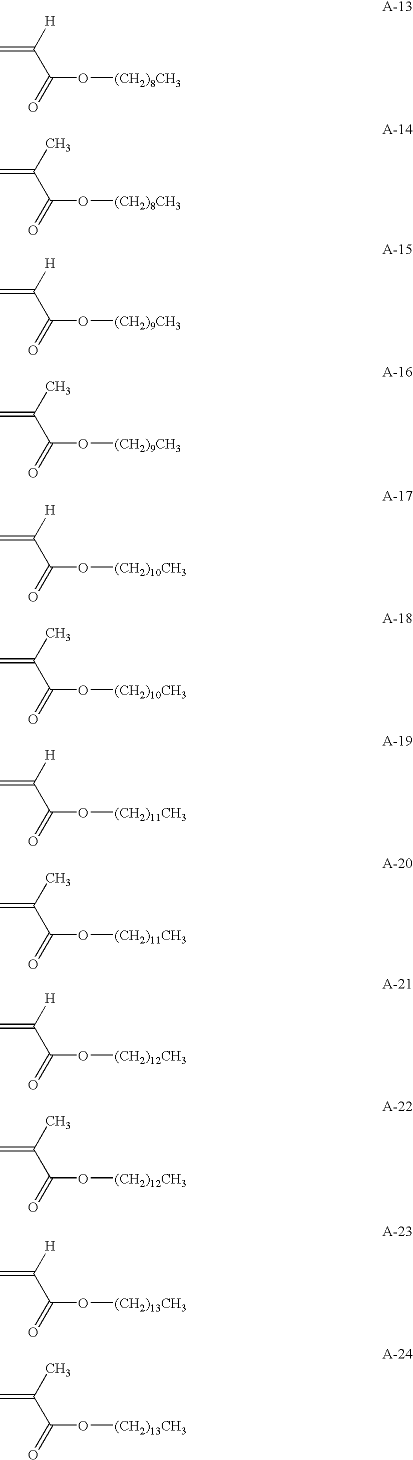

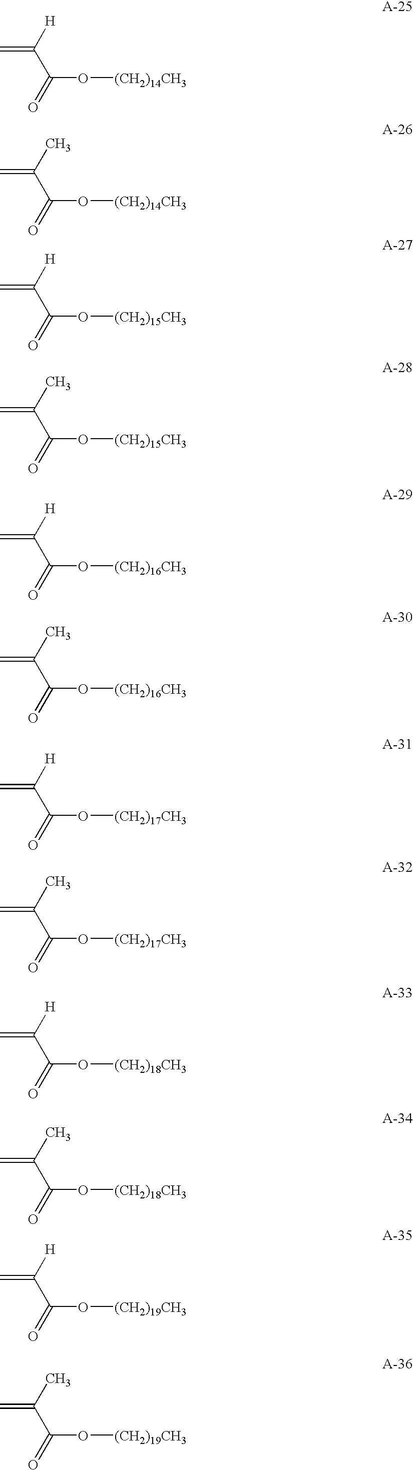

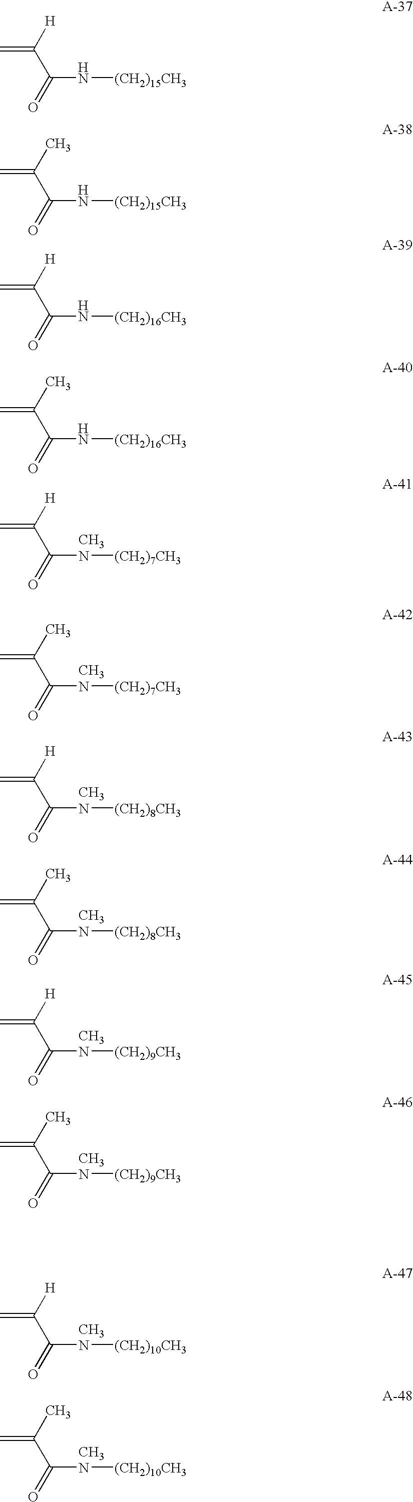

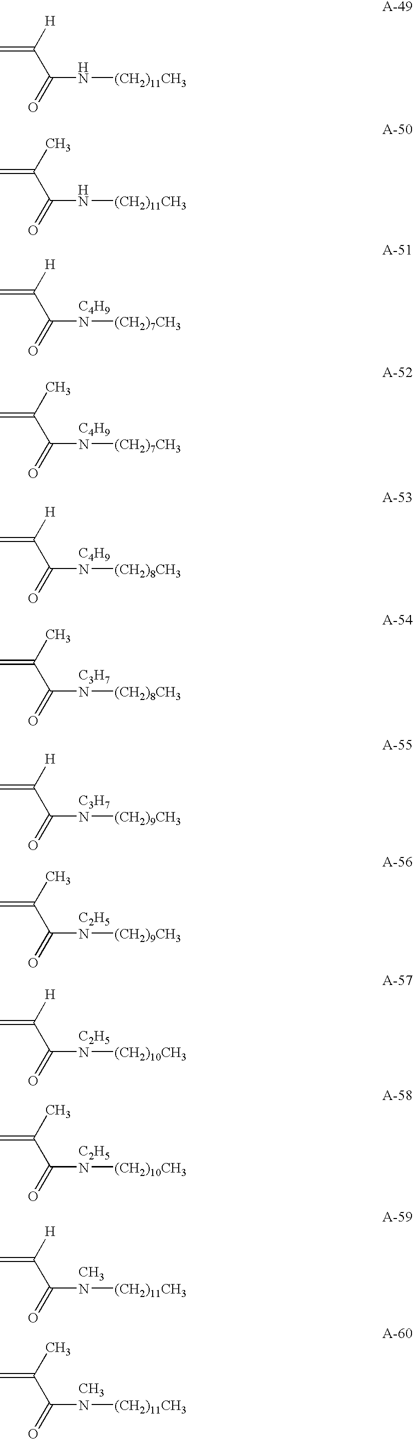

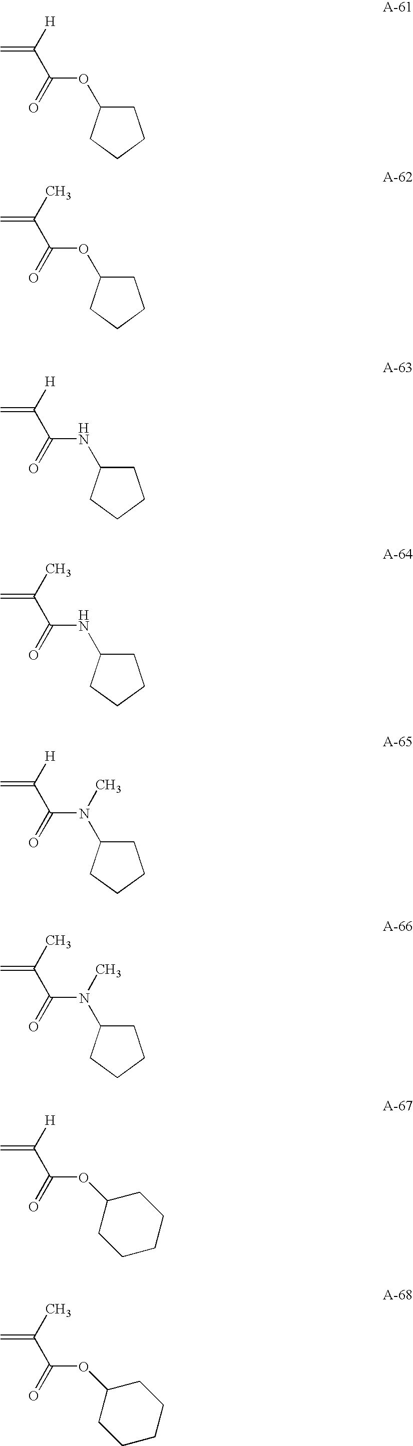

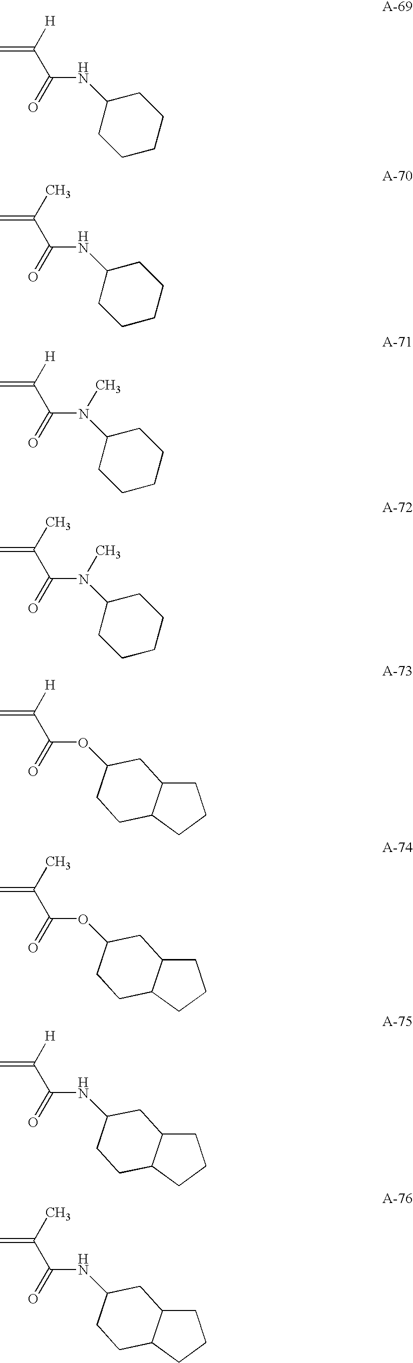

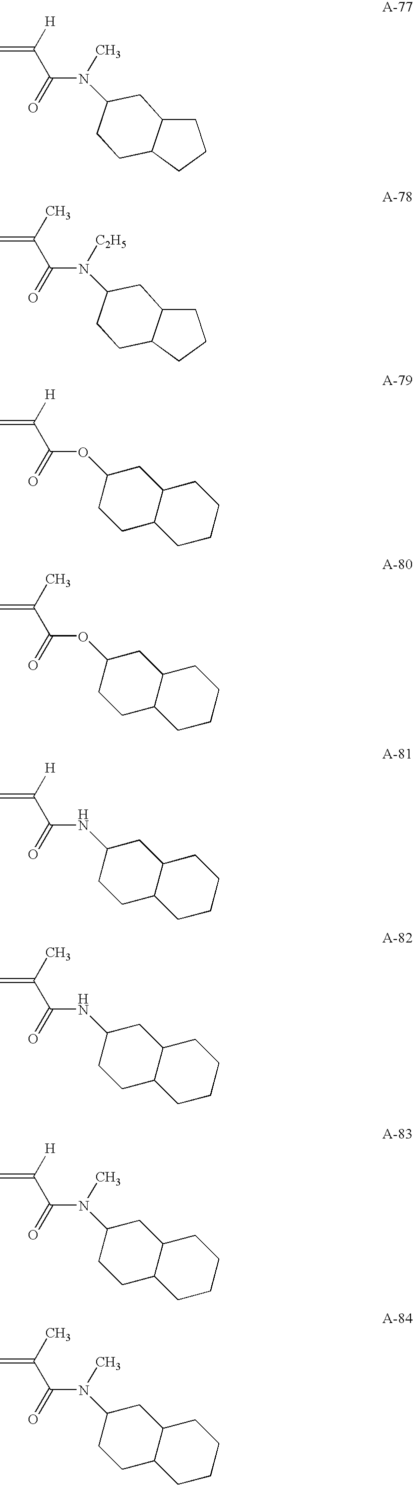

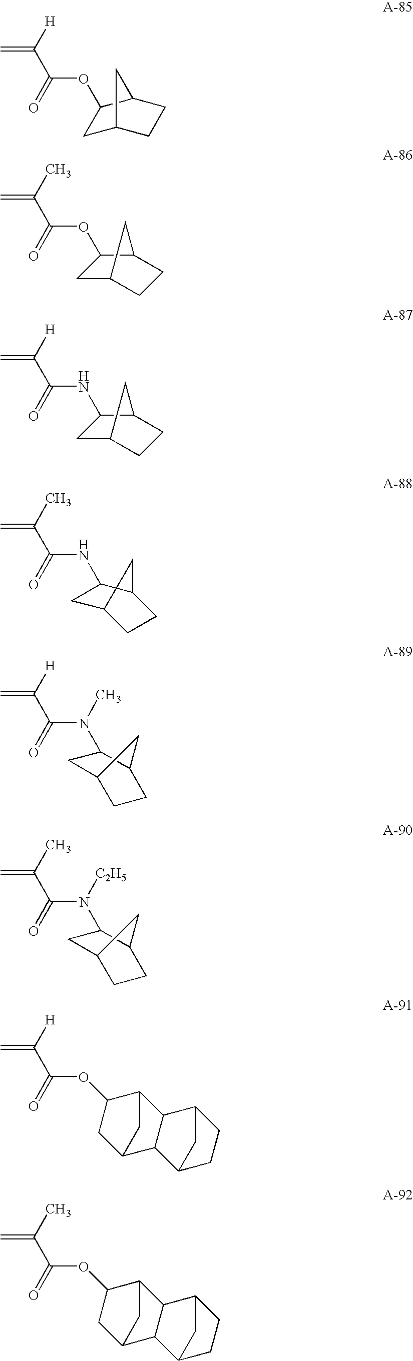

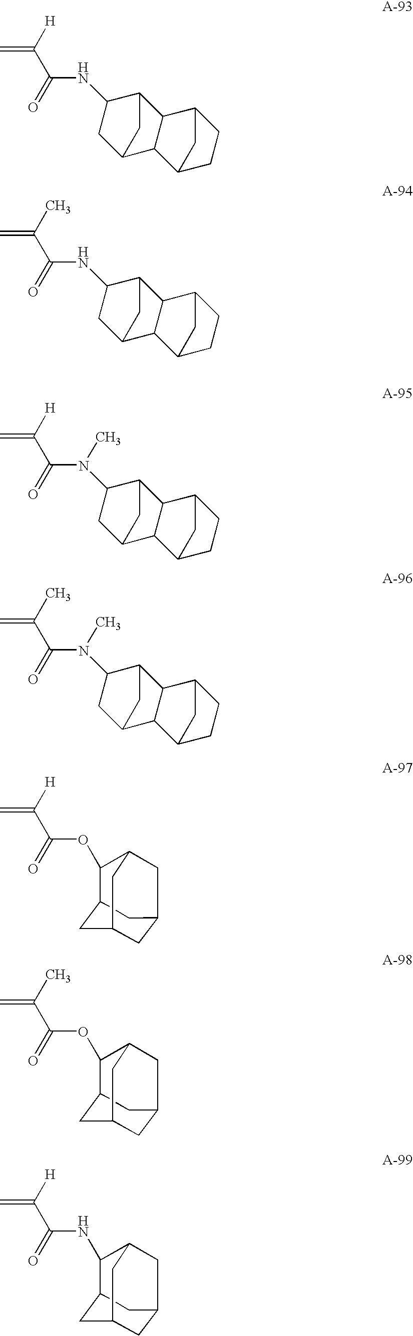

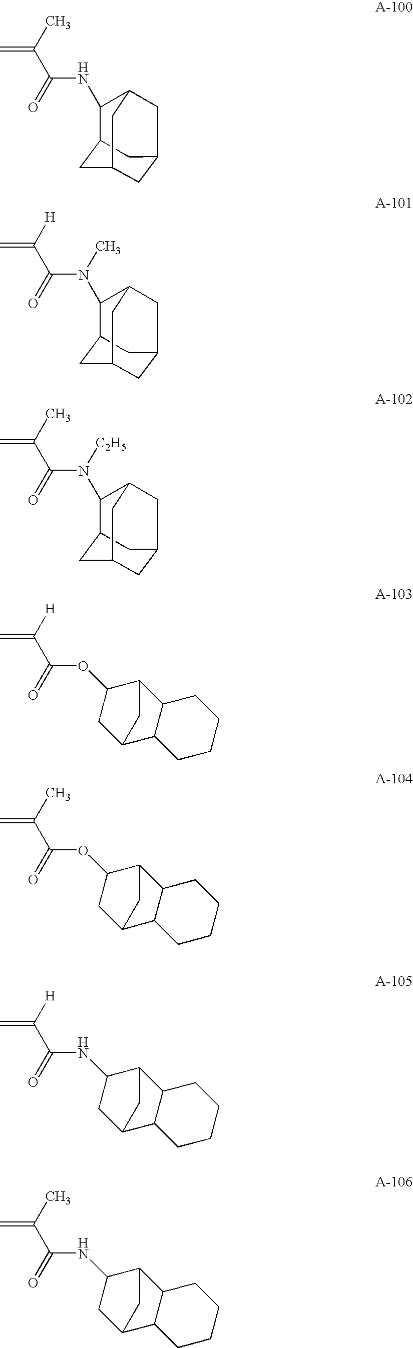

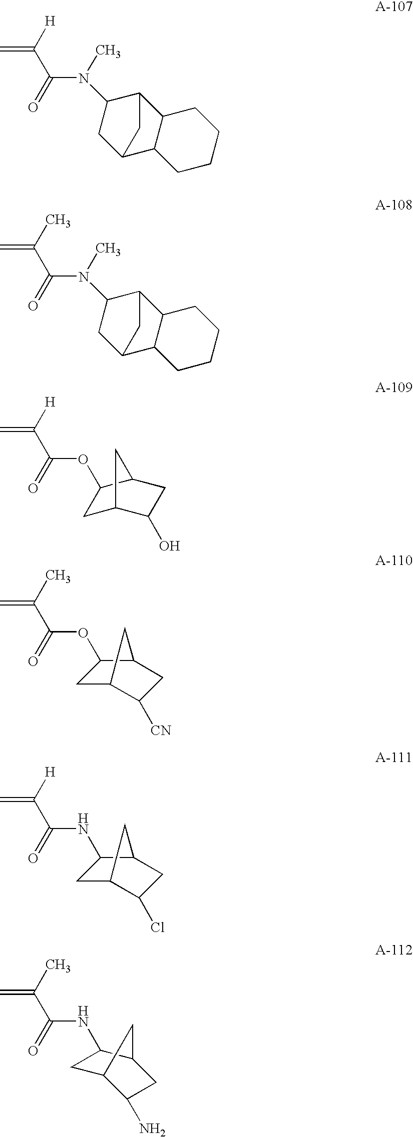

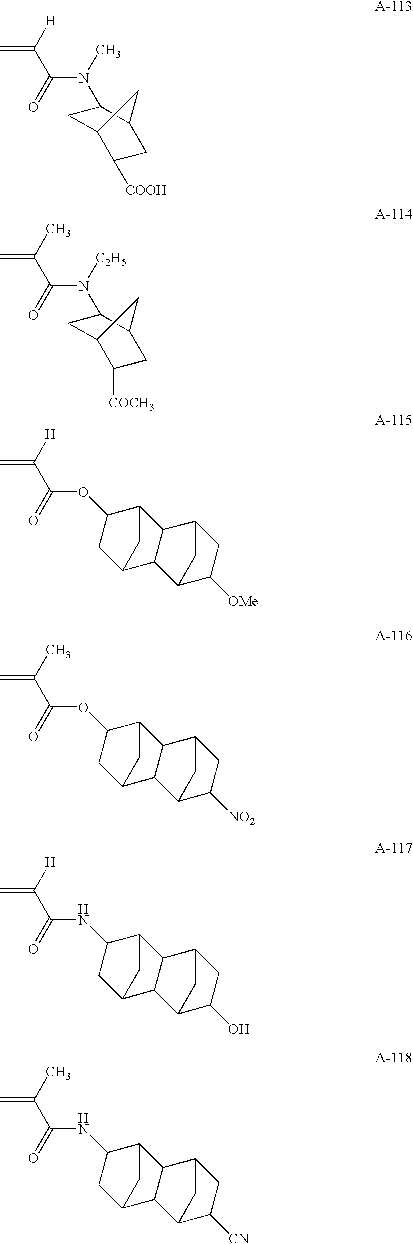

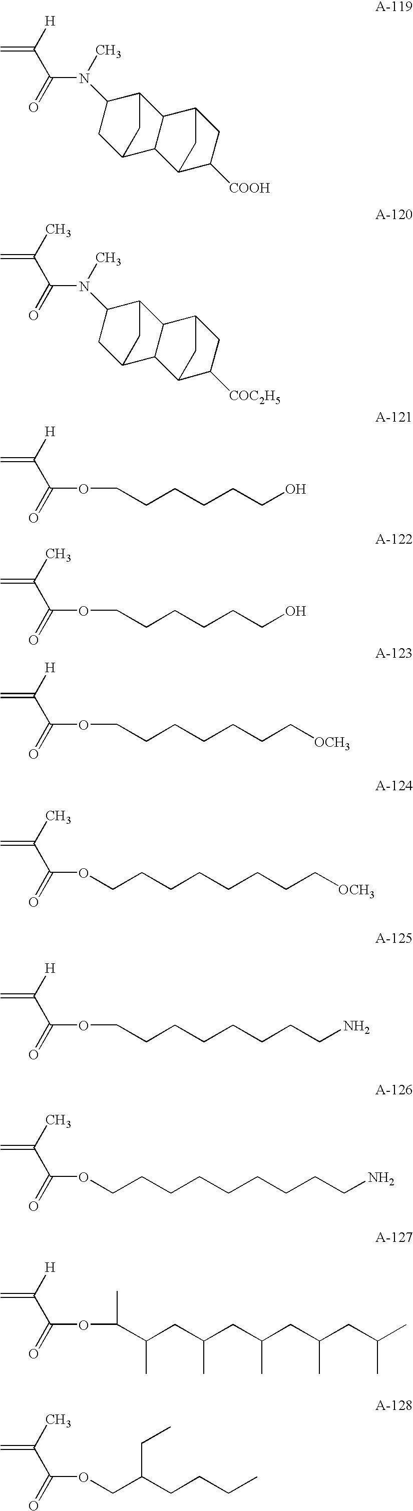

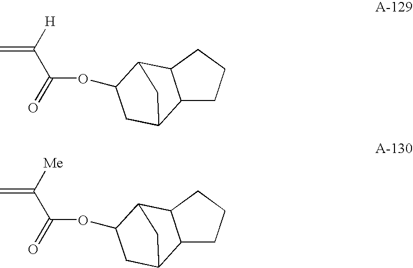

The incorporation of at least two of the aforementioned fluorine-based polymers in the optically anisotropic layer makes it possible to effect the control over the liquid crystal compound and the elimination of unevenness independently. As a result, the desired viewing angle properties and desired surface conditions can be attained at the same time.

The preferable amount of the fluoroaliphatic group-containing monomer in the aforementioned fluorine-based polymer depends on the thickness of the optically anisotropic layer and is generally 5% by mass or more based on the total amount of the monomers constituting the polymer.

The weight-average molecular weight of the aforementioned fluorine-based polymer to be used is preferably from not smaller than 1,000 to not greater than 1,000,000, more preferably from not smaller than 1,000 to not greater than 500,000, even more preferably from not smaller than 1,000 to not greater than 100,000. The weight-average molecular weight of the fluorine-based polymer can be measured by gel permeation chromatography (GPC) in equivalence of polystyrene (PS).

The polymerization method for the production of the aforementioned fluorine-based polymer is not specifically limited but may be a cationic polymerization or radical polymerization method involving the utilization of vinyl group or a polymerization method such as anionic polymerization. Particularly preferred among these polymerization methods is radical polymerization method from the standpoint of general-purpose properties. As a radical polymerization initiator there may be used any known compound such as radical heat polymerization initiator or radical photopolymerization initiator, particularly preferably radical heat polymerization initiator. The term “radical heat polymerization initiator” as used herein is meant to indicate a compound which generates radicals when heated to a temperature of not lower than its decomposition temperature. Examples of such a radical heat polymerization initiator employable herein include diacyl peroxides (e.g., acetyl peroxide, benzoyl peroxide), ketone peroxides (e.g., methyl ethyl peroxide, cyclohexanone peroxide), hydroperoxides (hydrogen peroxide, tert-butyl hydroperoxide, cumene hydroperoxide), dialkyl peroxides (di-tert-butyl peroxide, dicumyl peroxide, dilauroyl peroxide), peroxyesters (e.g., tert-butylperoxy acetate, tert-butylperoxy pivalate), azo-based compounds (e.g., azobisisobutylonitrile, azobisisovaleronitrile), and persulfates (e.g., ammonium persulfate, sodium persulfate, potassium persulfate). These radical heat polymerization initiators may be used singly or in combination of two or more thereof.

The radical polymerization method to be used herein is not specifically limited. Emulsion polymerization method, suspension polymerization method, bulk polymerization method, solution polymerization or the like may be employed in the invention. The solution polymerization method, which is one of typical radical polymerization methods, will be further described below. The outline of the other polymerization methods are similar to that of the solution polymerization method. For the details of these polymerization methods, reference can be made to “Kobunshi Kagaku Jikkenho (Polymer Scientific Experiment Methods)”. The Society of Polymer Science, Japan, TOKYO KAGAKU DOZIN CO., LTD., 1981.

In order to effect solution polymerization, an organic solvent is used. Such an organic solvent can be arbitrarily selected so far as the aim and effect of the invention cannot be impaired. The organic solvent is normally an organic compound having a boiling point of from 50° C. to 200° C. at the atmosphere, preferably an organic compound capable of dissolving the various constituents uniformly therein. Preferred examples of the organic solvent employable herein include alcohols such as isopropanol and butanol, ethers such as dibutyl ether, ethylene glycol dimethyl ether, tetrahydrofurane and dioxane, ketones such as acetone, methyl ethyl ketone, methyl isobutyl ketone and cyclohexanone, esters such as ethyl acetate, butyl acetate, amyl acetate and γ-butyrolactone, and aromatic hydrocarbons such as benzene, toluene and xylene. These organic solvents may be used singly or in combination of two or more thereof. Further, a water-mixed organic solvent comprising the organic solvent mixed with water may be used from the standpoint of capability of dissolving the monomer or the polymer thus produced therein.

Further, the solution polymerization conditions, too, are not specifically limited. For example, however, the monomers are preferably heated to a temperature of from 50° C. to 200° C. for 10 minutes to 30 hours. Further, purge with inactive gas is preferably effected before initiation of solution polymerization, not to mention during solution polymerization, so that the radicals generated cannot be deactivated. As such an inactive gas there is preferably used nitrogen gas.

In order to obtain the aforementioned fluorine-based polymer in a desired range of molecular weight, radical polymerization method using a chain transfer agent is particularly effective. As such a chain transfer agent there may be used any of mercaptans (e.g., octyl mercaptan, decyl mercaptan, dodecyl mercaptan, tert-dodecyl mercaptan, octadecyl mercaptan, thiophenol, p-nonylthiophenol), polyhalogenated alkyls (e.g., carbon tetrachloride, chloroform, 1,1,1-trichloroethane, 1,1,1-tribromooctane), and low activity monomers (e.g., a-methyl styrene, a-methyl styrene dimer). Preferred among these chain transfer agents are C₄-C₁₆ mercaptans. The amount of the chain transfer agent to be used is remarkably affected by the activity of the chain transfer agent, the combination of monomers, the polymerization conditions, etc, and thus needs to be precisely controlled but is normally from about 0.01 mol-% to 50 mol-%, preferably from about 0.05 mol-% to 30 mol-%, particularly preferably from 0.08 mol-% to 25 mol-% based on the total moles of the monomers used. These chain transfer agents may be present in the reaction system at the same time with the monomer to be controlled in polymerization degree during the polymerization process. The method for addition of these chain transfer agents is not specifically limited. These chain transfer agents may be added in the form of solution in monomer or may be added separately of monomer.

Specific examples of the fluoroaliphatic group-containing polymer which is preferably used in the invention will be given below, but the invention is not limited thereto. The numerical value with these general formulas each represent the weight percentage indicating the composition ratio of the various monomers. Mw is the weight-average molecular weight in PS equivalence measured by GPC. The numerical values a, b, c and d each indicate the weight ratio.

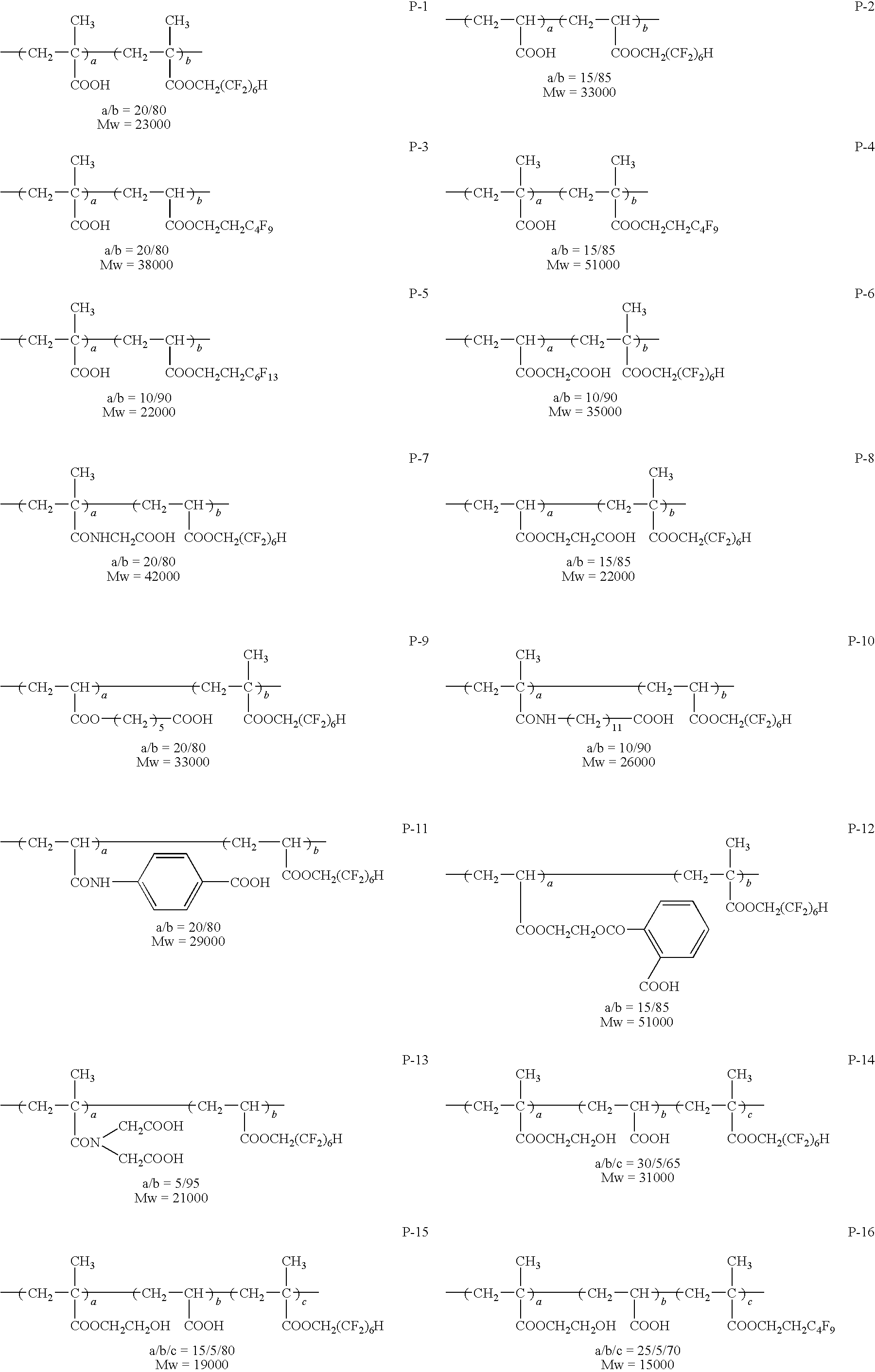

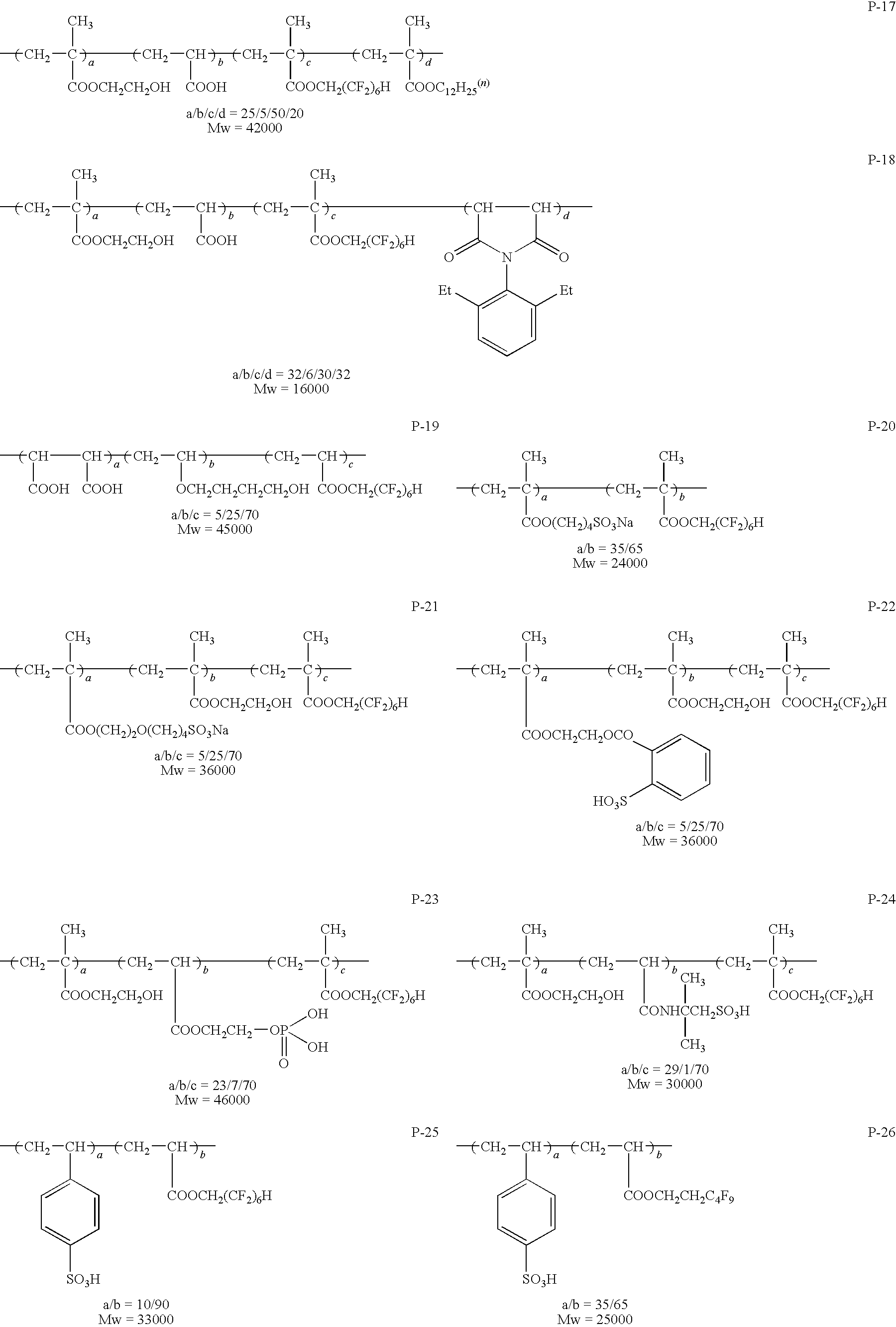

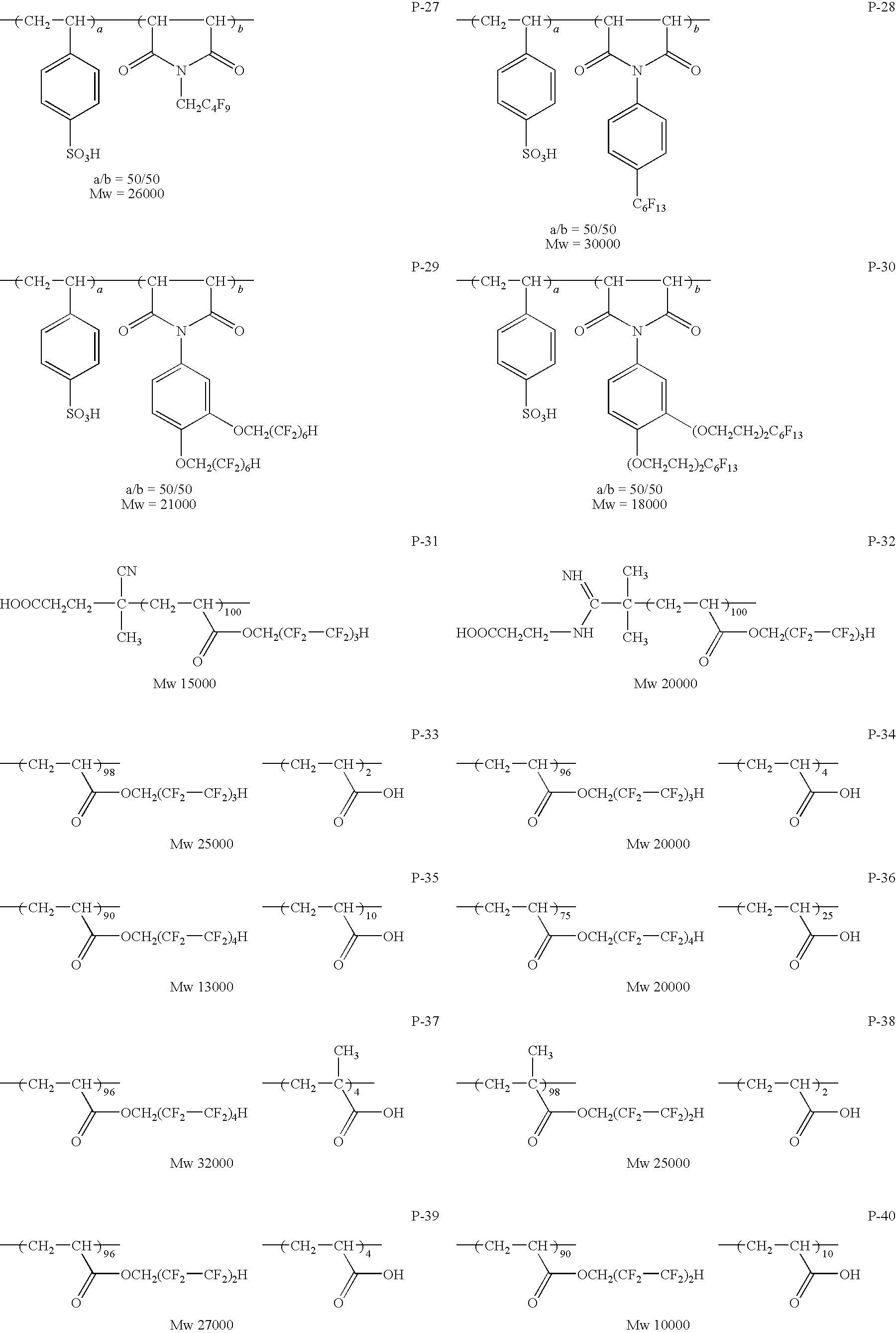

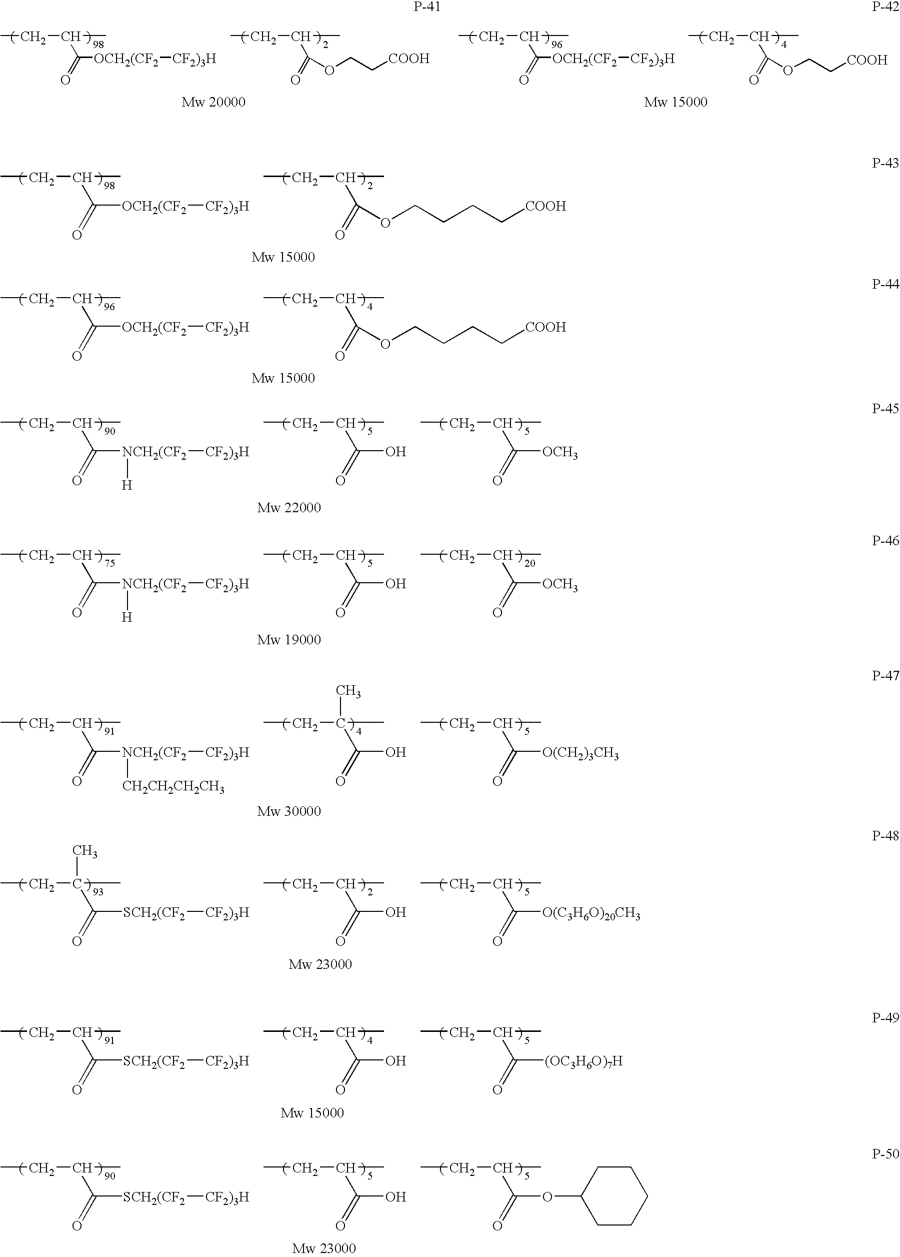

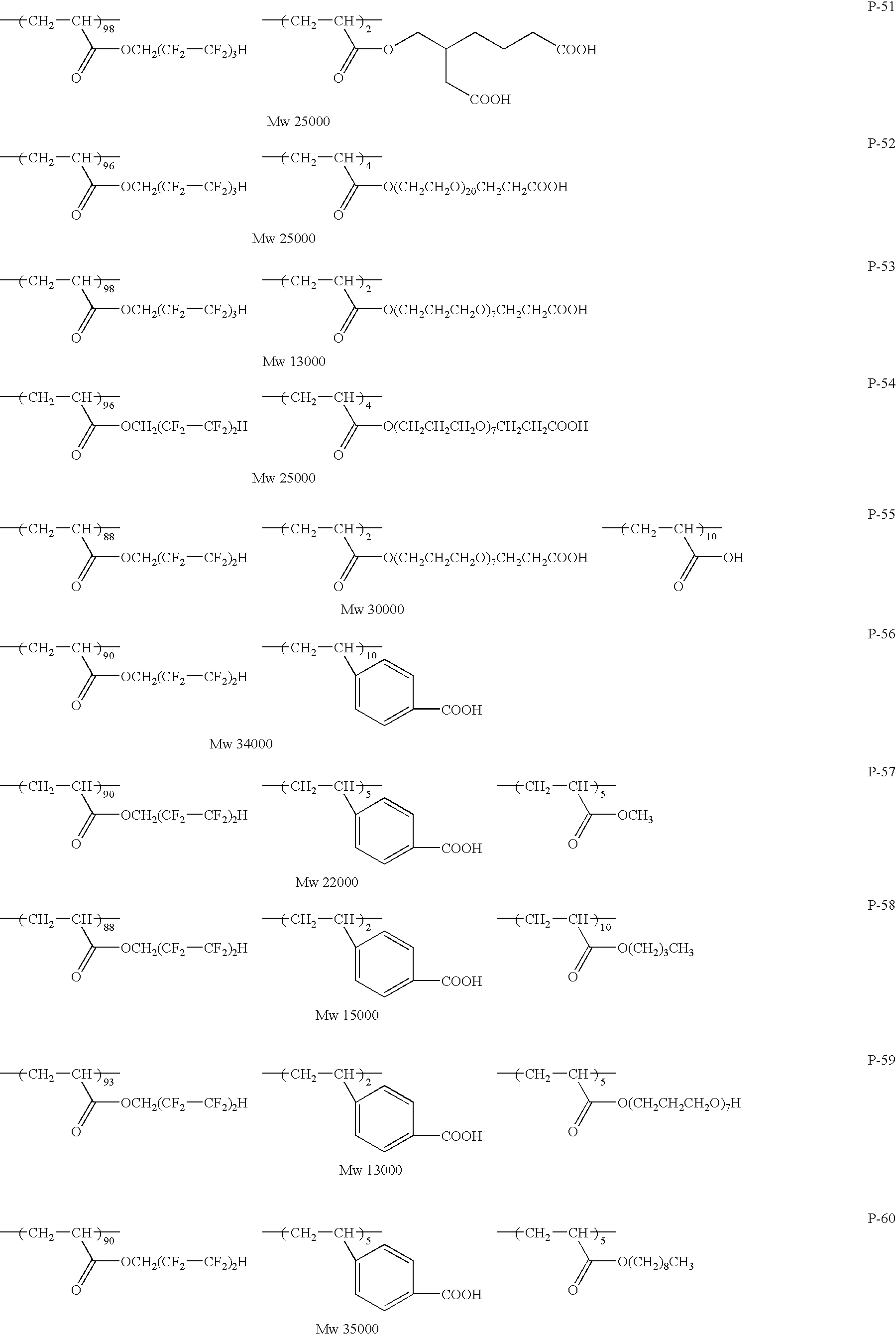

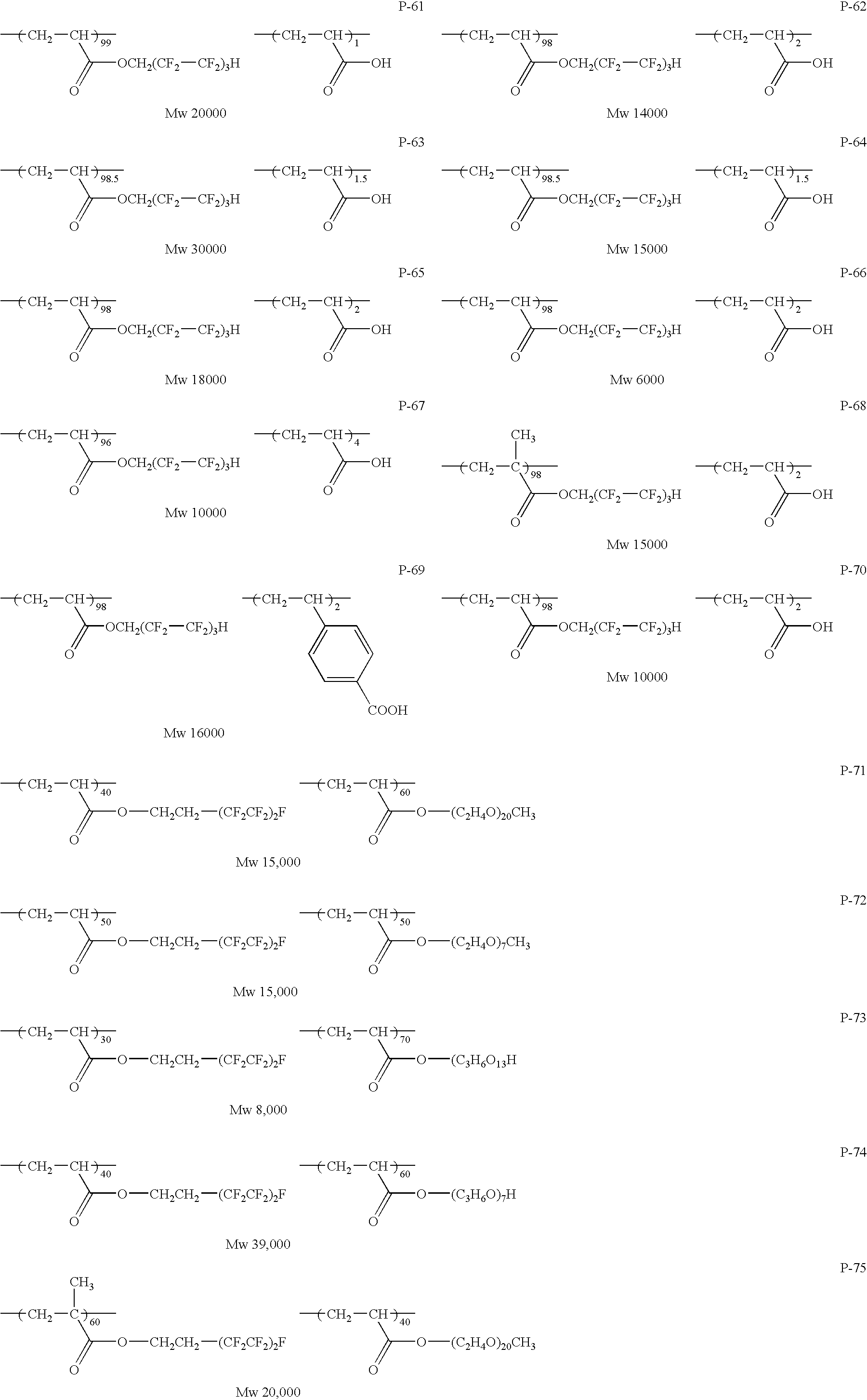

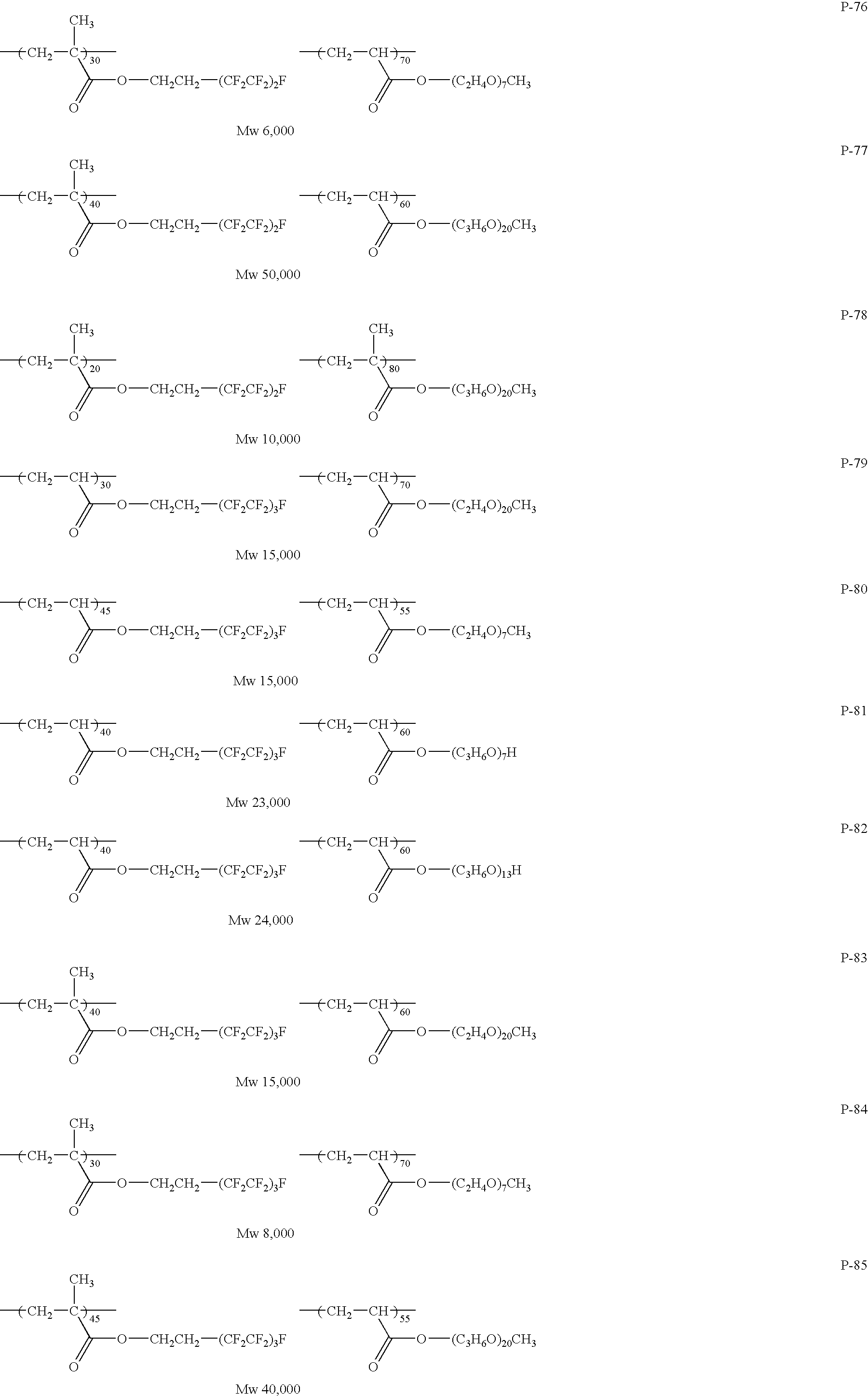

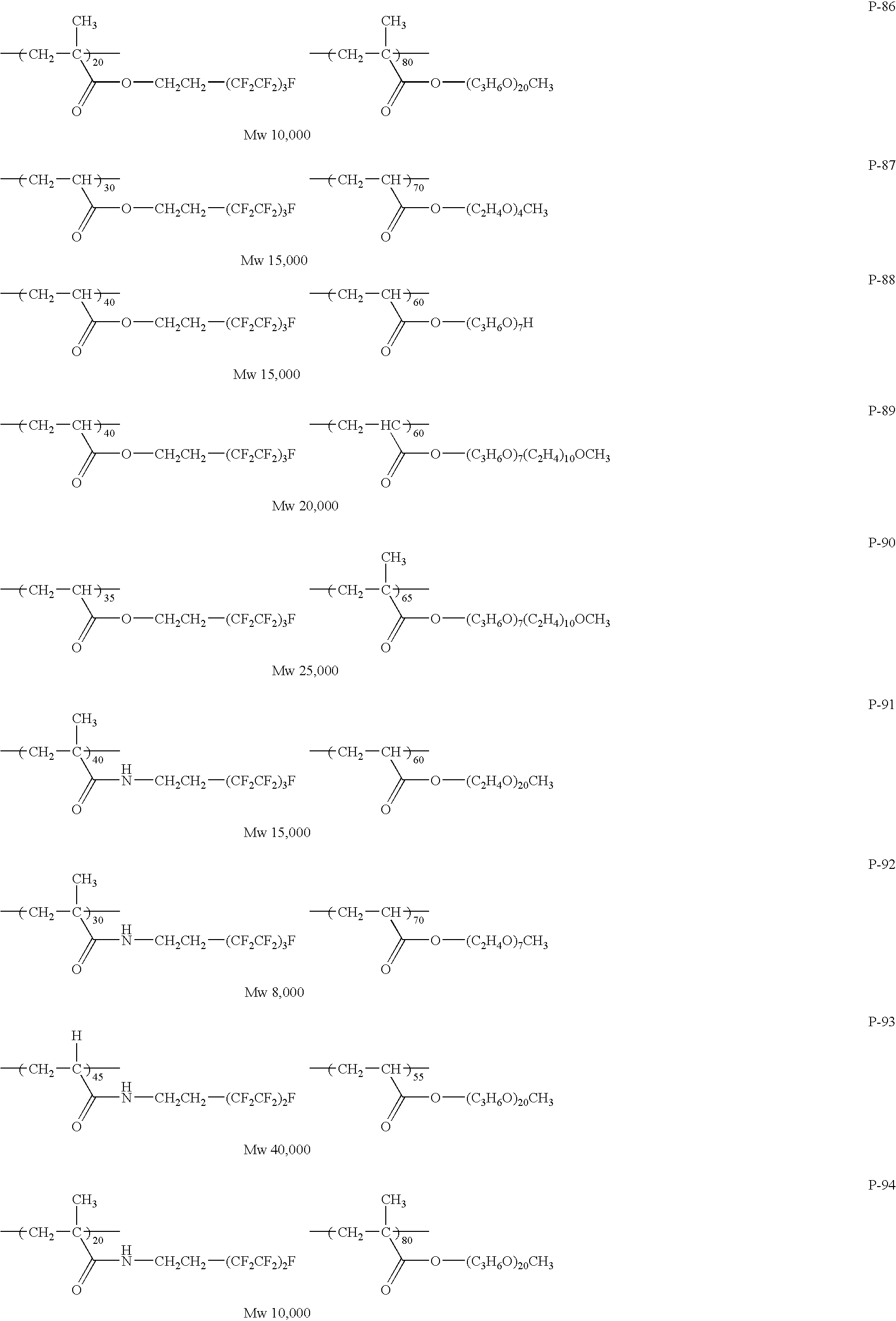

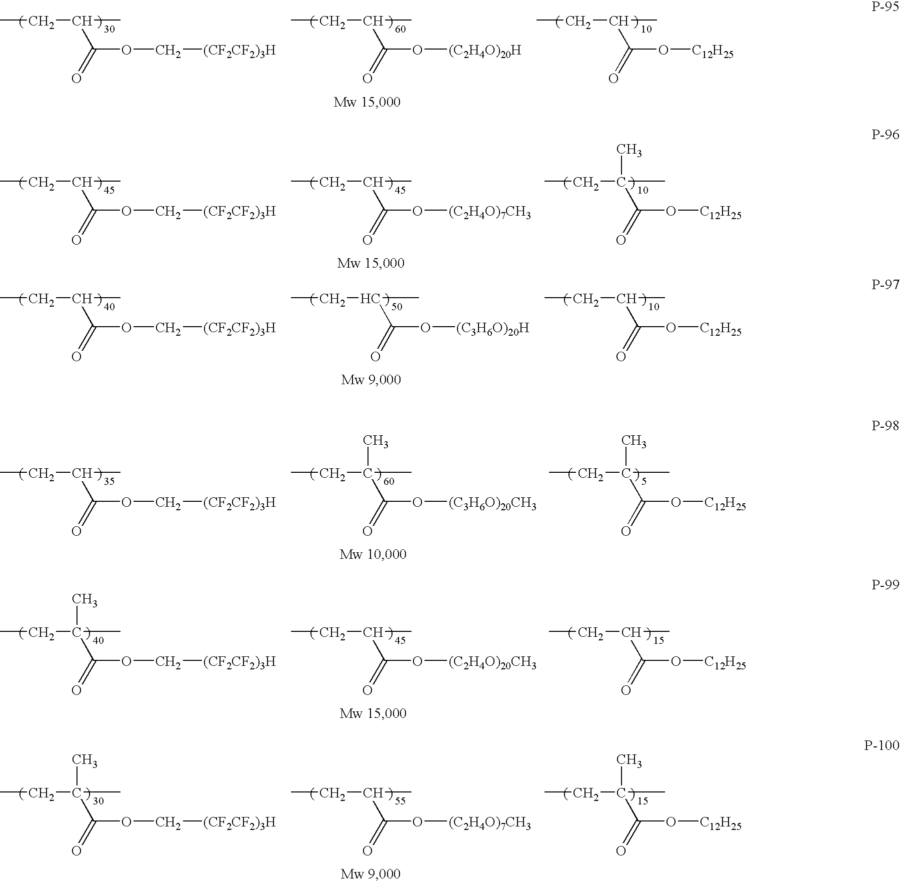

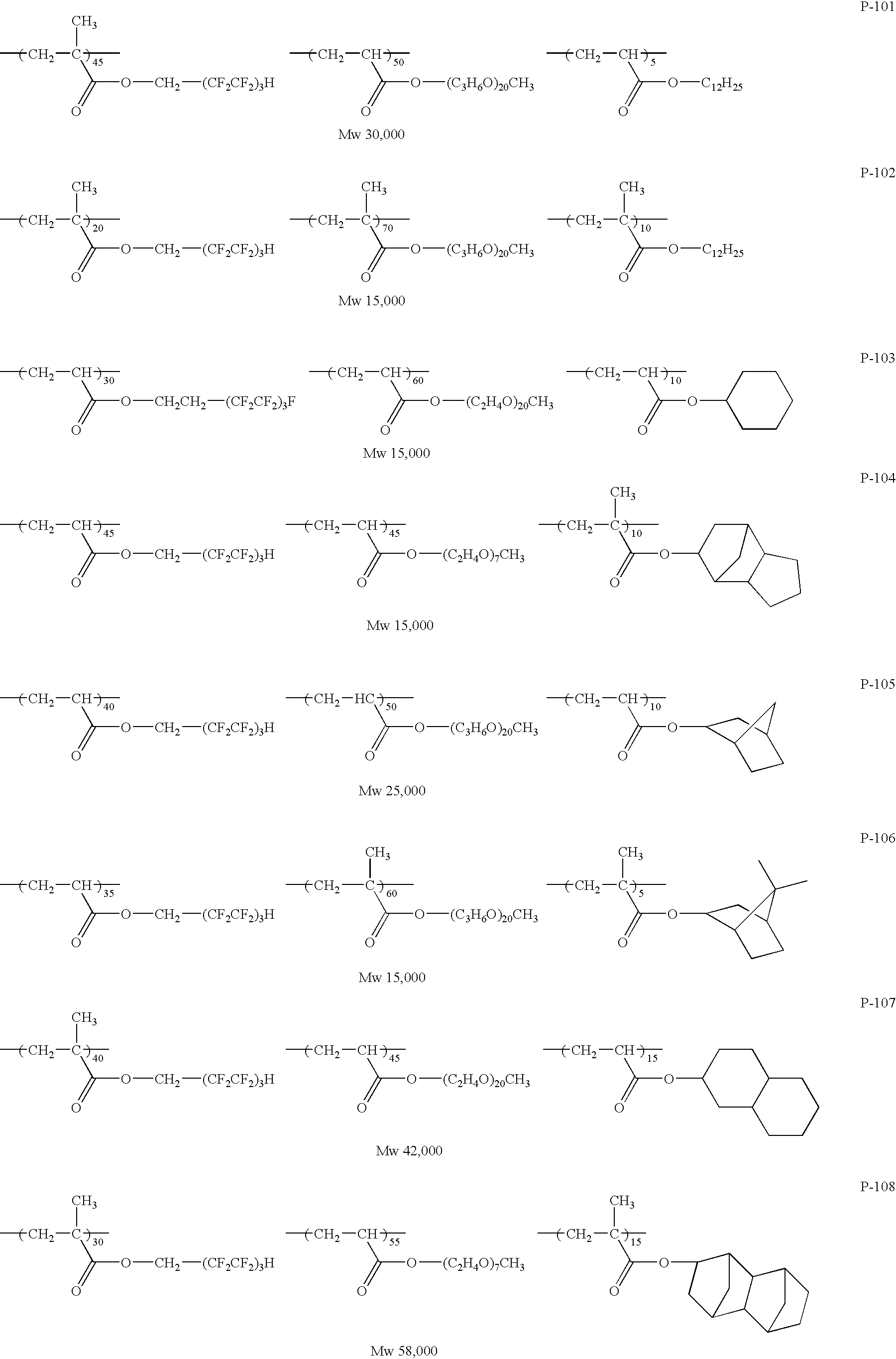

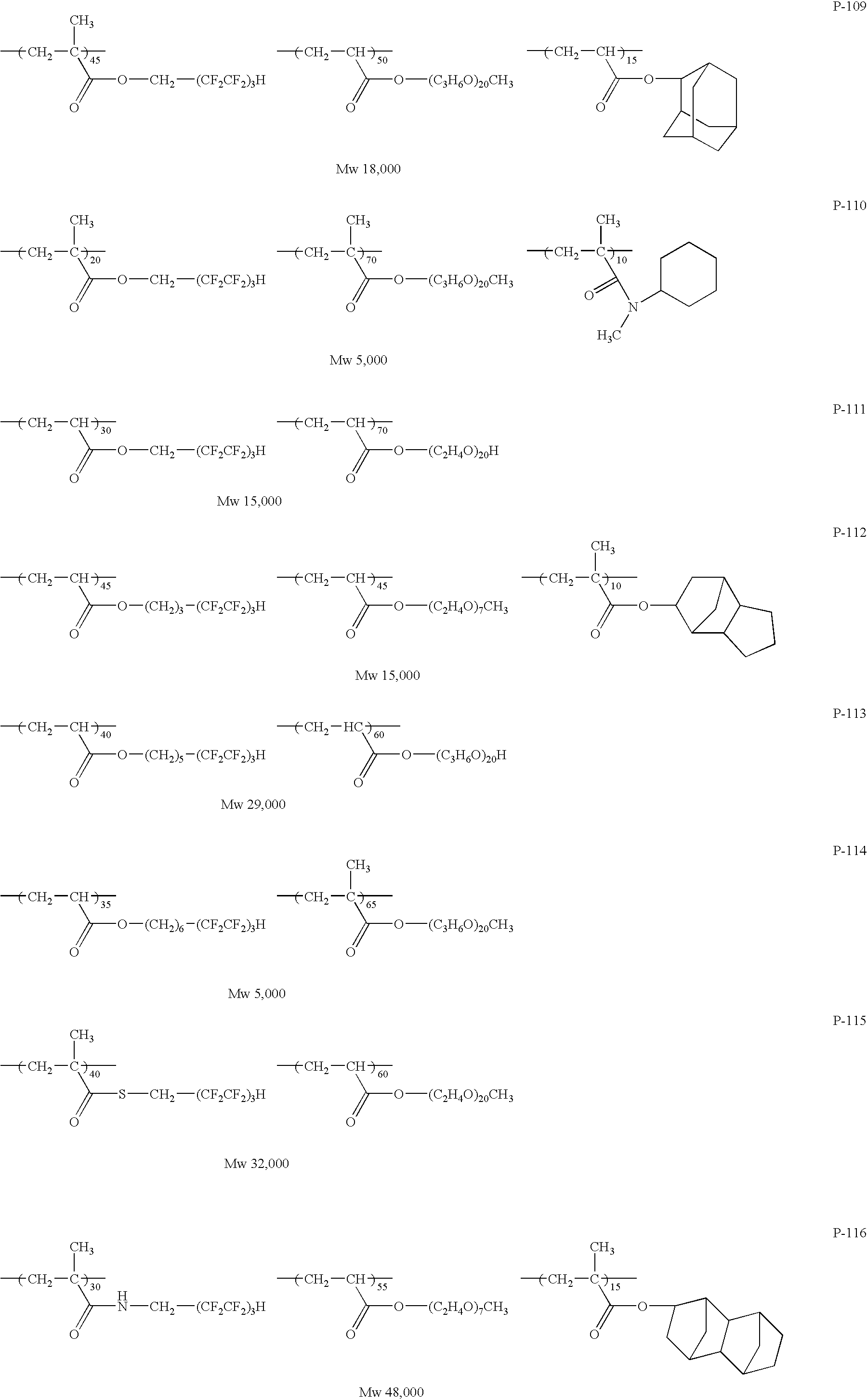

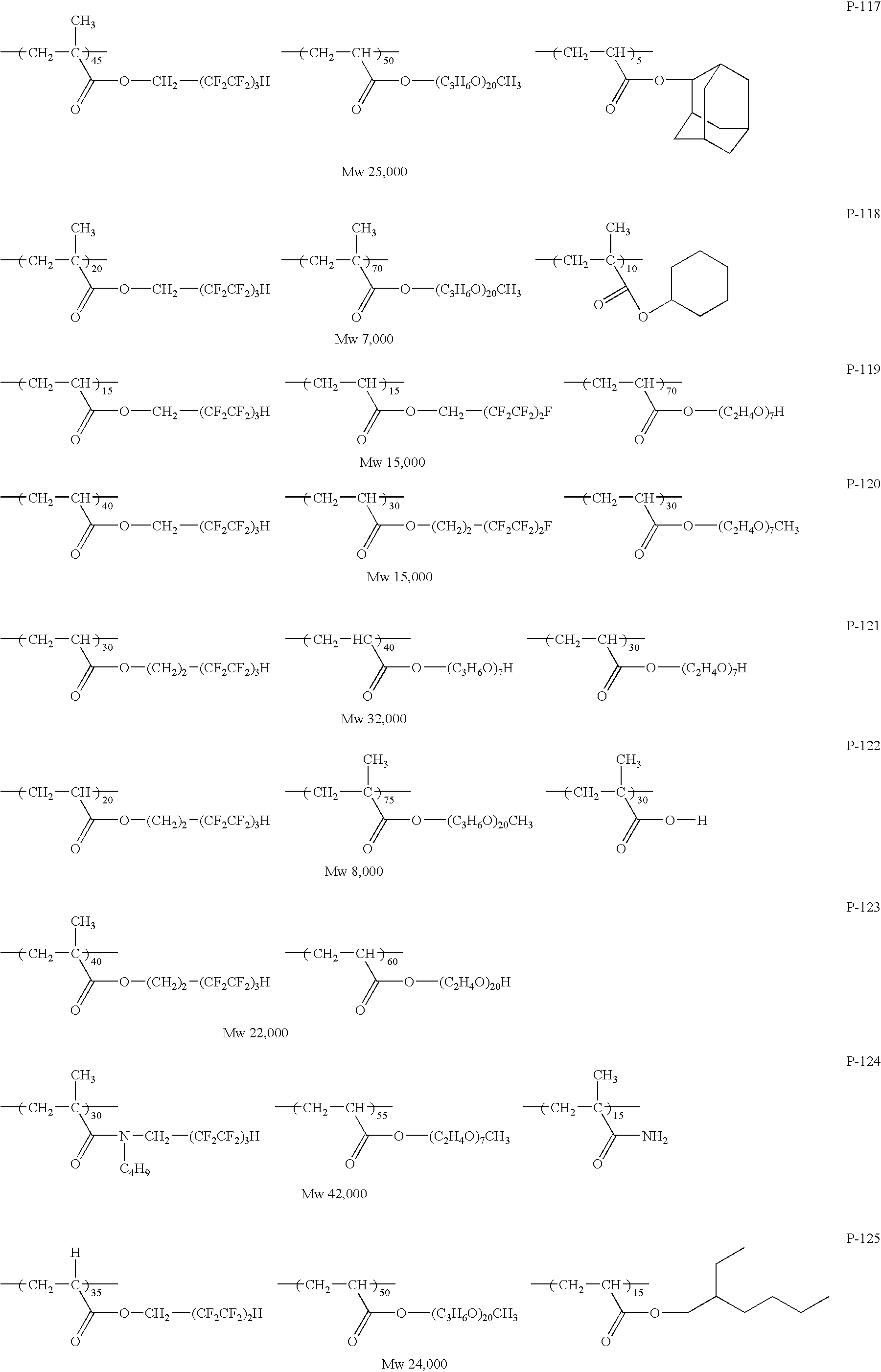

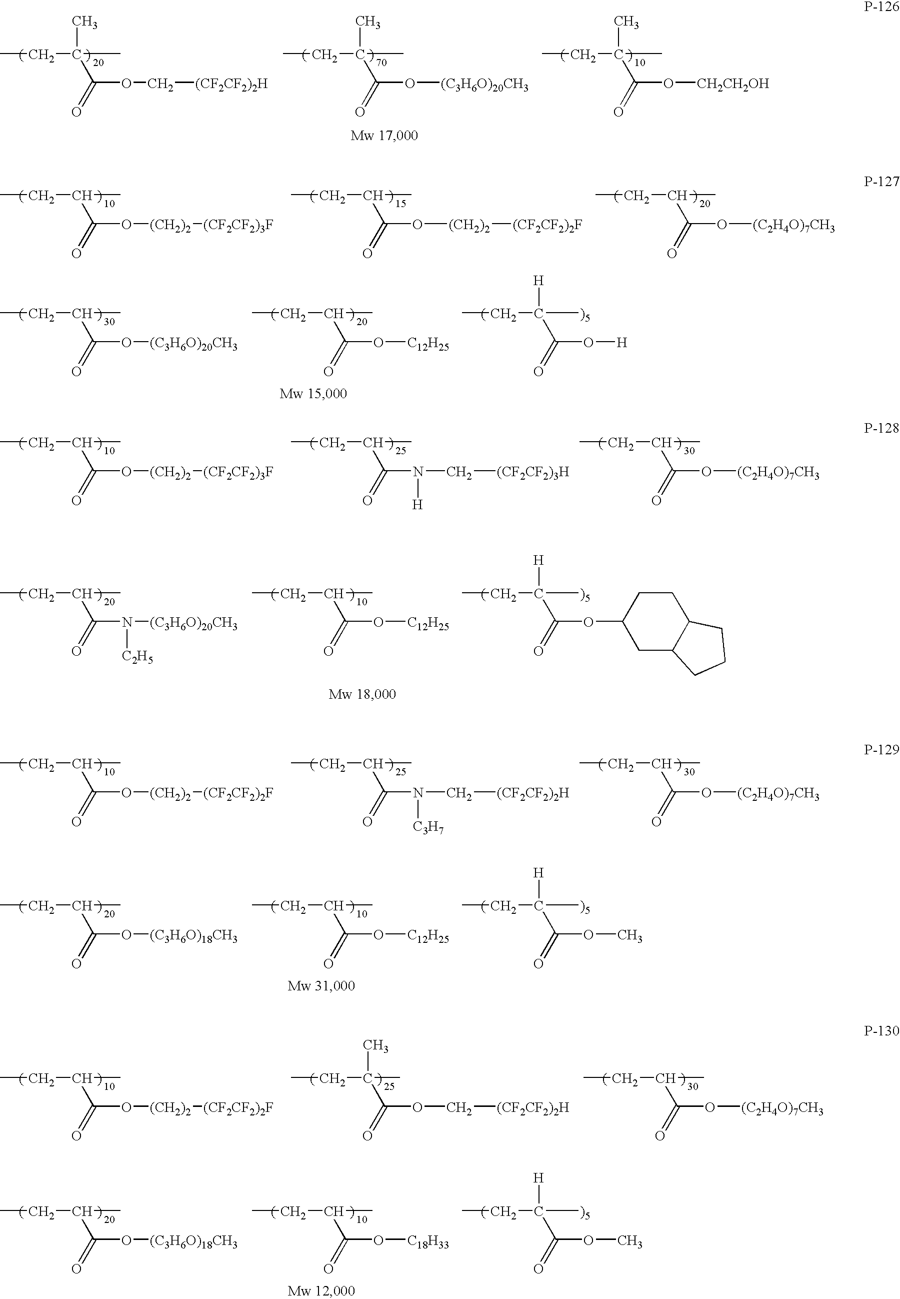

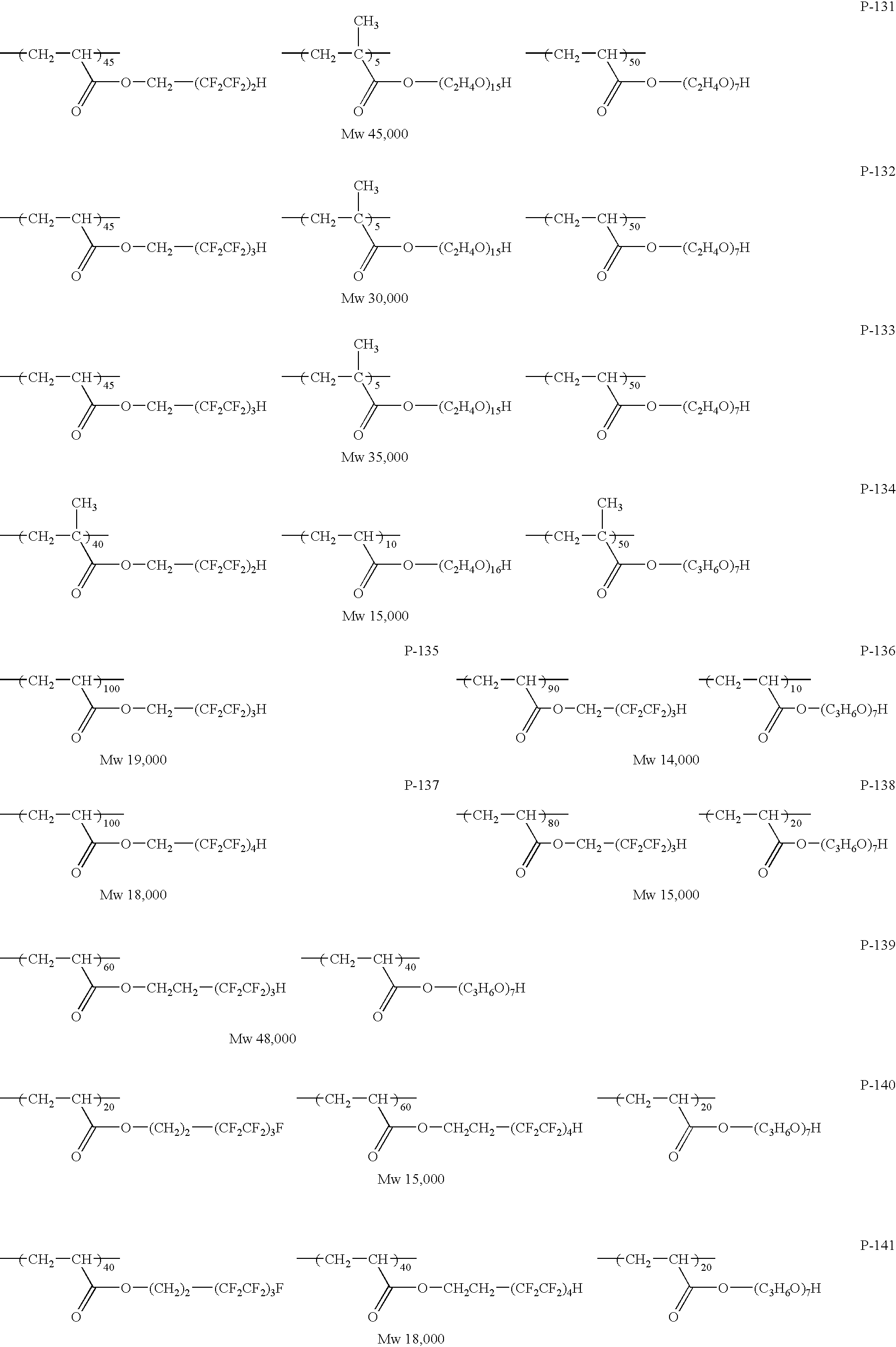

The fluorine-based polymer to be used in the invention can be produced by any known related art method. For example, there may be employed a method which comprises adding a general-purpose radical polymerization initiator to an organic solvent containing the aforementioned monomer having a fluoroaliphatic group, monomer having a hydrogen-bonding group or the like, and then subjecting the mixture to polymerization. Alternatively, in some cases, other addition-polymerizable unsaturated compounds may be added to the mixture which is then subjected to the same polymerization process as mentioned above. A dropping polymerization method which comprises polymerization with the dropping of monomers and initiator into the reaction vessel depending on the polymerizability of the various monomers can be effectively used to obtain a polymer having a uniform composition.

The content of the aforementioned fluorine-based polymer is preferably from 0.005 to 8% by mass, more preferably from 0.007 to 5% by mass based on the mass of the composition to be used in the formation of the optically anisotropic layer (excluding the solvent if a coating solution is prepared). When the content of the aforementioned fluorine-based polymer is 0.005% by mass or more, the effect of the invention can be sufficiently exerted. When the content of the aforementioned fluorine-based polymer is 8% by mass or less, there can occur no defects such as insufficient drying of coat layer and adverse effect on the properties of optically-compensatory sheet (e.g., uniformity in retardation). Thus, the fluorine-based polymer is preferably used in the above defined range.

In the invention, the aforementioned optically anisotropic layer preferably comprises two or more of the aforementioned fluorine-based polymers incorporated therein. All the two or more fluorine-based polymers preferably contain repeating units derived from the monomer represented by formula (1) and repeating units having a hydrophilic group represented by formula (2). The combination of these repeating units is not specifically limited, However, polymers comprising the same repeating unit derived from the monomer represented by formula (1) and different repeating units represented by formula (2) (e.g., different hydrophilic groups Q) may be combined.

In the invention, polymers having different monomer composition ratios (the kind of the monomers are the same, but their molar ratios are different) and polymers having different molecular weights (the kind and molar ratio of the monomers, but the molecular weight thereof are different) are considered “different polymers”.

### (Liquid Crystal Compound)

The aforementioned optically anisotropic layer is preferably designed such that the liquid crystal compound in the liquid crystal cell can be compensated while the liquid crystal display device is in black display state. The alignment of the liquid crystal compound in the liquid crystal cell during black display depends on the mode of the liquid crystal display device. For the details of the alignment of the liquid crystal compound in the liquid crystal cell, reference can be made to IDW'00, FMC7-2, pp. 411-414.

Examples of the liquid crystal compound to be used in the formation of the optically anisotropic layer include rod-shaped liquid crystal compounds and discotic liquid crystal compounds. These rod-shaped liquid crystal compounds and discotic liquid crystal compounds may be in the form of polymer liquid crystal or low molecular liquid crystal. Further, low molecular liquid crystals which have been crosslinked to exhibit liquid crystal properties no longer may be included.

### (Rod-Shaped Liquid Crystal Compound)

Examples of the rod-shaped liquid crystal compound employable herein include azomethines, azoxys, cyanobiphenyls, cyanophenylesters, benzoic acid esters, cyclohexanecarboxylic acid phenyl esters, cyanophenyl cyclophexanes, cyano-substituted phenylpyrimdines, alkoxy-substituted phenylpyrimidines, phenyldioxanes, tolans, and alkenyl cyclohexylbenzonitriles. Examples of the rod-shaped liquid crystal compound include metal complexes. A liquid crystal polymer containing a rod-shaped liquid crystal compound in its repeating unit, too, may be used. In other words, the rod-shaped liquid crystal compound may be connected to a (liquid crystal) polymer.

For the details of rod-shaped liquid crystal compound employable herein, reference can be made to “Kikan Kagaku Sosetsu (Quarterly Review of Chemistry Quarterly Review of Chemistry)”, vol. 22 (Chemistry of Liquid Crystal), 1994, Chapter 4, 7 and 11, The Chemical Society of Japan, and “Ekisho Debaisu Handobukku (Liquid Crystal Device Handbook)”, Chapter 3, The Japan Society for the Promotion of Science, The 142 Committee. The birefringence of the rod-shaped liquid crystal compound to be used in the invention preferably falls within the range of from 0.001 to 0.7.

The rod-shaped liquid crystal compound preferably has a polymerizable group to fix its alignment. The polymerizable group is preferably an unsaturated polymerizable group or epoxy group, more preferably unsaturated polymerizable group, most preferably ethylenically unsaturated polymerizable group.

### (Discotic Liquid Crystal Compound)

Examples of the discotic liquid crystal compound include benzene derivatives disclosed in C. Destrade et al's study report, “Mol. Cryst,”, vol. 71, page 111 (1981), truxene derivatives disclosed in C. Destrade and et al's study report, “Mol. Cryst.”, vol. 122, page 141 (1985), and “Physics lett”, A, vol. 78, page 82 (1990), cyclohexane derivatives disclosed in B. Kohne et al's study report, “Angew. Chem.”, vol. 96, page 70 (1984), and azacrown-based or phenyl acetylene-based macrocycles disclosed in J. M. Lehn et al's study report, “J. Chem. Commun.”, page 1,794 (1985), J. Zhang et al's study report, “J. Am. Chem. Soc.”, vol, 116, page 2,655 (1994).

Examples of the aforementioned discotic liquid crystal compounds include liquid crystal compounds comprising a nucleus disposed at the center of the molecule and straight-chain alkyl groups, alkoxy groups or substituted benzoyloxy groups disposed radially on the nucleus as side chains of the nucleus. The discotic liquid crystal compound is preferably a compound the molecules or molecule aggregate of which have a rotary symmetry and thus can be aligned as desired, In the case of the optically anisotropic layer formed by a discotic liquid crystal compound, the compound which is finally incorporated in the optically anisotropic layer no longer needs to be a discotic liquid crystal compound. For example, the low molecular discotic liquid crystal compound may have a group which reacts when heated or irradiated with light so that when heated or irradiated with light, the resulting reaction of the group causes the low molecular discotic liquid crystal compound to undergo polymerization or crosslinking to form a compound which has no longer liquid crystal properties. For the preferred examples of the discotic liquid crystal compounds, reference can be made to JP-A-8-50206. For the details of polymerization of discotic liquid crystal compound, reference can be made to JP-A-8-27284.

In order to fix the discotic liquid crystal compound by polymerization, it is necessary that polymerizable groups be connected to the disc-shaped core of the discotic liquid crystal compound as substituents, However, when polymerizable groups are directly connected to the disc-shaped core, it is difficult to keep the discotic liquid crystal molecules aligned as desired in the polymerization reaction. It is therefore preferred that connecting groups be incorporated in between the disc-shaped core and the polymerizable groups, Accordingly, the discotic liquid crystal compound having a polymerizable group is preferably a compound represented by formula (V).

D(-MP)ₙ (V)

In formula (V), D represents a discotic core; M represents a divalent connecting group; P represents a polymerizable group; and n represents an integer of from 4 to 12.

Examples of the discotic core (D) will be given below. In the following examples, MP (or PM) represents a combination of a divalent connecting group (M) and a polymerizable group (P).

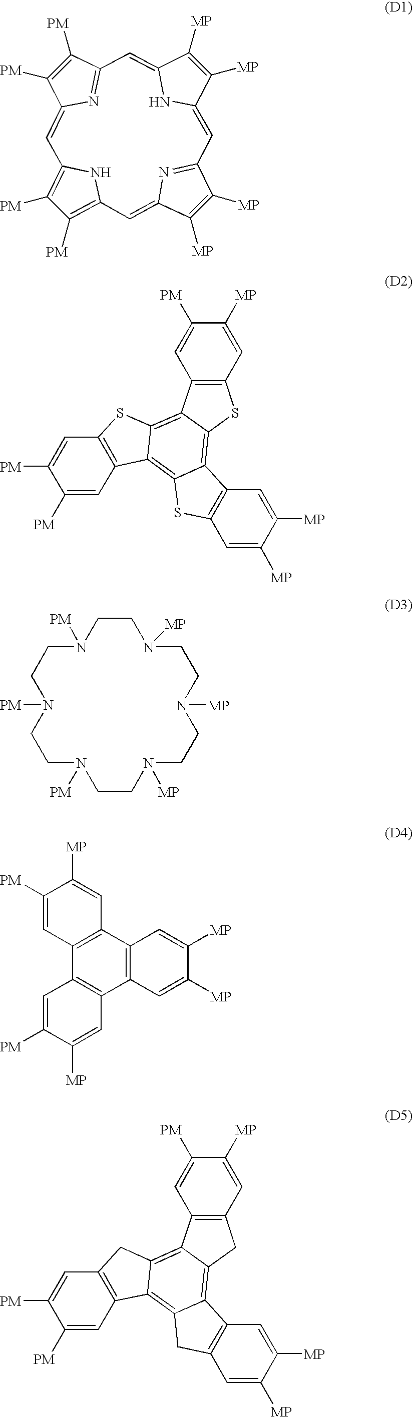

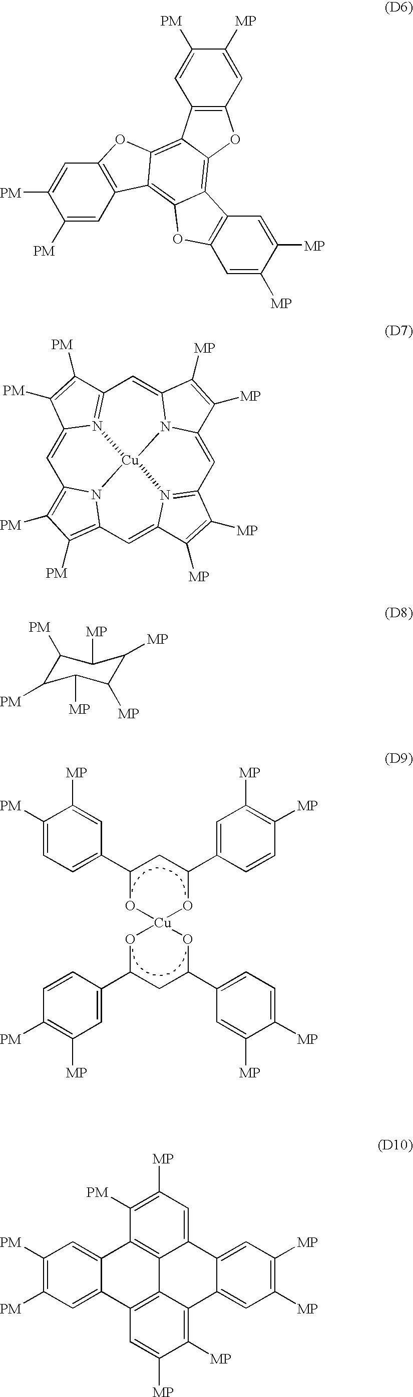

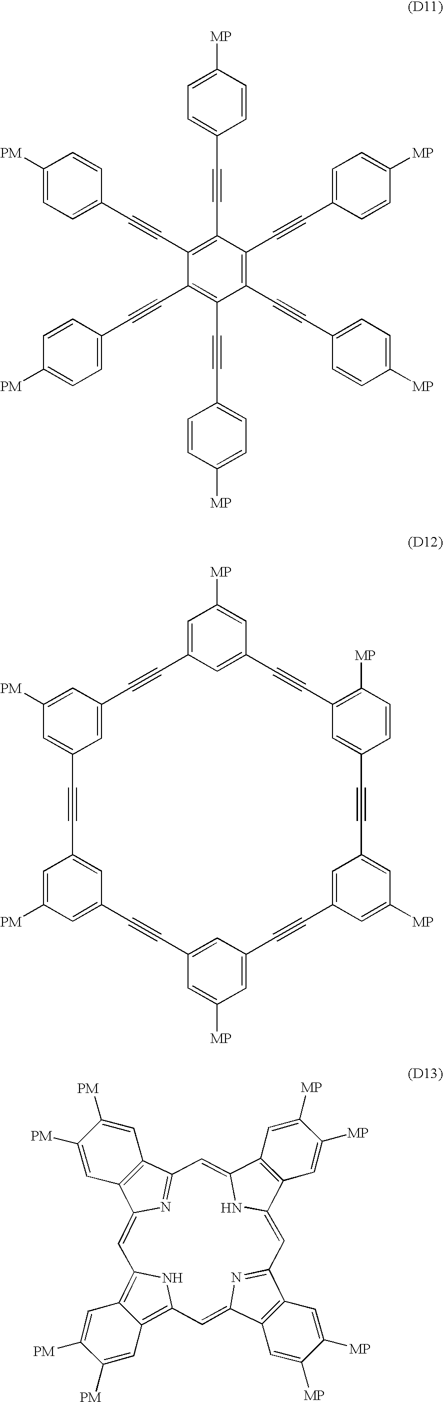

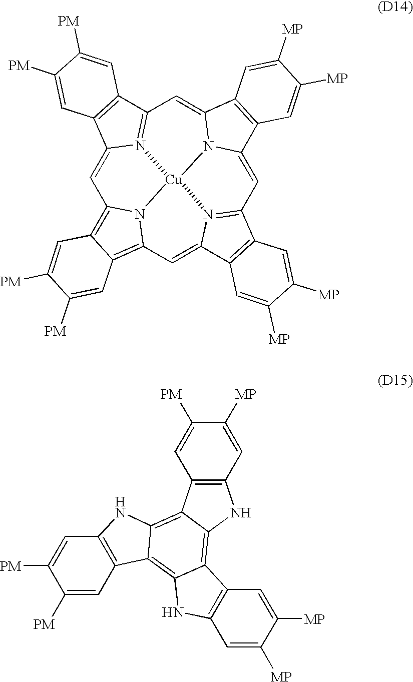

In formula (V), the divalent connecting group (M) is preferably a divalent connecting group selected from the group consisting of alkylene group, alkenylene group, arylene group, —CO—, —NH—, —O—, —S— and combination thereof. More preferably, the divalent connecting group (M) is a group formed by combining at least two divalent groups selected from the group consisting of alkylene group, alkenylene group, arylene group, —CO—, —NH—, —O— and —S—. Most preferably, the divalent connecting group (M) is a group formed by combining at least two divalent groups selected from the group consisting of alkylene group, alkenylene group, arylene group, —CO— and —O—. The number of carbon atoms in the alkylene group is preferably from 1 to 12. The number of carbon atoms in the alkenylene group is preferably from 2 to 12. The number of carbon atoms in the arylene group is preferably from 6 to 10. The alkylene group, alkenylene group and arylene group may have substituents (e.g., alkyl group, halogen atom, cyano, alkoxy group, acyloxy group).

Examples of the divalent connecting group (M) will be given below. The left moiety is connected to the discotic core (D) and the right moiety is connected to the polymerizable group (P)) AL represents an alkylene group or alkenylene group. AR represents an arylene group,

### M1: -AL-CO—O-AL-

### M2: -AL-CO—O-AL-O—

### M3: -AL-CO—O-AL-O-AL-

### M4: -AL-CO—O-AL-O—CO—

### M5: —CO-AR-O-AL-

### M6: —CO-AR-O-AL-O—

### M7: —CO-AR-O-AL-O—CO—

### M8: —CO—NH-AL-

### M9: —NH-AL-O—

### M10: —NH-AL-O—CO—

### M11: —O-AL-

### M12: —O-AL-O—

### M13: —O-AL-O—CO—

### M14: —O-AL, —O—CO—NH-AL-

### M15: —O-AL-S-AL-

### M16: —O—CO-AL-AR-O-AL-O—CO—

### M17: —O—CO-AR-O-AL-CO—

### M18: —O—CO-AR-O-AL-O—CO—

### M19: —O—CO-AR-O-AL-O-AL-O—CO—

### M20: —O—CO-AR-O-AL-O-AL-O-AL-O—CO—

### M21: —S-AL-

### M22: —S-AL-O—

### M23: —S-AL-O—CO—

### M24: —S-AL-S-AL-

### M25: —S-AR-AL-

The polymerizable group (P) in formula (V) is decided depending on the kind of the polymerization reaction to be effected. Examples of the polymerizable group (P) will be given below.

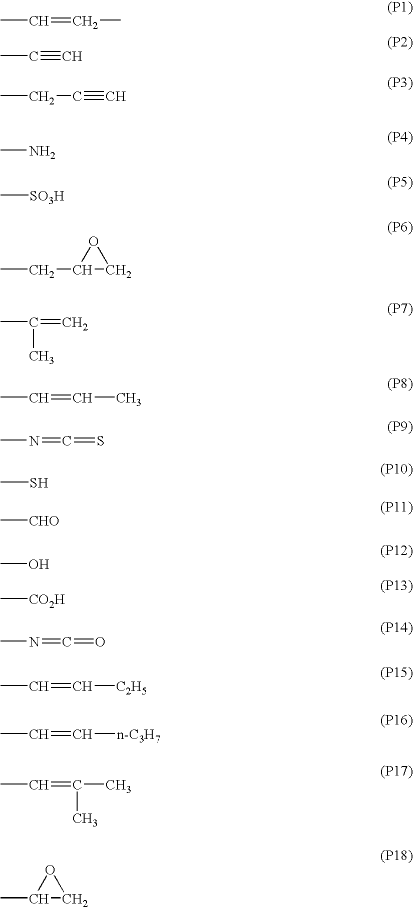

The polymerizable group (P) is preferably an unsaturated polymerizable group (P1, P2, P3, P7, P8, P15, P16, P17) or an epoxy group (P6, P18), more preferably an unsaturated polymerizable group, most preferably an ethylenically unsaturated polymerizable group (P1, P7, P8, P15, P16, P17). In formula (V), n represents an integer of from 4 to 12. In some detail, the integer is decided depending on the kind of the discotic core (D) The plurality of combinations of M and P may differ but are preferably the same. Two or more discotic liquid crystal compounds (e.g., molecules having asymmetric carbon atoms in the divalent connecting group and molecules free of these asymmetric carbon atoms) may be used in combination.

The discotic nematic liquid crystal phase-solid phase transition temperature of the discotic liquid crystal compound is preferably from 70° C. to 300° C., more preferably from 70° C. to 170° C.

In hybrid alignment, the angle of the major axis (disc surface) of the discotic compound with respect to the surface of the support, i.e., angle of tilt increases or decreases in the thickness direction of the optically anisotropic layer with the rise of the distance from the surface of the polarizing film. The angle of tilt preferably increases with the rise of the distance from the surface of the polarizing film. The change of tilt angle may be a continuous increase, a continuous decrease, an intermittent increase, an intermittent decrease, a change involving continuous increase and continuous decrease or an intermittent change involving increase and decrease. The intermittent change involves a region in which the tilt angle shows no change in the course of thickness direction. Even when such a region in which the tilt angle shows no change is involved, it suffices if the tilt angle increases or decreases as a whole. However, the tilt angle preferably shows continuous change.

In the optically anisotropic layer having discotic compounds or rod-shaped compounds aligned, it is difficult to directly and accurately measure the tilt angle (angle between the physical symmetrical axis in a discotic compound or rod-shaped compound and the interface of an optically anisotropic layer) θ1 on one surface of the optically anisotropic layer and the tilt angle θ2 on the other side thereof. In the present specification, θ1 and θ2 are calculated by the following method. This method doesn't express accurately the actual alignment of the invention but is a method effective for the expression of the relative relationship of some of optical properties of optical films.

In this method, in order to facilitate calculation, the following two points are assumed to determine the tilt angle at two interfaces of optically anisotropic layer.

1. It is assumed that the optically anisotropic layer be a multi-layered material composed of a layer containing a discotic compound or rod-shaped compound. It is further assumed that the minimum unit layer constituting the multi-layered material (It is assumed that the tilt angle of the discotic compound or rod-shaped compound be uniform in the layer) be optically monoaxial.

2. It is assumed that the tilt angle of the various layers show a linear monotonous change along the thickness direction of the optically anisotropic layer,

The calculation method will be described in detail below.

(1) In a plane where the tilt angle of the various layers show a linear monotonous change along the thickness direction of the optically anisotropic layer, the retardation value is measured at three or more angles with the angle of incidence of measuring light on the optically anisotropic layer varied. In order to simplify measurement and calculation, the retardation value is preferably measured at three angles, i.e., −40°, 0° and +40°, provided that the normal line direction to the optically anisotropic layer is defined as 0°. For this measurement, KOBRA-21ADH and KOBRA-WR (produced by Ouji Scientific Instruments Co., Ltd.), a Type AEP-100 transmission type ellipsometer (produced by Shimadzu Corporation), M150 and M520 (produced by JASCO Corporation), and ABR10A (produced by The Optronics Co., Ltd.) can be used.

(2) In the aforementioned model, the refractive index of the various layers with respect to normal light and abnormal light are assumed to be no and ne (ne is the same in all these layers. This can apply also to no), respectively, and the thickness of the entire multi-layered material is assumed to be d. On the assumption that the direction of tilt in the various layers and the direction of one optical axis of the various layers coincide with each other, fitting is made with the tilt angle θ1 on one side of the optically anisotropic layer and the tilt angle θ2 on the other side of the optically anisotropic layer as variable such that the calculated value of the angle dependence of the retardation value of the optically anisotropic layer coincides with the measured value thereof. Thus, θ1 and θ2 are calculated.

Here, known values such as the literature value and the catalogue value can be used as no and ne. When the value is unknown, it can be measured by means of an Abbe refractometer. The thickness of the optically anisotropic layer can be measured by means of an optical interferometer, a scanning electron microscope and the like.

The average direction of the major axis (disc surface of molecules in the case of discotic liquid crystal compound) of the liquid crystal compound (average of major axis of the various molecules) can be normally adjusted by properly selecting the material of the liquid crystal compound or alignment film or the rubbing method. Further, the direction of the major axis of the liquid crystal compound on the surface side (air side) can be normally adjusted by properly selecting the kind of the additives to be used with the liquid crystal compound.

Examples of the additives to be used with the liquid crystal compound include plasticizers, surface active agents, and polymerizable monomers and polymers. The degree of change of the direction of alignment of the major axis can be adjusted by properly selecting the liquid crystal compound and the additives as in the aforementioned case.

The plasticizer, surface active agent and polymerizable monomer to be used with the liquid crystal compound preferably have compatibility with the liquid crystal compound to cause the change of the tilt angle of the liquid crystal compound. Alternatively, these additives preferably never inhibit the alignment of the liquid crystal compound. Preferred among these additive components are polymerizable monomers (e.g., compounds having vinyl group, vinyloxy group, acryloyl group or methacryloyl group). The amount of the aforementioned compounds to be added is normally from 1% to 50% by mass, preferably from 5% to 30% by mass based on the amount of the liquid crystal compound. In order to attain a high alignment order parameter, it is preferred that the aforementioned compounds be used in a small amount. When monomers having four or more polymerizable reactive functional groups are used in admixture, the adhesion between the alignment film and the optically anisotropic layer can be enhanced.

The optically anisotropic layer may comprise the aforementioned fluoroaliphatic group-containing polymer incorporated therein. Other polymers may be used with the liquid crystal compound. These polymers preferably have some compatibility with the liquid crystal compound to cause the change of tilt angle of the liquid crystal compound.

Examples of the polymers employable herein include cellulose esters. Preferred examples of the cellulose esters include cellulose acetate, cellulose acetate propionate, hydroxypropyl cellulose, and cellulose acetate butyrate. However, the effect of the invention can be exerted remarkably when the aforementioned fluoroaliphatic group-containing polymer which can effectively accelerate the hybrid alignment of the liquid crystal compound is used and the amount of the related art hybrid alignment accelerator such as the aforementioned cellulose ester causing the deterioration of alignment order parameter or haze is reduced. The amount of the aforementioned polymers to be added is properly adjusted in view of the amount of the fluoroaliphatic group-containing polymer. From the standpoint of enhancement of alignment order parameter, the amount of these polymers to be used is preferably minimized.

A composition containing at least one liquid crystal compound, the aforementioned fluorine-based polymer and optionally various compounds is prepared as a coating solution. The coating solution thus prepared is spread over the surface of the alignment film or the like. The liquid crystal compound molecules are then aligned to form an optically anisotropic layer. The aforementioned optically anisotropic layer exhibits an optical anisotropy developed by the alignment of the liquid crystal compound molecules.

As the solvent to be used in the preparation of the coating solution there is preferably used an organic solvent. Examples of the organic solvent employable herein include amides (e.g., N,N-dimethylformamide), sulfoxides (e.g., dimethyl sulfoxide), heterocyclic compounds (e.g., pyridine), hydrocarbons (e.g., benzene, hexane), alkyl halides (e.g., chloroform, dichloromethane, tetrachloroethane), esters (e.g., methyl acetate, butyl acetate), ketones (e.g., acetone, methyl ethyl ketone), and ethers (e.g., tetrahydrofurane, 1,2-diemthoxyethane). Preferred among these organic solvents are alkyl halides and ketones. Two or more of these organic solvents may be used in combination.

The spreading of the coating solution can be carried out by any known method (e.g., wire bar coating method, extrusion coating method, direct gravure coating method, reverse gravure coating method, die coating method)

In order to prepare an optical film having an extremely high uniformity as in the Invention, the surface tension of the coating solution is preferably 25 mN/m or less, more preferably 22 mN/m or less.

### (Fixing of Alignment of Liquid Crystal Compound Molecules)

The aforementioned coating solution is spread over the surface of the alignment film or the like, The molecules of liquid crystal compound are then aligned. The liquid crystal molecules thus aligned can be fixed aligned, The fixing of alignment is preferably carried out by polymerization reaction. Examples of the polymerization reaction include heat polymerization reaction involving the use of a heat polymerization initiator and photopolymerization reaction involving the use of a photopolymerization initiator The photopolymerization reaction is preferred.

Examples of the photopolymerization initiator include α-carbonyl compounds (disclosed in U.S. Pat. Nos. 2,367,661 and 2,367,670), acyloin ethers (disclosed in U.S. Pat. No. 2,448,828), a-hydrocarbon-substituted aromatic acyloin compounds (disclosed in U.S. Pat. No. 2,722,512), polynuclear quinone compounds (disclosed in U.S. Pat. Nos. 3,046,127 and 2,951,758), combinations of triaryl imidazole dimer and p-aminophenyl ketone (disclosed in U.S. Pat. No. 3,549,367), acridine and phenazine compounds (disclosed in JP-A-60-105667 and U.S. Pat. No. 4,239,850), and oxadiazole compounds (disclosed in U.S. Pat. No. 4,212,970).

The amount of the photopolymerization initiator to be used is preferably from 0.01% to 20% by mass, more preferably from 0.5% to 5% by mass based on the solid content of the coating solution.

As the light beam to be emitted for the polymerization of the liquid crystal molecules there is preferably used ultraviolet ray.

The radiation energy is preferably from 20 mJ/cm² to 50 J/cm², more preferably from 20 mJ/cm² to 5,000 mJ/cm², even more preferably from 100 mJ/cm² to 800 mJ/cm². In order to accelerate the photopolymerization reaction, radiation may be effected under heating. However, in order to enhance the alignment order parameter, irradiation is preferably effected at a temperature close to ordinary temperature rather than upon heating. A protective layer may be provided on the optically anisotropic layer.

### (Alignment Film)

The formation of the aforementioned optically anisotropic layer is preferably accomplished by the use of an alignment film. Preferred examples of the alignment film to be used in the formation of the aforementioned optically anisotropic layer include those made of crosslinked polymers, more preferably crosslinked polyvinyl alcohol, described in JP-A-8-338913. The alignment film can be formed by any related art known coating method (e.g., wire bar coating method, extrusion coating method, direct gravure coating method, reverse gravure coating method, die coating method).

The alignment film preferably has a thickness of 10 μm or less.

### (Support)

The optically-compensatory sheet of the invention may have a support for supporting the aforementioned optically anisotropic layer. The support is preferably glass or a transparent polymer film. The support is preferably transparent. In some detail, the support of the invention preferably has a light transmittance of 80% or more. Examples of the polymer constituting the polymer film include cellulose esters (e.g., cellulose triacetate, cellulose diacetate), norbornene-based polymers, and polymethyl methacrylates. Commercially available polymers (including Arton and Zeonex (trade name)) may be also used.

Preferred among these polymers are cellulose esters. Lower aliphatic acid esters of cellulose are more desirable. The term “lower aliphatic acid” as used herein is meant to indicate an aliphatic acid having 6 or less carbon atoms. The number of carbon atoms in the aliphatic acid is preferably 2 (cellulose acetate), 3 (cellulose propionate) or 4 (cellulose butyrate) Cellulose acetate is particularly preferred. A mixed aliphatic acid ester such as cellulose acetate propionate and cellulose acetate butyrate may be also used.

Even a polymer which can easily exhibit birefringence such as polycarbonate and polysulfone, that have heretofore been known, can be used as optically-compensatory sheet of the invention when controlled in development of birefringence by modifying its molecules as disclosed in WO00/26705.

In the case where the optically-compensatory sheet of the invention is used as protective film for polarizing plate or retardation film, as the polymer film to be used as a support for the optically anisotropic layer there is preferably used a cellulose acetate having a percent acetylation of from 55.0% to 62.5%, more preferably from 57.0% to 62.0%.

The term “percent acetylation” as used herein is meant to indicate the mass of acetic acid bonded per mass of cellulose unit The percent acetylation is determined by the measurement and calculation of acylation degree according to ASTM: D-817-91 (testing method on cellulose acylate, etc.).

The viscosity-average polymerization degree (DP) of the cellulose acetate is preferably 250 or more, more preferably 290 or more. The cellulose acetate preferably has a sharp molecular weight distribution Mw/Mn (in Mw represents weight-average molecular weight and Mn represents a number-average molecular weight) as determined by gel permeation chromatography. In some detail, Mw/Mn is preferably from 1.0 to 1.7, more preferably from 1.0 to 1.65, most preferably from 1.0 to 1.6.

A cellulose acylate tends to have hydroxyl group substituted less in the 6-position rather than uniformly in the 2-position, 3-position and 6-position. The cellulose acylate to be used in the invention preferably has a cellulose substitution degree in the 6-position which is the same as or greater that that in the 2- and 3-positions.

The proportion of the substitution degree in the 6-position in the sum of the substitution degree in the 2-, 3- and 6-positions is preferably from 30% to 40%, more preferably from 31% to 40%, most preferably from 32% to 40%. The substitution degree in the 6-position is preferably 0.88 or more.

The substitution degree in the various positions can be measured by NMR.

A cellulose acetate having a high substitution degree in the 6-position can be synthesized according to Synthesis Examples 1, 2 and 3 disclosed in JP-A-11-5851, paragraph [0043]-[0044], [0048]-[0049] and [0051]-[0052], respectively.

### (Ellipsoidal Polarizing Plate)

The invention also concerns an ellipsoidal comprising at least a polarizing film and an optically-compensatory sheet of the invention. For example, the optically-compensatory sheet of the invention may be laminated on a polarizing plate. Alternatively, the optically-compensatory sheet of the invention may be used as a protective film for protecting the polarizing film of the polarizing plate. The use of the configuration of ellipsoidal polarizing plate makes it possible to remarkably develop the function of the optically-compensatory sheet of the invention.

### (Polarizing Film)

The polarizing film of the invention which can be used in the invention (occasionally referred to as “polarizer”) is preferably a coated polarizing film such as those produced by Optiva Inc, or a polarizing film made of a binder and an iodine or dichroic dye.

Iodine and the dichroic dye in the polarizing film are aligned in the binder to exhibit its polarizing capacity. Iodine and the dichroic dye are preferably aligned along the binder molecules. Alternatively, the dichroic dye is preferably self-organized as in liquid crystal so that it is aligned in one direction.

The general-purpose polarizers can be prepared by dipping a stretched polymer in an iodine or dichroic dye solution in a bath so that the binder is impregnated with iodine or dichroic dye.

In the general-purpose polarizing film, iodine or dichroic dye is distributed in the polarizing film over a range from the surface of the polymer to a depth of about 4 μm (about 8 μm in total). In order to obtain sufficient polarizing properties, the general-purpose polarizing film needs to have a thickness of at least 10 μm. The degree of penetration can be controlled by the concentration of iodine or dichroic dye solution, the temperature of the iodine or dichroic dye bath and the dipping time.

As mentioned above, the lower limit of the thickness of the binder is preferably 10 μm. On the other hand, the upper limit of the thickness of the binder is not specifically limited but is preferably as small as possible from the standpoint of prevention of light leakage which would occur when the polarizing plate is used in liquid crystal display devices. The thickness of the binder is preferably not greater than the thickness of the present general-purpose polarizing plate (about 30 μm), more preferably 25 μm or less, even more preferably 20 μm or less. When the thickness of the binder is 20 μm or less, light leakage can be difficultly observed with 17-inch liquid crystal display devices.

The binder for the polarizing film may be crosslinked. As the crosslinked binder there may be used a polymer which can be crosslinked itself. Polymers or binders obtained by introducing a functional group into the polymers can be reacted with each other by light, heat or pH change to form a polarizing film.

The polymer may be provided with a crosslinked structure in the presence of a crosslinking agent. The introduction of the crosslinked structure can be carried out by introducing a connecting group derived from a crosslinking agent which is a highly active compound into the gap between the binder molecules in the presence of the crosslinking agent.

Crosslinking is normally carried out by spreading a coating solution containing a polymer or a mixture of a polymer and a crosslinking agent over a transparent support, and then heating the coated material. It suffices if the desired durability can be assured at the stage of final product. Therefore, crosslinking may be effected at any step until the step of obtaining the final polarizing plate.

As the binder for polarizing film there may be used any of polymers which can be crosslinked themselves and polymers which can be crosslinked by a crosslinking agent. Examples of these polymers include polymethyl methacrylates, polyacrylic acids, polymethacrylic acids, polystyrenes, gelatins, polyvinyl alcohols, modified polyvinyl alcohols, poly(N-methylolacrylamide), polyvinyltoluenes, chlorosulfonated polyethylenes, nitrocelluloses, chlorinated polyolefins (e.g., polyvinyl chloride), polyesters, polyimides, polyvinyl acetates, polyethylenes, carboxymethyl celluloses, polypropylenes, and polycarbonates and copolymers thereof (e.g., acrylic acid-methacrylic acid copolymer, styrene-maleinimide copolymer, styrene-vinyltoluene copolymer, vinyl acetate-vinyl chloride copolymer, ethylene-vinyl acetate copolymer). Water-soluble polymers (e.g., poly(N-methylolacrylamide), carboxymethyl cellulose, gelatin, polyvinyl alcohol, modified polyvinyl alcohol) are preferably used. Gelatins, polyvinyl alcohols and modified polyvinyl alcohols are more preferably used. Polyvinyl alcohols and modified polyvinyl alcohols are most preferably used.

The percent saponification of the polyvinyl alcohol and modified polyvinyl alcohol is preferably from 70% to 100%, more preferably from 80% to 100%, most preferably from 95% to 100%. The polymerization degree of the polyvinyl alcohol is preferably from 100 to 5,000.

The modified polyvinyl alcohol is obtained by subjecting a polyvinyl alcohol to copolymerization modification, chain transfer modification or block polymerization modification so that a modifying group is introduced thereinto. In the copolymerization modification process, as modifying groups there can be introduced various groups such as —COONa, —Si(OH)₃, N(CH₃)₃Cl, C₉H₁₉COO—, —SO₃Na and —C₁₂H₂₅. In the chain transfer modification, as modifying groups there can be introduced various groups such as —COONa, —SH, and —SC₁₂H₂₅. The polymerization degree of the modified polyvinyl alcohol is preferably from 100 to 3,000. For the details of modified polyvinyl alcohols, reference can be made to JP-A-8-338913, JP-A-9-152509 and JP-A-9-316127.

Unmodified polyvinyl alcohols and alkylthio-modified polyvinyl alcohols having a percent saponification of from 85% to 95% are particularly preferably preferred.

Two or more of polyvinyl alcohols and modified polyvinyl alcohols may be used in combination.

When the binder crosslinking agent is added in a large amount, the moist heat resistance of the polarizing film can be enhanced. However, when the crosslinking agent is added in an amount of 50% by mass or more based on the amount of the binder, the alignability of iodine or dichroic dye is lowered. The amount of the crosslinking agent to be added is preferably from 0.1% to 20% by mass, more preferably from 1.2% to 1.45% by mass based on the amount of the binder.

The binder contains unreacted crosslinking agent in some amount even after the termination of the crosslinking reaction. However, the amount of the crosslinking agent left unreacted is preferably 1.0% by mass or less, more preferably 0.5% by mass or less based on the amount of the binder When the binder layer contains a crosslinking agent in an amount of more than 1.0% by mass, the resulting polarizing film may leave something to be desired in durability. In other words, when a liquid crystal display device having a polarizing film having a great residual crosslinking agent content incorporated therein is used over an extended period of time or allowed to stand in a high temperature and humidity atmosphere, the polarization can be deteriorated.

For the details of the crosslinking agent, reference can be made to U.S. Reissued Pat. No. 23,297. Boron compounds (e.g., boric acid, borax), too, can be used as crosslinking agents.

As the dichroic dye there may be used any of azo-based dyes, stilbene-based dyes, pyrazolone-based dyes, triphenylmethane-based dyes, oxazine-based dyes, thiazine-based dyes and anthraquinone-based dyes. The dichroic dye is preferably water-soluble. The dichroic dye preferably has a hydrophilic substituent (e.g., sulfo, amino, hydroxyl).

Examples of the dichroic dye employable herein include C. I. direct yellow 12, C. I. direct orange 39, C. I. direct orange 72, C. I. direct red 39, C. I. direct red 79, C. I. direct red 81, C. I. direct red 83, C. I. direct red 89, C. I. direct violet 48, C. I. direct blue 67, C. I. direct blue 90, C. I. direct green 59, and C. I. direct red 37. For the details of the dichroic dye, reference can be made to JP-A-1-161202, JP-A-1-172906, JP-A-1-172907, JP-A-1-183602, JP-A-1-248105, JP-A-1-265205, and JP-A-7-261024. The dichroic dye is used in the form of free acid, alkaline metal salt, ammonium salt or amine salt. By blending two or more dichroic dyes, polarizing films having various hues can be produced. A polarizing film comprising a compound (dye) which assumes black when the polarization axes are disposed perpendicular to each other or a polarizing film or plate which has various dichroic molecules incorporated therein such that black is assumed is excellent in single plate transmission and percent polarization to advantage.

In order to enhance the contrast ratio of the liquid crystal display device, the polarizing plate to be used preferably has a high transmission as well as a high polarization. The transmission of the polarizing plate at a wavelength of 550 nm is preferably from 30% to 50%, more preferably from 35% to 50%, most preferably from 40% to 50% (The maximum single plate transmission of the polarizing plate is 50%). The polarization of the polarizing plate at a wavelength of 550 nm is preferably from 90% to 100%, more preferably from 95% to 100%, most preferably from 99% to 100%.

The polarizing film and the optically anisotropic layer, the polarizing film and the support or the polarizing film and the alignment layer can be disposed with an adhesive interposed therebetween. As the adhesive there may be used a polyvinyl alcohol-based resin (including acetoacetyl group-, sulfone group-, carboxyl group- or oxyalkylene group-modified polyvinyl alcohols) or an aqueous solution of a boron compound. Preferred among these adhesives is polyvinyl alcohol-based resin. The dried thickness of the adhesive layer is preferably from 0.01 μm to 10 μm, particularly preferably from 0.05 μm to 5 μm.

### (Production of Ellipsoidal Polarizing Plate)

In the production of the polarizing film, the binder is preferably stretched at an angle of from 10° to 80° from the longitudinal direction (MD direction) of the polarizing film (stretching method) or rubbed (rubbing method) before being dyed with iodine or dichroic dye from the standpoint of yield. The tilt angle preferably coincides with the angle of the transmission axis of two sheets of polarizing plate stuck to the both sides of the liquid crystal cell constituting LCD with respect to the longitudinal or crosswise direction of the liquid crystal cell.

The tilt angle is normally 45°. In recent years, however, transmission type, reflection type and semi-transmission type liquid crystal display devices the tile angle of which are not necessarily 45° have been developed. It is preferred that the stretching direction be arbitrarily adjusted according to the design of LCD.

In the stretching method, the draw ratio is preferably from 2.5 to 30.0, more preferably from 3.0 to 10.0. Stretching can be effected in a dry process in the air. Alternatively, stretching may be effected in a wet process in water. The draw ratio in the dry stretching process is preferably from 2.5 to 5.0. The draw ratio in the wet stretching process is preferably from 3.0 to 10.0. Stretching may be effected batchwise, including oblique stretching. When stretching is effected batchwise, the film can be stretched more uniformly even at a high draw ratio. The film may be stretched crosswise or longitudinally to some extent (such that the crosswise shrinkage can be prevented) before being obliquely stretched.

Stretching can be carried out by effecting tenter stretching in biaxial stretching in manners which are different from right to left. The aforementioned biaxial stretching method is the same as the stretching method practiced in the ordinary film making method. In the biaxial stretching method, the film is stretched at speeds which are different from right to left. It is therefore necessary that the thickness of the unstretched binder film differ from right to left. In the film making method involving flow casting, the die used can be tapered to make the flow rate of binder solution different from right to left.

Thus, a binder film which has been obliquely stretched at an angle of from 10° to 80° from MD direction of the polarizing film is produced.

As the aforementioned rubbing treatment there may be used one which is widely employed as liquid crystal alignment for LCD. In some detail, the surface of the alignment layer may be rubbed with paper, gauze, felt, rubber, nylon, polyester fiber or the like in a constant direction to attain alignment. In general, the surface of the film is rubbed with a cloth having fibers having uniform length and thickness planted uniformly thereon several times. Rubbing is preferably effected using a rubbing roll having a roundness, a cylindricality and a deflection (eccentricity) of 30 μm or less. The angle of lapping of the film on the rubbing roll is preferably from 0.1° to 90°. However, as disclosed in JP-A-8-160430, stable rubbing can be made by winding the film on the rubbing roll at a lapping angle of 360° or more.

In the case where a film of continuous length is subjected to rubbing, it is preferred that the film be conveyed at a speed of from 1 to 100 m/min while being kept at a constant tension. The rubbing roll is preferably arranged swingable in the horizontal direction against the direction of conveyance of the film so that arbitrary rubbing angle can be predetermined. A proper rubbing angle is predetermined within a range of from 0° to 60°. In the case where the film is used in liquid crystal display devices, the rubbing angle of is preferably from 40° to 50°, particularly preferably 45°.

A polymer film is preferably provided on the surface of the polarizing film on the side thereof opposite the optically anisotropic layer (optically anisotropic layer/polarizing film/polymer film). The polymer film to be used herein is not specifically limited. For example, any of protective films to be used in the related art polarizing plates may be used.

### (Liquid Crystal Display Device)

The invention also concerns a liquid crystal display device comprising an optically-compensatory sheet of the invention or an ellipsoidal polarizing plate of the invention. Preferred embodiments of optically anisotropic layer in various liquid crystal modes of liquid crystal display device will be described hereinafter.

### (TN Mode Liquid Crystal Display Device)

A TN mode liquid crystal cell is most widely used as a color TFT liquid crystal display device. For details, reference can be made to numeral literatures.

Referring to the alignment in the liquid crystal cell during the black display of TN mode, rod-shaped liquid crystal molecules are oriented vertically at the central part of the cell but horizontally in the vicinity of the substrate of the cell.

The rod-shaped liquid crystal compounds in the central portion of the cell can be compensated by homeotropically-aligned (horizontal alignment having disc surface oriented horizontally) discotic liquid crystal compounds or (transparent) support while the rod-shaped liquid crystal compounds in the vicinity of the substrate in the cell can be compensated by hybrid-aligned (alignment involving change of tilt of major axis with distance from polarizing film) discotic liquid crystal compounds.

Further, the rod-shaped liquid crystal compounds in the central portion of the cell can be compensated by homogeneously-aligned (horizontal alignment having major axis oriented horizontally) rod-shaped liquid crystal compounds or (transparent) support while the rod-shaped liquid crystal compounds in the vicinity of the substrate in the cell can be compensated by hybrid-aligned disc-shaped liquid crystal compounds.

The homeotropically-aligned liquid crystal compounds are aligned in such an arrangement that the angle of the average alignment direction of major axis of the liquid crystal compounds with respect to the surface of the polarizing film is from 85° to 95°.

The homogeneously-aligned liquid crystal compounds are aligned in such an arrangement that the angle of the average alignment direction of major axis of the liquid crystal compounds with respect to the surface of the polarizing film is less than 5°.

The hybrid-aligned discotic liquid crystal compounds are aligned in such an arrangement that the angle of the average alignment direction of major axis of the liquid crystal compounds with respect to the surface of the polarizing film is preferably more than 15°, more preferably from 15° to 85°.

The hybrid-aligned rod-shaped liquid crystal compounds are aligned in such an arrangement that the angle of the average alignment direction of major axis of the liquid crystal compounds with respect to the surface of the polarizing film is preferably 75° or less, more preferably from 5° to 75°.

In the case where the liquid crystal display device according to the present embodiment comprises an optically-compensatory sheet of the invention, the Rth retardation value and Re retardation value of the optically anisotropic layer are preferably from 40 nm to 200 nm and from 0 nm to 70 nm, respectively.

The tilt angle of the discotic liquid crystal compound at the air interface can be measured by the aforementioned measuring method.

### (OCB Mode Liquid Crystal Display Device)

An OCB mode liquid crystal cell is a liquid crystal cell of bend alignment mode wherein rod-shaped liquid crystal molecules are oriented in substantially opposing directions (symmetrically) from the upper part to the lower part of the liquid crystal cell. A liquid crystal display device comprising a bend alignment mode liquid crystal cell comprises devices disclosed in U.S. Pat. Nos. 4,583,825 and 5,410,422 oriented symmetrically with each other from the upper part to the lower part of the liquid crystal cell. Therefore, the bend alignment mode liquid crystal cell has a self optical compensation capacity. Accordingly, this liquid crystal mode is also called OCB (Optically Compensatory Bend) liquid crystal mode. In OCB mode liquid crystal cell, too, rod-shaped liquid crystal molecules are oriented vertically at the central part of the liquid crystal cell but are oriented horizontally in the vicinity of the substrate of the cell during black display as in TN mode. Since the orientation of the liquid crystal layer in TN mode during black display is the same state, the preferred range of the optical properties is the same as those in TN mode. However, OCB mode has its liquid crystal compounds aligned erected in a wider area at the center of the cell than TN mode. Therefore, the optically anisotropic layer having discotic liquid crystal compounds homeotropically aligned or rod-shaped liquid crystal compounds homogenously aligned needs to be somewhat adjusted in retardation value. In some detail, when an optically-compensatory sheet having an optically anisotropic layer having discotic liquid crystal compounds homeotropically aligned or an optically anisotropic layer having rod-shaped liquid crystal compounds homogeneously aligned is used, the resulting Rth retardation value and Re retardation value are preferably from 150 nm to 500 nm and from 20 nm to 70 nm, respectively.

### (VA Mode Liquid Crystal Display Device)

In a VA mode liquid crystal cell, rod-shaped liquid crystal molecules are vertically oriented when no voltage is applied.

VA mode liquid crystal cells include (1) liquid crystal cell in VA mode in a narrow sense in which rod-shaped liquid crystal molecules are oriented substantially vertically when no voltage is applied but substantially horizontally when a voltage is applied (as disclosed in JP-A-2-176625). In addition to the VA mode liquid crystal cell (1), there have been provided (2) liquid crystal cell of VA mode which is multidomained to expand the viewing angle (MVA mode) (as disclosed in SID97, Digest of Tech. Papers (preprint) 28 (1997), 845), (3) liquid crystal cell of mode in which rod-shaped molecules are oriented substantially vertically when no voltage is applied but oriented in twisted multidomained mode when a voltage is applied (n-ASM mode, CPA mode) (as disclosed in Preprints of Symposium on Japanese Liquid Crystal Society Nos. 58 to 59, 1998 and (4) liquid crystal cell of SURVAIVAL mode (as reported in LCD International 98).

During black display of VA mode liquid crystal display device, most of the rod-shaped liquid crystal molecules in the liquid crystal cell are oriented vertically. It is therefore preferred that the liquid compound be compensated by an optically anisotropic layer having a discotic compound homeotropically aligned therein or an optically anisotropic layer having rod-shaped liquid crystal molecules homogeneously aligned therein while the viewing angle dependence of the polarizing plate be compensated by an optically anisotropic layer having rod-shaped liquid crystal molecules homogeneously aligned therein wherein the angle of the average alignment direction of the major axis of the rod-shaped liquid crystal molecules with respect to the direction of the transmission axis of the polarizing film is less than 5°. The optically-compensatory sheet or ellipsoidal polarizing plate of the invention may be used for any of the aforementioned optical compensation systems.

### (Other Liquid Crystal Display Devices)

ECB mode and STN mode liquid crystal display devices can be optically compensated in the same idea as mentioned above.

### EXAMPLE

The invention will be further described in the following examples. The materials, reagents, proportions, procedures, etc. described in the following examples may be properly changed without departing from the spirit of the invention. Therefore, the scope of the invention is not limited to the following examples.

### 1. Synthesis of Fluoroaliphatic Group-Containing Polymer

### 1-1. Synthesis of Fluoroaliphatic Group-Containing Polymer (P-33)

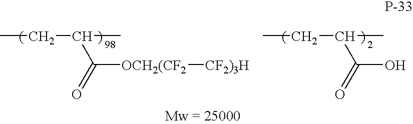

Into a reaction vessel equipped with an agitator and a reflux condenser were charged 39.13 g of 1H, 1H, 7H-dodecafluoroheptyl acrylate, 0.80 g of acrylic acid, 1.1 g of 2,2′-azobisisobutyrate and 30 g of 2-butanone. The reaction mixture was then heated to 78° C. in a nitrogen atmosphere for 6 hours until the reaction was completed. The resulting product had a weight-average molecular weight of 2.5×10⁴.

### 1-2. Synthesis of Fluoroaliphatic Group-Containing Polymers (P-34), (P-81), (P-136) and (P-138)

Fluoroaliphatic group-containing polymers (P-34), (P-81), (P-136) and (P-138) were prepared in the same manner as in the synthesis of the fluoroaliphatic group-containing polymer (P-33).

### Example A1

### 2. Preparation of Optically-Compensatory Sheet

### 2-1. Preparation of Transparent Support

### 2-1-1. Preparation of Inner Layer Dope (D-1) and Outer Layer Dope (D-2)

Into a mixing tank were charged the following components which were then heated to 30° C. with stirring to make a solution of these components. Thus, a cellulose acetate solution was prepared

### <Formulation of Inner Layer Dope (D-1) and Outer Layer Dope (D-2) (Parts by Mass)>

| | D-1 | D-2 |
|---|---|---|
| Cellulose acetate having a percent acetylation of 60.9% | 100 | 100 |
| Triphenyl phosphate (plasticizer) | 7.8 | 7.8 |
| Biphenyl diphenyl phosphate (plasticizer) | 3.9 | .9 |
| Methylene chloride (first solvent) | 293 | 314 |
| Methanol (second solvent) | 71 | 76 |
| 1-Butanol (third solvent) | 1.5 | 1.6 |
| Particulate silica (AEROSIL R972, produced by NIPPON | 0 | 0.8 |
| AEROSIL CO., LTD.) | | |
| Retardation raising agent shown below | 1.7 | 0 |

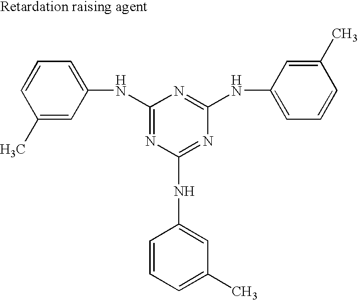

The inner layer dope and the outer layer dope thus obtained were each then flow-casted over a drum which had been chilled to 0° C. using a three-layer co-casting die. When the residual solvent content reached 70% by mass, the film was then peeled off the drum. While being conveyed at a draw ratio of 110% in the conveying direction with its edges fixed by a pin tenter, the film thus peeled was then dried at 80° C. When the residual solvent content reached 10%, the film was then dried at 110° C. Thereafter, the film was dried at 140° C. for 30 minutes to prepare a cellulose acetate film (outer layer: 3 μm; inner layer: 74 μm; outer layer: 3 μm) having a residual solvent content of 0.3% by mass. The transparent support, which is the cellulose acetate film thus prepared, was then measured for optical properties.

The transparent support thus obtained had a width of 1,340 mm and a thickness of 80 μm. The transparent support was then measured for retardation value (Re) at a wavelength of 500 nm using a Type KOBRA21ADH automatic birefringence meter (produced by Ouji Scientific Instruments Co., Ltd.). The result was 6 nm. The transparent support was also measured for retardation value (Rth) at a wavelength of 500 nm. The result was 83 nm.

The transparent support thus obtained was dipped in a 2.0 N solution of potassium hydroxide (25° C.) for 2 minutes, neutralized with sulfuric acid, washed with purified water, and then dried. The transparent support thus treated was then measured for surface energy by contact angle method. The result was 63 mN/m.

The various steps will be further described hereinafter.

### 2-2. Formation of Alignment Film

An alignment film coating solution having the following formulation was then spread over the surface of the transparent support, dried with 60° C. hot air for 60 seconds and then with 90° C. hot air for 150 seconds to form an alignment film resin layer.

### <Formulation of Alignment Film Coating Solution>

| | |
|---|---|
| Modified polyvinyl alcohol having the following | 10 parts by mass |
| general formula | |
| Water | 371 parts by mass |
| Methanol | 119 parts by mass |
| Glutaraldehyde (crosslinking agent) | 0.5 parts by mass |

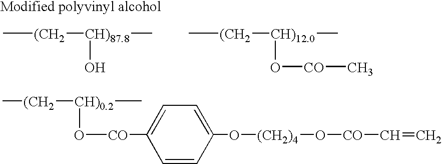

### 2-3. Formation of Optically Anisotropic Layer

Subsequently, the following coating solution was spread over the surface of the substrate in an amount of 5.5 cc/m² using a #3.2 wire bar. The coated material was then heated in a 130° C. constant temperature zone for 2 minutes to cause discotic liquid crystal compound molecules to be aligned. Subsequently, the coated material was irradiated with ultraviolet rays from a 120 W/cm high pressure mercury vapor lamp in a 35° C. atmosphere for 1 minute to cause the discotic liquid crystal compound to be polymerized. Thereafter, the material was allowed to cool to room temperature to form an optically anisotropic layer Thus, an optically-compensatory sheet was prepared.

### <Formulation of Coating Solution for Optically Anisotropic Layer>

The following components were dissolved in 102 kg of methyl ethyl ketone to prepare a coating solution.

| | |
|---|---|
| Discotic liquid crystal compound (1) shown below | 41.01 parts by mass |
| Ethylene oxide-modified trimethylol propane | 3.61 parts by mass |
| triacrylate (V#360, produced by OSAKA | |
| ORGANIC CHEMICAL INDUSTRY LTD.) | |
| Cellulose acetate butyrate (CAB551-0.2, produced | 0.045 parts by mass |
| by Eastman Chemical Inc.) | |
| Exemplified compound of fluoroaliphatic group- | 0.23 parts by mass |
| containing polymer (P-136) | |
| Exemplified compound of fluoroaliphatic group- | 0.18 parts by mass |
| containing polymer (P-33) | |
| Photopolymerization initiator (Irgacure 907, | 1.35 parts by mass |
| produced by Ciba Geigy Inc.) | |
| Sensitizer (Kayacure DETX, produced by Nippon | 0.45 parts by mass |
| Kayaku Corporation) | |

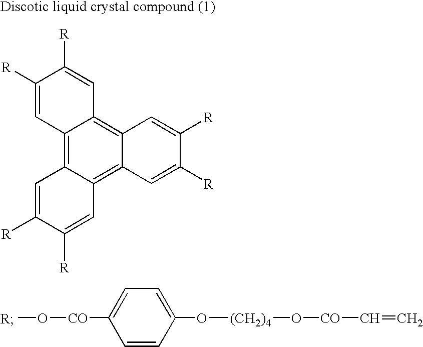

The Re retardation value of the optically anisotropic layer measured at a wavelength of 546 nm using a Type KOBRA-21ADH automatic birefringence meter (produced by Ouji Scientific Instruments Co., Ltd.) was 50 nm. The optically compensatory sheet thus obtained was also observed for unevenness with the polarizing plate arranged in crossed nicols. As a result, no unevenness was detected even as viewed at front ways and at an angle of 60° from the line normal to the surface of the film.

### <<Evaluation of Tilt Angle>>

For the evaluation of the angle of tilt of molecules in the vicinity of alignment layer and in the vicinity of air interface in the optically anisotropic layer, a Type APE-100 ellipsometer (produced by Shimadzu Corporation) was used. With the angle of observation varied, the retardation of the optically anisotropic layer was measured. The measurements were then subjected to calculation according to the method described in “Jpn. J. Appl. Phys.”, vol, 36 (1997). pp. 143-147. The measuring wavelength was 632.8 nm. The results are set forth in Table 1.

### <<Evaluation of Alignment Order Parameter S>>

The alignment order parameter S of the molecules in the optically anisotropic layer in the vicinity of air interface (S₁), in the vicinity of air interface on the side of the optically anisotropic layer opposite the former air interface (alignment film interface) (S₂), and in the interior of the optically anisotropic layer (S₃) were measured by the aforementioned method. The results are set forth in Table 1.

### <<Evaluation of Haze>>

The film thus obtained was measured for haze using a Model 100DP haze meter (produced by NPPON DENSHOKU KOGYO CO., LTD.).

### 3. Preparation of Polarizing Plate

The optically compensatory sheet was stuck to one side of the polarizer with a polyvinyl alcohol-based adhesive. A triacetyl cellulose film having a thickness of 80 μm (TD-80U, produced by Fuji Photo Film Co., Ltd.) was saponified, and then stuck to the other side of the polarizer with a polyvinyl alcohol-based adhesive.

The polarizing plate was arranged such that the transmission axis of the polarizer and the slow axis of the polymer substrate, which is the support of the optically-compensatory sheet, were disposed parallel to each other. The transmission axis of the polarizer and the slow axis of the aforementioned triacetyl cellulose film were disposed perpendicular to each other. Thus, a polarizing plate was prepared.

### <Evaluation of Viewing Angel>>

A pair of polarizing plates were peeled off a Type AQUOS LC20CIS liquid crystal display device comprising a TN type liquid crystal cell (produced by SHARP CORPORATION). Instead of these polarizing plates, the polarizing plate (HB-1) prepared above was stuck to the observer side and the backlight side of the liquid crystal cell with an adhesive in such an arrangement that the optically-compensatory sheet was on the liquid crystal side. The transmission axis of the polarizing plate on the observer side and the transmission axis of the polarizing plate on the backlight side were disposed such that O mode was established.

The liquid crystal display device thus prepared was then measured for viewing angle from black display (L1) to white display (L8) using a Type EZ-Contrast 160D (produced by ELDIM). The range within which the contrast ratio (white transmission/black transmission) is 10 or more in the vertical direction and horizontal direction and there is no gradation inversion (inversion of L1 with L2) on black side was then determined. The measurements are set forth in Table 1.

### <<Evaluation of Unevenness>>

The display panel of the liquid crystal display devices was entirely adjusted half-tone to make evaluation of unevenness.

### Examples A2 to A4; Comparative Examples A1 to A3

Optically-compensatory sheets were prepared in the same manner as in Example A1 except that the UV polymerization temperature, the kind and added amount of fluoroaliphatic group-containing polymer and the kind and added amount of CAB compound were changed as shown in Table 1. These optically-compensatory sheets were then evaluated for alignment order parameter, tilt angle and unevenness in the same manner as described above, The results are set forth in Table 1.

### Examples B1 to B4; Comparative Examples B1 to B5

Optically-compensatory sheets were prepared in the same manner as in Example A1 except that the UV polymerization temperature, the kind and added amount of fluoroaliphatic group-containing polymer and the kind and added amount of CAB compound were changed as shown in Table 2. In Comparative Example B5, as an optically-compensatory sheet there was used a Type WV-SA (07D) optically-compensatory sheet (produced by Fuji Photo Film Co., Ltd.). These optically-compensatory sheets were then evaluated for alignment order parameter, tilt angle and unevenness in the same manner as described above. The results are set forth in Table 2.

TABLE 1

| | Fluoro-aliphatic | | Optically-compensatory sheet | | | | | | | | | | | |
|---|---|---|---|---|---|---|---|---|---|---|---|---|---|---|
| | group-containing | | Optically anisotropic layer | | | | | | | | | | | |
| | polymer | | CAB compound | | UV | | Alignment order | | | | | Polarizing plate | | |
| | | Parts by | | Parts by | polymerization | Thickness | parameter S | | | Tilt angle [°] | | Viewing angle [°] | | |
| | Kind | mass | Kind^{*1} | mass | temp. (° C.) | (μm) | S₁ | S₂ | S₃ | θ2 | θ1 | Vertical | Horizontal | Uneveness |
| Example A1 | P-33 | 0.18 | 1 | 0.045 | 35 | 1.25 | 0.65 | 0.65 | 0.65 | 10 | 85 | 140 | 155 | Good |
| | P-136 | 0.23 | | | | | | | | | | | | |
| Example A2 | P-34 | 0.12 | 1 | 0.045 | 55 | 1.35 | 0.60 | 0.60 | 0.60 | 10 | 85 | 137 | 153 | Good |
| | P-136 | 0.23 | | | | | | | | | | | | |
| Example A3 | P-33 | 0.20 | 1 | 0.09 | 35 | 1.31 | 0.40 | 0.55 | 0.65 | 11 | 85 | 138 | 152 | Good |
| | P-136 | 0.23 | | | | | | | | | | | | |
| Example A4 | P-34 | 0.15 | 2 | 0.045 | 60 | 1.37 | 0.40 | 0.50 | 0.55 | 11 | 85 | 133 | 150 | Good |
| | P-136 | 0.23 | | | | | | | | | | | | |
| Comparative | P-34 | 0.18 | 1 | 0.9 | 90 | 1.60 | 0.20 | 0.25 | 0.30 | 11 | 85 | 121 | 135 | Good |
| Example A1 | P-136 | 0.23 | | | | | | | | | | | | |
| Comparative | P-34 | 0.18 | 1 | 1.35 | 35 | 1.42 | 0.25 | 0.25 | 0.35 | 12 | 85 | 120 | 134 | Good |
| Example A2 | P-136 | 0.23 | | | | | | | | | | | | |
| Comparative | P-33 | 0.36 | — | — | 90 | 1.50 | 0.30 | 0.30 | 0.30 | 11 | 85 | 124 | 143 | Not good |
| Example A3 | P-136 | 0.23 | | | | | | | | | | | | |

^{*1}CAB compound 1: CAB551-0.2, produced by Eastman Chemical Co., Ltd.
CAB compound 2: CAB531-1, Eastman Chemical Co., Ltd.

TABLE 2

| | Fluoro-aliphatic | | Optically-compensatory sheet | | | | | | | | | | |
|---|---|---|---|---|---|---|---|---|---|---|---|---|---|
| | group-containing | | Optically anisotropic layer | | | | | | | | | | |
| | polymer | | CAB compound | | UV | | Alignment order | Tilt | | | Polarizing plate | | |
| | | Parts by | | Parts by | polymerization | Thickness | parameter S | angle [°] | | Haze | Viewing angle [°] | | Un- |
| | Kind | mass | Kind^{*1} | mass | temp. (° C.) | (μm) | S₁ | θ2 | θ1 | (%) | Vertical | Horizontal | eveness |
| Example B1 | P-33 | 0.01 | 1 | 0.34 | 60 | 1.28 | 0.45 | 10 | 65 | 0.33 | 134 | 152 | Good |
| | P-136 | 0.18 | 2 | 0.11 | | | | | | | | | |
| Example B2 | P-34 | 0.01 | 1 | 0.34 | 60 | 1.32 | 0.47 | 10 | 66 | 0.47 | 135 | 151 | Good |
| | P-138 | 0.18 | 2 | 0.11 | | | | | | | | | |
| Example B3 | P-33 | 0.01 | 1 | 0.34 | 60 | 1.31 | 0.44 | 11 | 66 | 0.44 | 134 | 151 | Good |
| | P-136 | 0.27 | 2 | 0.11 | | | | | | | | | |
| Example B4 | P-33 | 0.01 | 1 | 0.34 | 60 | 1.29 | 0.49 | 11 | 66 | 0.49 | 134 | 152 | Good |
| | P-136 | 0.36 | 2 | 0.11 | | | | | | | | | |
| Comparative | P-136 | 0.18 | 1 | 0.34 | 60 | 1.5 | 0.41 | 11 | 50 | 0.41 | 120 | 141 | Good |
| Example B1 | — | — | 2 | 0.11 | | | | | | | | | |
| Comparative | P81 | 0.18 | 1 | 0.34 | 60 | 1.55 | 0.42 | 11 | 51 | 0.42 | 121 | 135 | Not |
| Example B2 | — | — | 2 | 0.11 | | | | | | | | | good |
| Comparative | — | — | 1 | 0.34 | 60 | 1.52 | 0.40 | 12 | 50 | 0.40 | 115 | 134 | Bad |
| Example B3 | | | 2 | 0.11 | | | | | | | | | |
| Comparative | — | — | 1 | 0.9 | 60 | 1.57 | 0.3 | 11 | 63 | 0.3 | 124 | 143 | Good |
| Example B4 | | | 2 | 0.22 | | | | | | | | | |

| | | | | | | | | | | |
|---|---|---|---|---|---|---|---|---|---|---|
| Comparative | WV-SA^{*2} (related art product) | 60 | 1.59 | 0.29 | 11 | 66 | 0.29 | 134 | 151 | Good |
| Example B5 | | | | | | | | | | |

^{*1}CAB compound 1: CAB551-0.2, produced by Eastman Chemical Co., Ltd.
CAB compound 2: CAB531-1, Eastman Chemical Co., Ltd.
^{*2}WV-SA: Optically-compensatory sheet “WV-SA (07D)”, produced by Fuji Photo Film Co., Ltd.

As can be seen in the results of Table 1 above, the optically-compensatory sheets of Examples A1 to A4 exhibit the same or better optical compensation effect as the optically-compensatory sheets of Comparative Examples A1 to A3, demonstrating that these optically-compensatory sheets contribute to the enhancement of the viewing angle contrast of liquid crystal display device. The optically anisotropic layers contained in the optically-compensatory sheets of Examples A1 to A4 satisfy the requirement that the maximum value Sₘₐₓ of alignment order parameter S of molecules be not smaller than 0.4. As a result, it can be appreciated that although the inventive optically-compensatory sheets are thinner than the optically anisotropic layers contained in the optically-compensatory sheets of Comparative Examples A1 to A3, they can exhibits the same or better optical compensation effect than the comparative products.

As can be seen in the results of Table 2 above, the optically-compensatory sheets comprising an optically anisotropic layer having a surface alignment order parameter S₁ of from not smaller than 0.35 to not greater than 0.7 wherein the tilt angle of liquid crystal compound at the air interface is from 55° to 75° can be reduced in the thickness of the optically anisotropic layer despite the same optical compensation effect as the related art optical compensation sheet {“WV-SA (07D)”, produced by Fuji Photo Film Co., Ltd.}).

### INDUSTRIAL APPLICABILITY

The invention provides an optically-compensatory sheet which satisfies the requirement that the liquid crystal compound in the optically anisotropic layer has Sₘₐₓ of 0.4 or more, or S₁ of from 0.35 to 0.7 to exhibit good optical compensation effect even when the optically anisotropic layer has a small thickness. Further, the optically-compensatory sheet of the invention can be produced at a high productivity and exhibits a good haze. In other words, the invention can provide an optically-compensatory sheet which exhibits a good optical compensation effect, contributes to the enhancement of viewing angle when applied to image display devices and can be produced at a high productivity. The invention also can provide an ellipsoidal polarizing plate which contributes to the enhancement of the viewing angle of liquid crystal display devices and a liquid crystal display device having excellent viewing angle properties and a high display fidelity.

The entire disclosure of each and every foreign patent application from which the benefit of foreign priority has been claimed in the present application is incorporated herein by reference, as if fully set forth.

1. An optically-compensatory sheet comprising:
an optically anisotropic layer that comprises a liquid crystal compound,
wherein the liquid crystal compound in the optically anisotropic layer has Sₘₐₓ of 0.4 or more,
in which Sₘₐₓ is a maximum value among S₁, S₂ and S₃;
S1 is an alignment order parameter in a vicinity of an interface between the optically anisotropic layer and air;
S2 is an alignment order parameter in a vicinity of an interface on a side opposite to the interface between the optically anisotropic layer and air; and
S₃ is an alignment order parameter in an interior of the optically anisotropic layer.

2. The optically-compensatory sheet according to claim 1,
wherein S₃ is Sₘₐₓ.

3. The optically-compensatory sheet according to claim 1,
wherein S₁ is substantially same as Sₘₐₓ.

4. The optically-compensatory sheet according to claim 1,
wherein S₂ is substantially same as Sₘₐₓ.

5. The optically-compensatory sheet according to claim 1,
wherein S₁, S₂ and S₃ are substantially same.

6. The optically-compensatory sheet according to claim 1,
wherein θ1 is greater than θ2,
in which θ1 is an angle of inclination of the liquid crystal compound in the vicinity of the interface between the optically anisotropic layer and air; and
θ2 is an angle of inclination of the liquid crystal compound in the vicinity of the interface on the side opposite to the interface between the optically anisotropic layer and air.

7. The optically-compensatory sheet according to claim 6,
wherein θ1 is 40 degrees or more.

8. The optically-compensatory sheet according to claim 1,
wherein Sₘₐₓ is 0.5 or more.

9. An optically-compensatory sheet comprising:
an optically anisotropic layer that comprises a liquid crystal compound,
wherein the liquid crystal compound in the optically anisotropic layer has θ1 of 55 degrees or more and S₁ of from 0.35 to 0.7,
in which θ1 is an angle of inclination of the liquid crystal compound in a vicinity of an interface between the optically anisotropic layer and air; and
S₁ is an alignment order parameter of the liquid crystal compound in the vicinity of the interface between the optically anisotropic layer and air.

10. The optically-compensatory sheet according to claim 1,
wherein the optically anisotropic layer further comprises at least two fluoroaliphatic group-containing polymers, each of which comprises a repeating unit derived from a monomer represented by formula (1): 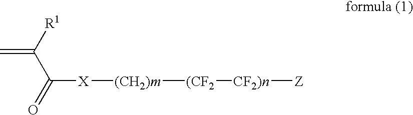
wherein R¹ represents a hydrogen atom or methyl group;
X represents an oxygen atom, sulfur atom or —N(R²)— in which R² represents a hydrogen atom or C₁-C₄ alkyl group;
Z represents a hydrogen atom or fluorine atom;
m represents an integer of from 1 to 6; and
n represents an integer of from 2 to 4.

11. The optically-compensatory sheet according to claim 10,
wherein Z represents a hydrogen atom.

12. The optically-compensatory sheet according to claim 10,
wherein at least one fluoroaliphatic group-containing polymer of the at least two fluoroaliphatic group-containing polymers further comprises a repeating unit derived from a monomer represented by formula (2): 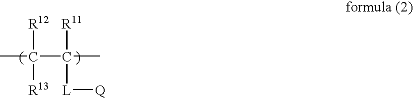
wherein R¹¹, R¹² and R¹³ each independently represents a hydrogen atom or a substituent;
L represents a divalent connecting group which is a combination of one or more connecting groups selected from a group consisting of single bond; —O—; —CO—; —NR⁴— in which R⁴ represents a hydrogen atom, alkyl group, aryl group or aralkyl group; —S—; —SO₂—; —P(═O)(OR⁵)— in which R⁵ represents an alkyl group, aryl group or aralkyl group; alkylene group and arylene group; and
Q represents a carboxyl group (—COOH), salt thereof, sulfo group (—SO₃H), salt thereof, phosphonoxy {—OP(═O)(OH)₂}, salt thereof or alkylene group containing poly(alkyleneoxy) group.

13. The optically-compensatory sheet according to claim 1,
wherein the liquid crystal compound is a discotic compound.

14. An ellipsoidal polarizing plate comprising:
a polarizing film; and
an optically-compensatory sheet according to claim 1.

15. A liquid crystal display device comprising an optically-compensatory sheet according to claim 1.

16. The optically-compensatory sheet according to claim 9, wherein the optically anisotropic layer further comprises at least two fluoroaliphatic group-containing polymers, each of which comprises a repeating unit derived from a monomer represented by formula (1): 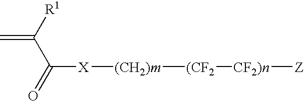
wherein R¹ represents a hydrogen atom or methyl group;
X represents an oxygen atom, sulfur atom or —N(R²)— in which R² represents a hydrogen atom or C₁-C₄ alkyl group;
Z represents a hydrogen atom or fluorine atom;
m represents an integer of from 1 to 6; and
n represents an integer of from 2 to 4.

17. The optically-compensatory sheet according to claim 9, wherein the liquid crystal compound is a discotic compound.

18. An ellipsoidal polarizing plate comprising:
a polarizing film; and
an optically-compensatory sheet according to claim 9.

19. A liquid crystal display device comprising an optically-compensatory sheet according to claim 9.
